(19) United States
(12) Patent Application Publication
(10) Pub. No.: US 2023/0084305 A1
(43) Pub. Date: Mar. 16, 2023

(54) MAGNETIC DRUG DELIVERY CAPSULES AND SYSTEMS

(71) Applicant: PILLUETTE LTD., Ramat Hasharon (IL)

(72) Inventors: David KELRICH, Ramat Hasharon (IL); Itzhak POMERANTZ, Kefar Sava (IL)

(21) Appl. No.: 17/794,968

(22) PCT Filed: Jan. 21, 2021
(86) PCT No.: PCT/IL2021/050065

§ 371 Date: Jul. 24, 2022

Related U.S. Application Data
(60) Provisional application No. 62/966,036, filed on Jan. 27, 2020.

(51) Int. Cl. A61M 31/00; A61K 9/00; A61K 9/48

(52) U.S. Cl. CPC A61M 31/002; A61K 9/0053; A61K 9/48; A61K 9/0009

(57) ABSTRACT

Provided herein are magnetically controlled drug delivery capsules configured for oral administration and systems adapted for drug release at a desired location of the GI tract and methods of using same for treating a disease or a disorder.

### FIELD OF THE INVENTION

Provided herein are magnetically controlled drug delivery capsules and systems adapted for drug release at a desired location of the GI tract and methods of using same for treating a disease or a disorder.

### BACKGROUND OF THE INVENTION

The therapeutic benefit of many orally administered pharmaceuticals can be improved by enhancing their bioavailability. This can be achieved by increased residence time in the GI tract, in particular, in specific regions within the GI tract. The use of magnetic pills or capsules containing pharmaceuticals has been suggested for prolonging the residence time of pharmaceuticals in the GI tract. For example, International Publication No. WO 2018/073726 by the inventors of the present application, the entire contents of which incorporated herein by reference, discloses a magnetically controlled capsule that can stay at desired GI locations for an extended period of time.

U.S. Pat. No. 8,776,802, the entire contents of which incorporated herein by reference, discloses a method for prolonging localization of therapeutics within the rat gastrointestinal tract, the method comprising guiding a magnetic pill trough the GI tract to an anatomic location of a direct tissue or organ, utilizing an external magnet.

US Patent Application No. 2013/0303847, the entire contents of which incorporated herein by reference, discloses a swallowable device with a soft, compliant exterior, whose shape can be changed through the use of magnetic fields, and which can be locomoted in a rolling motion through magnetic control from the exterior of the user.

U.S. Pat. No. 3,659,600, the entire contents of which incorporated herein by reference, discloses an implantable capsule for delivering drugs when subjected to a magnetic force outside the body and controlled by the user. The capsule comprises an open hollow magnet being movable under a magnetic force, enabling the contact of a medicament in the capsule with a drug-permeable wall. When contacted, the medicament diffuses through the wall into the body.

There remains an unmet need for simple, cost-efficient, accurate, and reliable drug delivery capsules, delivery systems, and methods for using the same for controllable drug release at desired locations in the GI tract.

### SUMMARY OF THE INVENTION

The present invention provides drug delivery capsules, and systems adapted for drug release at a desired location of the GI tract, and methods of using same, for treating various diseases and/or disorders.

Certain embodiments of the present invention may include some, all, or none of the above advantages. Further advantages may be readily apparent to those skilled in the art from the figures, descriptions, and claims included herein. Aspects and embodiments of the invention are further described in the specification herein below and in the appended claims.

According to a first aspect, there is provided a capsule configured for oral administration and drug release at a desired location in gastrointestinal (GI) tract, comprising: at least one magnet configured to output a magnetic field external to the capsule; at least one buffer; and at least one drug chamber comprising at least one opening located at a surface of the at least one drug chamber and at least one pharmaceutical composition, wherein responsive to an erosion of the at least one buffer, an intensity of the external output magnetic field is reduced.

According to some embodiments, the at least one buffer comprises the at least one pharmaceutical composition, and wherein responsive to an erosion of the at least one pharmaceutical composition, the at least one pharmaceutical composition is gradually released through the at least one opening towards the desired location within the interior of the GI tract. According to further embodiments, the capsule further comprises at least one magnetic shunt, whereby prior to the erosion of the pharmaceutical composition, the pharmaceutical composition is positioned between the at least one magnet and the at least one magnetic shunt such that the at least one magnet does not contact the at least one magnetic shunt. According to still further embodiments, responsive to the erosion of the pharmaceutical composition, relative movement is initiated between the at least one magnetic shunt and the at least one magnet such that the at least one magnet approaches the at least one magnetic shunt.

According to further embodiments, the at least one magnet comprises a pair of horse-shoe shaped magnets, the at least one pharmaceutical composition separating the pair of horse-shoe shaped magnets.

According to some embodiments, the at least one buffer comprises at least one biodegradable membrane.

According to some embodiments, the at least one biodegradable membrane separates the at least one magnet from an environment surrounding the capsule. According to further embodiments, responsive to an erosion of the at least one biodegradable membrane, the at least one magnet is exposed to the environment surrounding the capsule.

According to some embodiments, the at least one magnet is configured to erode responsive to contact with fluid of the GI tract.

According to some embodiments, the capsule comprises a corrosive powder, the at least one biodegradable membrane separating the corrosive powder from an environment surrounding the capsule. According to further embodiments, responsive to an erosion of the at least one biodegradable membrane, the corrosive powder is exposed to the environment surrounding the capsule. According to some embodiments, the at least one magnet is configured to erode responsive to contact with a solution of the corrosive powder and fluid of the GI tract.

According to some embodiments, the capsule further comprises at least one partitioning element positioned so as to partition the drug chamber into two fluidly connected first and second compartments; and at least one mobile member configured to be magnetized, the at least one mobile member residing within the second compartment. According to further embodiments, the at least one opening comprises a first opening facing the first compartment and a second opening facing the second compartment, and wherein, responsive to changes in direction of an externally applied magnetic field, the at least one mobile member is configured to alternately move towards and away from the second opening. According to still further embodiments, a predetermined volume of the pharmaceutical composition is released through the first opening responsive to the mobile member moving towards the second opening.

According to some embodiments, the capsule further comprises at least one magnetic shield adapted to magnetically shield the at least one mobile member from the at least one magnet.

According to some embodiments, the at least one partitioning element comprises a first partitioning element and a second partitioning element, the second compartment is formed between the first partitioning element and the second partitioning element, and a third compartment is formed between the second partitioning element and an edge of the capsule, wherein the third compartment is in fluid connection with the second compartment, wherein the at least one opening comprises a first opening facing the first compartment and a second opening facing the third compartment, and wherein, responsive to changes in direction of an externally applied magnetic field, the at least one mobile member is configured to alternately move through the second compartment towards the fluid connection to the third compartment and away from the fluid connection to the third compartment.

According to some embodiments, the capsule further comprises a casing encompassing at least the drug chamber, wherein a predetermined space is defined between the casing and the drug chamber. According to further embodiments, the capsule further comprises at least one spacing element positioned between the drug chamber and the casing, the predetermined space defined by the at least one spacing element.

According to some embodiments, the casing is elastic. In further embodiments, the casing is configured to be compressed against the drug chamber, thereby sealing the at least one opening.

According to some embodiments, the casing comprises a perforated or porous material, the material configured to allow the pharmaceutical composition to be released from the capsule via the at least one opening and the casing.

According to some embodiments, the casing comprises at least one casing opening adapted to enable the insertion of the capsule into the casing for the utilization thereof. In further embodiments, the casing further comprises at least one elongated member configured to seal at least a portion of said at least one casing opening after the insertion thereof.

According to some embodiments, the capsule further comprises at least one opening cover; and at least one metallic element attached to the at least one opening cover, wherein the at least one opening cover is configured to seal the at least one opening responsive to the at least one metallic element being magnetized. According to further embodiments, the capsule further comprises at least one hinge, the at least one opening cover connected to the at least one opening via the at least one hinge, wherein the at least one hinge is configured to allow the at least one opening cover to open into the at least one drug chamber.

According to some embodiments, the capsule further comprises a barrier circumferentially surrounding the at least one opening.

In some embodiments, the geometry of the capsule is selected from a barrel-like shape, sphere, ellipsoid, a cylinder, and combinations thereof.

According to another aspect, there is provided a capsule configured for oral administration and drug release at a desired location in the gastrointestinal (GI) tract, comprising: at least one magnet configured to output a magnetic field external to the capsule; at least one drug chamber exhibiting at least one opening located at a surface of the at least one drug chamber; at least one pharmaceutical composition positioned within the at least one drug chamber; at least one partitioning element positioned so as to partition the drug chamber into two fluidly connected first and second compartments; and at least one mobile member configured to be magnetized, the at least one mobile member residing within the second compartment.

According to some embodiments, the at least one opening comprises a first opening facing the first compartment and a second opening facing the second compartment, and wherein, responsive to changes in direction of an externally applied magnetic field, the at least one mobile member is configured to alternately move towards and away from the second opening.

According to some embodiments, a predetermined volume of the pharmaceutical composition is released through the first opening responsive to the mobile member moving towards the second opening.

According to some embodiments, the capsule further comprises at least one magnetic shield adapted to magnetically shield the at least one mobile member from the at least one magnet.

According to some embodiments, the pharmaceutical composition is encompassed within the first compartment.

According to some embodiments, the capsule comprises a plurality of magnets, each configured to output a magnetic field external to the capsule. According to further embodiments, the capsule comprises a plurality of magnetic shield corresponding to the plurality of the magnets.

According to some embodiments, the at least one partitioning element comprises a first partitioning element and a second partitioning element, the second compartment is formed between the first partitioning element and the second partitioning element, and a third compartment is formed between the second partitioning element and an edge of the capsule, wherein the third compartment is in fluid connection with the second compartment, wherein the at least one opening comprises a first opening facing the first compartment and a second opening facing the third compartment, and wherein, responsive to changes in direction of an externally applied magnetic field, the at least one mobile member is configured to alternately move through the second compartment towards the fluid connection to the third compartment and away from the fluid connection to the third compartment.

According to some embodiments, the capsule further comprises a casing encompassing at least the drug chamber, wherein a predetermined space is defined between the casing and the drug chamber. According to further embodiments, the capsule further comprises at least one spacing element positioned between the drug chamber and the casing, the predetermined space defined by the at least one spacing element. According to some embodiments, the casing is elastic. In further embodiments, the casing is configured to be compressed against the drug chamber, thereby sealing the at least one opening. According to some embodiments, the casing comprises a perforated or porous material, the material configured to allow the pharmaceutical composition to be released from the capsule via the at least one opening and the casing.

According to some embodiments, the casing comprises at least one casing opening adapted to enable the insertion of the capsule into the casing for the utilization thereof. In further embodiments, the casing further comprises at least one elongated member configured to seal at least a portion of said at least one casing opening after the insertion thereof.

According to some embodiments, the capsule further comprises a barrier circumferentially surrounding the at least one opening.

In some embodiments, the geometry of the capsule is selected from a barrel-like shape, sphere, ellipsoid, a cylinder, and combinations thereof.

According to another aspect, there is provided a capsule configured for oral administration and drug release at a desired location in the gastrointestinal (GI) tract, comprising: at least one magnet configured to output a magnetic field external to the capsule; at least one drug chamber exhibiting at least one opening located at a surface of the at least one drug chamber; at least one pharmaceutical composition positioned within the at least one drug chamber; and a casing encompassing at least the drug chamber, wherein a predetermined space is defined between the casing and the drug chamber.

According to some embodiments, the capsule further comprises a spacing element positioned between the drug chamber and the casing, the predetermined space defined by the spacing element.

According to some embodiments, the casing is elastic.

According to some embodiments, the casing is configured to be compressed against the drug chamber, thereby sealing the at least one opening.

According to some embodiments, the casing comprises a perforated or porous material, the material configured to allow the pharmaceutical composition to be released from the capsule via the at least one opening and the casing.

According to some embodiments, the capsule comprises a plurality of magnets.

In some embodiments, the casing comprises at least one casing opening adapted to enable the insertion of the capsule into the casing for the utilization thereof. In further embodiments, the casing further comprises at least one elongated member configured to seal at least a portion of said at least one casing opening after the insertion thereof.

According to some embodiments, the capsule further comprises at least one opening cover; and at least one metallic element attached to the at least one opening cover, wherein the at least one opening cover is configured to seal the at least one opening responsive to the at least one metallic element being magnetized. According to further embodiments, the capsule further comprises at least one hinge, the at least one opening cover connected to the at least one opening via the at least one hinge, wherein the at least one hinge is configured to allow the at least one opening cover to open into the at least one drug chamber.

According to some embodiments, the capsule further comprises a barrier circumferentially surrounding the at least one opening.

In some embodiments, the geometry of the capsule is selected from a barrel-like shape, sphere, ellipsoid, a cylinder, and combinations thereof.

According to another aspect, there is provided a capsule configured for oral administration and drug release at a desired location in the gastrointestinal (GI) tract, comprising: at least one magnet configured to output a magnetic field external to the capsule; at least one drug chamber exhibiting at least one opening located at a surface of the at least one drug chamber; at least one pharmaceutical composition positioned within the at least one drug chamber; at least one opening cover; and at least one metallic element attached to the at least one opening cover, wherein the at least one opening cover is configured to seal the at least one opening responsive to the at least one metallic element being magnetized.

According to some embodiments, the capsule further comprises at least one hinge, the at least one opening cover connected to the at least one opening via the at least one hinge, wherein the at least one hinge is configured to allow the at least one opening cover to open into the at least one drug chamber.

According to some embodiments, the capsule further comprises a barrier circumferentially surrounding the at least one opening.

In some embodiments, the geometry of the capsule is selected from a barrel-like shape, sphere, ellipsoid, a cylinder, and combinations thereof.

According to another aspect, there is provided a capsule configured for oral administration and drug release at a desired location in the gastrointestinal (GI) tract, comprising: at least one magnet configured to output a magnetic field external to the capsule; at least one drug chamber exhibiting at least one opening located at a surface of the at least one drug chamber; at least one pharmaceutical composition positioned within the at least one drug chamber; and a barrier circumferentially surrounding the at least one opening.

According to another aspect, there is provided a drug delivery system comprising: a capsule as disclosed herein above; and an external magnet kit comprising at least one external magnet configured to be juxtaposed with the skin of a user at the vicinity of the desired location of the GI tract, wherein a flow of pharmaceutical composition through the at least one opening is controlled responsive to a direction of the magnetic field of the at least one external magnet. According to further embodiments, the at least one magnet is stationary within the capsule, and wherein responsive to a change in the direction of the magnetic field of the at least one external magnet, the at least one stationary magnet is configured to rotate the capsule

According to some embodiments, the drug delivery system further comprises a rail, the at least one external magnet configured to roll along the rail, wherein the direction of the magnetic field of the at least one external magnet is responsive to the roll.

According to some embodiments, the drug delivery system further comprises at least one intermediate housing; and at least one inner magnet, disposed within the at least one intermediate housing, wherein the at least one intermediate housing is configured to be implanted below the skin of the user, between the at least one external magnet and the desired location of the GI tract. According to further embodiments, the at least one intermediate housing comprises: a magnetic shield member; and a translation member, the translation member configured to translate the at least one inner magnet between a first position and a second position in relation to the magnetic shield member, wherein, responsive to the magnetic shield member, the intensity of a magnetic field of the at least one inner magnet is greater in the first position than in the second position.

According to some embodiments, the drug delivery system further comprises at least one flux limiting component configured to attenuate the magnetic field of the at least one external magnet in a direction facing away from the capsule. According to further embodiments, the at least one flux limiting component comprises at least one electromagnet. According to still further embodiments, the at least one flux limiting component comprises a plurality of electromagnets.

According to some embodiments, the drug delivery system further comprises a rotating member, the rotating member configured to rotate the at least one external magnet around a central axis of the at least one external magnet, wherein the direction of the magnetic field of the at least one external magnet is responsive to the rotation about the central axis. According to further embodiments, the at least one external magnet comprises a plurality of external magnets and the rotating member comprises a plurality of gears and an actuator configured to rotate each one of the plurality of external magnets.

According to some embodiments, the drug delivery system further comprises a control system configured to control the operation of at least one of the various components of the system.

According to some embodiments, the drug delivery system further comprises at least 3 sensors configured to detect magnetic fields; and a controller in communication with the at least 3 sensors, wherein the controller is configured, responsive to an output of the at least 3 sensors, to identify a location of the capsule.

According to another aspect, there is provided a drug delivery system comprising: a capsule configured for oral administration and drug release at a desired location in the gastrointestinal (GI) tract; and an external magnet kit comprising at least one external magnet configured to be juxtaposed with the skin of a user at the vicinity of the desired location of the GI tract, wherein the capsule comprises: at least one magnet configured to output a magnetic field external to the capsule; at least one drug chamber exhibiting at least one opening located at a surface of the at least one drug chamber; and at least one pharmaceutical composition positioned within the at least one drug chamber, wherein the geometry of the capsule is selected from a barrel-like shape, sphere, ellipsoid, a cylinder, and combinations thereof, and wherein a flow of pharmaceutical composition through the at least one opening is controlled responsive to a direction of the magnetic field of the at least one external magnet.

According to some embodiments, the at least one magnet is stationary within the capsule, and wherein responsive to a change in the direction of the magnetic field of the at least one external magnet, the at least one stationary magnet is configured to rotate the capsule.

According to some embodiments, the drug delivery system further comprises a rail, the at least one external magnet configured to roll along the rail, wherein the direction of the magnetic field of the at least one external magnet is responsive to the roll.

According to some embodiments, the drug delivery system further comprises at least one intermediate housing; and at least one inner magnet, disposed within the at least one intermediate housing, wherein the at least one intermediate housing is configured to be implanted below the skin of the user, between the at least one external magnet and the desired location of the GI tract.

According to some embodiments, the at least one intermediate housing comprises: a magnetic shield member; and a translation member, the translation member configured to translate the at least one inner magnet between a first position and a second position in relation to the magnetic shield member, wherein, responsive to the magnetic shield member, the intensity of a magnetic field of the at least one inner magnet is greater in the first position than in the second position.

According to some embodiments, the drug delivery system further comprises at least one flux limiting component configured to attenuate the magnetic field of the at least one external magnet in a direction facing away from the capsule. According to further embodiments, the at least one flux limiting component comprises at least one electromagnet.

According to still further embodiments, the at least one flux limiting component comprises a plurality of electromagnets. According to some embodiments, the at least one flux limiting component comprise a flux limiting cover.

According to some embodiments, the drug delivery system further comprises a rotating member, the rotating member configured to rotate the at least one external magnet around a central axis of the at least one external magnet, wherein the direction of the magnetic field of the at least one external magnet is responsive to the rotation about the central axis. According to further embodiments, the at least one external magnet comprises a plurality of external magnets and the rotating member comprises a plurality of gears and an actuator configured to rotate each one of the plurality of external magnets.

According to some embodiments, the drug delivery system further comprises a control system configured to control the operation of at least one of the various components of the system.

According to another aspect, there is provided a system configured to locate a desired location of the GI tract in which a pharmaceutical composition is to be released, the system comprising: a capsule configured for oral administration comprising at least one magnet configured to output a magnetic field external to the capsule; at least 3 sensors configured to detect magnetic fields; and a controller in communication with the at least 3 sensors, wherein the controller is configured, responsive to an output of the at least 3 sensors, to identify a location of the capsule within the GI tract

According to another aspect, there is provided a method for release of a pharmaceutical composition, the method comprising: providing a drug delivery system as disclosed herein above; juxtaposing the at least one external magnet to skin of a user in the vicinity of a desired location of the gastrointestinal (GI) tract; administering, per os, the capsule; securing the administered capsule to the desired location of the GI tract responsive to a magnetic field of the at least one external magnet; and repeatedly adjusting a direction of the magnetic field of the at least one external magnet thereby shaking the capsule, in order to accelerate the release of the pharmaceutical composition from the at least one opening.

According to another aspect, there is provided a method for release of a pharmaceutical composition, the method comprising: juxtaposing at least one external magnet to skin of a user in the vicinity of a desired location of the gastrointestinal (GI) tract; administering, per os, a capsule, the capsule comprising at least one magnet and at least one drug chamber, the at least one drug chamber comprising at least one opening located at a surface of the at least one drug chamber and at least one pharmaceutical composition; securing the administered capsule to the desired location of the GI tract responsive to a magnetic field of the at least one external magnet; and repeatedly adjusting a direction of the magnetic field of the at least one external magnet thereby shaking the capsule, in order to accelerate the release of the pharmaceutical composition from the at least one opening.

According to some embodiments, the method further comprises repeatedly rotating the at least one external magnet alternately in a clockwise direction and a counter-clockwise direction to cause the adjusting of the direction of the magnetic field of the at least one external magnet.

According to some embodiments, the method further comprises adjusting the direction of the magnetic field of the at least one external magnet such that the at least one opening is pressed against a wall of the GI tract and is sealed thereby.

According to some embodiments, the method further comprises prior to administering, per os, the capsule, the steps of: a) providing a device configured to measure the position and orientation of a permanent magnet within a three-dimensional region of the GI tract, the device comprising: fastening means configured to attach the device to the body of a user; at least three sensors configured to detect magnetic fields; and a controller configured to receive magnetic field data from the at least three sensors and to transmit said data to a processing unit, wherein the at least three sensors and the controller are connected to the fastening means, and wherein the processing unit is configured to determine the position and the orientation of the permanent magnet along a three-dimensional region of the GI tract; b) administering, per os, a capsule, wherein said capsule is configured for oral administration and comprise at least one permanent magnet disposed within; c) determining the position and the orientation of the capsule comprising the permanent magnet (from step b)) along a plurality of corresponding three-dimensional segments along the GI tract; d) tracking the route of the capsule comprising the permanent magnet through the corresponding three-dimensional segments along the GI tract; and e) comparing the determined position of the capsule to the desired location in the GI tract in which the pharmaceutical composition is to be release; and f) outputting an output of the comparison.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following embodiments and aspects thereof are described and illustrated in conjunction with systems, tools and methods which are meant to be exemplary and illustrative, but not limiting in scope. In various embodiments, one or more of the above-described problems have been reduced or eliminated, while other embodiments are directed to other advantages or improvements.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

In the Figures:

FIG. 1 constitutes a cross-sectional view of capsule 101 (disclosed in International Publication No. WO 2018/073726 to the inventors).

FIGS. 2A and 2B constitute cross-sectional views of drug delivery system 200, with different orientations of external magnet 240 and internal magnet 220 (disclosed in International Publication No. WO 2018/073726 to the inventors).

FIGS. 3A-3I constitute various cross-sectional views of capsule 301, according to some embodiments of the present invention.

FIG. 4 constitutes a cross-sectional view of an exemplary embodiment of drug delivery system 300 comprising capsule 301 of FIG. 3I, and an external magnet kit comprising the at least one external magnet 340.

FIGS. 5A and 5B constitute cross-sectional views of drug delivery system 300^{a}, with different orientations of external magnet 340^{a} and stationary magnet 320^{a}, according to some embodiments of the present invention.

FIGS. 6A-6D constitute various cross-sectional views of capsule 401, according to some embodiments of the present invention.

FIGS. 6E and 6F constitute cross-sectional views of capsule 401, with different orientations of capsule 401, according to some embodiments of the present invention.

FIG. 6G constitutes a cross-sectional view of capsule 401, according to some embodiments of the present invention.

FIG. 6H constitutes a side cross-sectional view of capsule 401 of FIG. 6G, according to some embodiments of the present invention.

FIG. 6I constitutes a cross-sectional view of capsule 401, according to some embodiments of the present invention.

FIG. 6J constitutes a side cross-sectional view of capsule 401 of FIG. 6I, according to some embodiments of the present invention.

FIGS. 7A and 7B constitute cross-sectional views of capsule 501, with different configurations of capsule 501, according to some embodiments of the present invention.

FIGS. 8A and 8B constitute cross-sectional views of capsule 501, with different configurations of capsule 501, according to some embodiments of the present invention.

FIGS. 9A and 9B constitutes cross-sectional views of capsule 501, with different configurations of capsule 501, according to some embodiments of the present invention.

FIGS. 10A-10E constitutes cross-sectional views of capsule 501, according to some embodiments of the present invention.

FIG. 11A constitutes cross-sectional views of capsule 601, according to some embodiments of the present invention.

FIGS. 11B and 11C constitutes cross-sectional views of capsule 601, with different configurations of capsule 601, according to some embodiments of the present invention.

FIGS. 12A and 12B constitutes cross-sectional views of capsule 601, with different configurations of capsule 601, according to some embodiments of the present invention.

FIG. 13 constitutes a cross-sectional view of drug delivery system 700, according to some embodiments of the present invention.

FIGS. 14A and 14B constitutes side views depicting different configurations of the at least one intermediate magnetic housing 745, from the perspective view of the at least one external magnet 740, according to some embodiments of the present invention.

FIG. 15 constitutes a top-view of a cross-sectional view of drug delivery system 700, according to some embodiments.

FIGS. 16-18 constitute various views in perspective of capsule 801 through different embodiments of the present invention.

FIG. 19A constitutes a cross-sectional view of drug delivery system 900, according to some embodiments of the present invention.

FIG. 19B constitutes a side view depicting different configurations of rail 941 along a partial view of fastening means 942 through different embodiments of the present invention.

FIG. 19C constitutes a side view depicting different configurations of rail 941 along a partial view of fastening means 942 through different embodiments of the present invention.

FIGS. 20A and 20B constitutes a top-view of a cross-sectional view of drug delivery system 1000, with different configurations of capsule 1001 and external magnet 1040, according to some embodiments.

FIG. 21A constitutes a cross-sectional view of capsule 1301, according to some embodiments of the present invention.

FIG. 21B constitutes a cross-sectional view of capsule 1301, according to some embodiments of the present invention.

FIG. 21C constitutes a cross-sectional view of capsule 1301, according to some embodiments of the present invention.

FIG. 22A constitutes a schematic illustration of magnetic flux in the presence of a single magnet.

FIG. 22B constitutes a schematic illustration of magnetic flux in the presence of a single magnet in the context of external magnet kit 1100, according to some embodiments of the present invention.

FIG. 23A constitutes a schematic illustration of magnetic flux in the presence of three magnets.

FIG. 23B constitutes a schematic illustration of magnetic flux in the presence of three magnets in the context of external magnet kit 1100, according to some embodiments of the present invention.

FIG. 24A constitutes a side-view of magnets 1140a-c through different embodiments of the present invention.

FIG. 24B constitutes a top-view of magnets 1140a-c held by rigid members 1148a-b, through different embodiments of the present invention.

FIG. 25 constitutes a top-view of external magnet kit 1100 through different embodiments of the present invention.

FIG. 26 constitutes a top-view of external magnet kit 1100 through different embodiments of the present invention.

FIG. 27A constitutes a top-view of external magnet kit 1100 through different embodiments of the present invention.

FIG. 27B constitutes a side-view of external magnet kit 1100 taken on line 27B-27B of FIG. 27A, through different embodiments of the present invention.

FIG. 27C constitutes a view in perspective of external magnet kit 1100, from a front-left angle, through different embodiments of the present invention.

FIG. 28 constitutes a view in perspective of external magnet kit 1100, from a front-left angle, through different embodiments of the present invention.

FIG. 29A constitutes a cross-sectional view of the GI tract region 1200, according to some embodiments.

FIG. 29B constitutes a cross-sectional view of GI tract region 1200, according to some embodiments.

### DETAILED DESCRIPTION

The present invention provides magnetically controlled capsules and drug delivery systems configured for oral administration and adapted for controllable drug release at a desired location of the GI tract, and methods of using same for treating a disease or disorder.

The term ‘drug’, as used herein, is interchangeable with the terms ‘pharmaceutical composition’, ‘pharmaceutically active agent’, ‘medication’, ‘medicament’ and synonyms thereof and refers to a compound or agent that is capable of inducing a therapeutic effect, when administered to a subject in need thereof.

The term ‘GI tract’, as used herein, refers to the gastrointestinal tract, including the stomach and the small and large intestine.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout.

Throughout the figures of the drawings, different superscripts for the same reference numerals are used to denote different embodiments of the same elements. Embodiments of the disclosed devices and systems may include any combination of different embodiments of the same elements. Specifically, any reference to an element without a superscript may refer to any alternative embodiment of the same element denoted with a superscript. In order to avoid undue clutter from having too many reference numbers and lead lines on a particular drawing, some components will be introduced via one or more drawings and not explicitly identified in every subsequent drawing that contains that component.

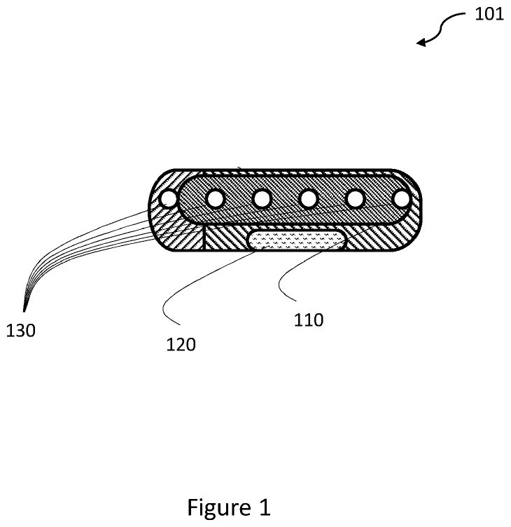

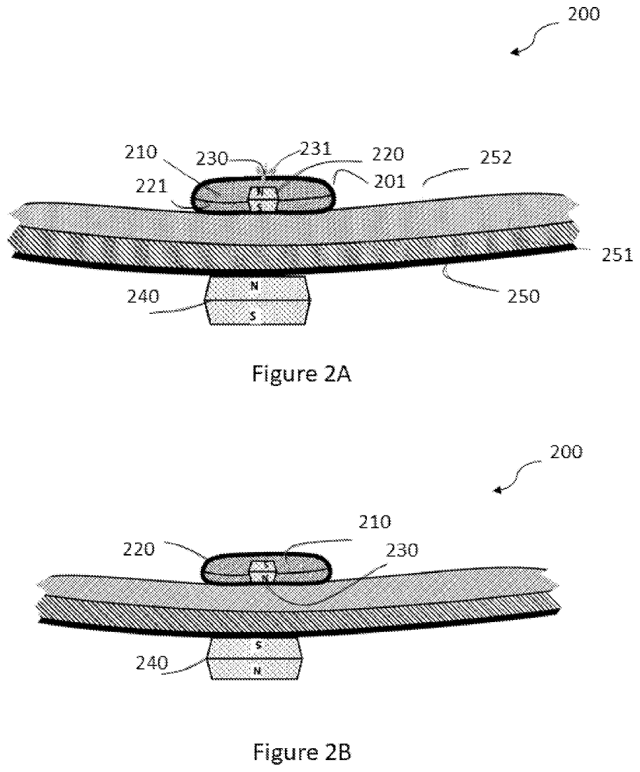

Reference is now made to FIG. 1 and FIGS. 2A-2B presenting cross-sectional views of capsule 101 and drug delivery system 200, respectively, as disclosed in International Publication No. WO 2018/073726 to the inventors, the entire contents of which incorporated herein by reference.

Capsule 101 is part of a delivery system as discussed herein. Capsule 101 is adapted to contain pharmaceutical composition 110 disposed therewithin and at least one internal permanent magnet 120, as illustrated at FIG. 1. Internal permanent magnet 120 is typically disposed within capsule 101. Capsule 101 may further comprise at least one drug flow opening 130, typically located on a surface of capsule 101. For example, capsule 101 comprises a plurality of flow openings 130 as shown in FIG. 1.

The pharmaceutical compositions disclosed herein, including pharmaceutical composition 110, may be in the form selected from solid (e.g. a pill or a tablet), gel, suspension and/or liquid.

The drug delivery system encompassing capsule 101 comprises at least one external magnet (not shown), configured to be held externally, against the skin of a user, at the vicinity of a desired location of the GI tract, as further elaborated in International Publication No. WO 2018/073726 to the inventors.

Drug delivery system 200 comprises capsule 201 adapted to contain at least one pharmaceutical composition 210 disposed therewithin, at least one internal magnet 220, at least one drug flow opening 230, and at least one membrane 221. Internal magnet 220 is attached to membrane 221 and is configured to move perpendicularly to the longitudinal axis, but not laterally along the horizontal axis of capsule 201, where membrane 221 is attached to the internal surface of capsule 201, as depicted at FIG. 2A.

Drug delivery system 200 further comprises at least one external magnet 240 configured to generate an external magnetic field. External magnet 240 is juxtaposed with the outer layer of the skin 250 of a user, at the vicinity of a desired location of the GI tract.

As illustrated in FIGS. 2A and 2B, external magnet 240 attracts internal magnet 220, and therefore secures capsule 201 at a desired location of the GI tract. Internal region 251 comprises internal tissues (such as, dermis, subcutaneous layer and so on) residing between capsule 201 and external magnet 240. The at least one external magnet 240 is configured to control drug flow from the capsule 201 into the GI tract, as a function of changes in the external magnetic field induced by the at least one external magnet 240. The at least one internal magnet 220 is configured to rotate the capsule, in response to changes in the direction of the external magnetic field.

Internal magnet 220 is configured to secure capsule 201 in contact with GI wall 252 in the vicinity of the desired location of the GI tract, by juxtaposing the south magnetic pole of internal magnet 220 with the north pole of external magnet 240, the south magnetic pole of internal magnet 220 located at the surface of capsule 201 facing GI wall 252. Under the aforementioned configuration, pharmaceutical composition 210 can be released from capsule 201 through the at least one drug flow opening 230 into the desired location of the GI tract (as depicted in FIG. 2A). Drug release direction 231 illustrates the release of pharmaceutical composition 210 into the GI tract. The release of the drug can be ceased or suspended by changing the external magnetic field induced by the at least one external magnet 240, resulting in the rotation of the capsule and accordingly the sealing of the at least one drug flow opening 230 by the GI wall 252, as depicted in FIG. 2B, and as further elaborated in International Publication No. WO 2018/073726 to the inventors.

According to a first aspect, there is provided a drug delivery system comprising a capsule and an external magnet kit.

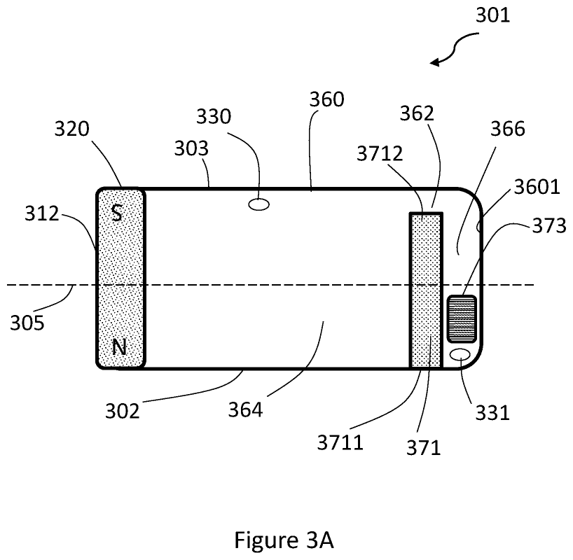

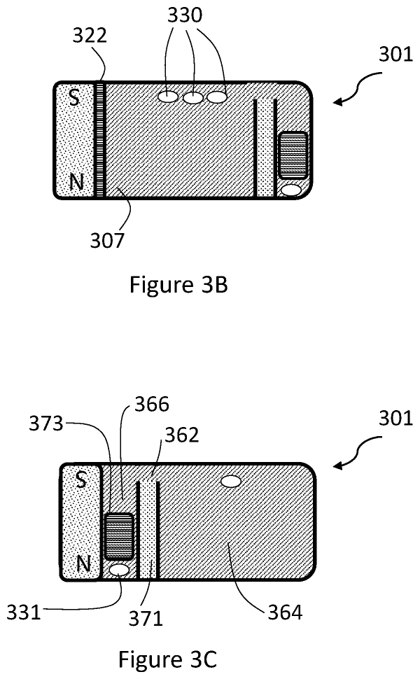

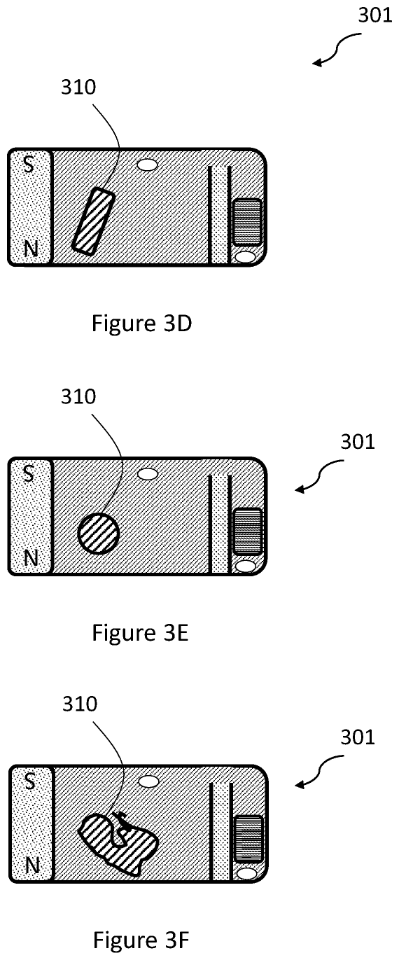

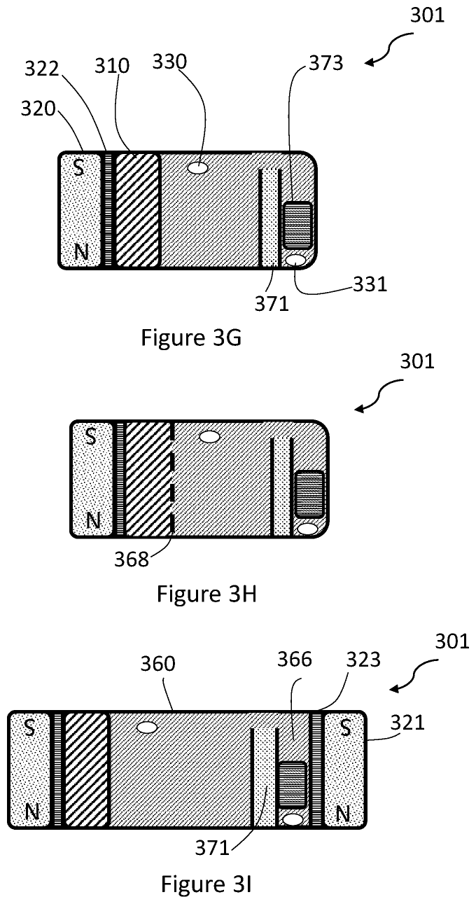

Reference is now made to FIGS. 3A-3I which constitute cross-sectional views depicting capsule 301 through different embodiments of the present invention.

According to some embodiments, capsule 301 comprises at least one stationary magnet 320, and at least one drug chamber 360, wherein the at least one drug chamber 360 comprises or exhibits at least one first opening 330 and at least one second opening 331, a mobile member 373 configured to be magnetized by an external magnetic field, and at least one partitioning element 371 positioned so as to partition the drug chamber 360 into two fluidly connected compartments. According to some embodiments, at least one partitioning element 371 is oriented perpendicularly to a central longitudinal axis 305 of capsule 301. According to some embodiments, at least one partitioning element 371 is oriented in parallel to a wall 3601 of the drug chamber 360 (e.g., the shortest face of an inner surface of the capsule 301), mobile member 373 being positioned between at least one partitioning element 371 and wall 3601. According to some embodiments, mobile member 373 is shaped and dimensioned such that mobile member 373 extends from at least one partitioning element 371 to the wall 3601. The term “mobile member”, as used herein, means a member that is mobile within capsule 301.

It is to be understood that the capsules of the delivery systems disclosed herein, such as capsule 301, are manufactured to have a size and a shape appropriate for ingestion and for easy delivery through the various segments of the GI tract.

According to some embodiments, capsule 301 comprises a first surface 302 and a second surface 303, parallelly facing each other. According to some embodiments, the at least one partitioning element 371 is perpendicularly attached to at least one of first surface 302 and second surface 303. According to some embodiments, the at least one partitioning element 371 comprises a first partitioning element end 3711 and a second partitioning element end 3712, such that the first partitioning element end 3711 is attached to a first surface 302 of capsule 301 and the second partitioning element end 3712 is spaced from a second surface 303 of capsule 301, thereby forming a passage 362 between the second partitioning element end 3712 and the second surface 303.

Partitioning element 371 partitions the at least one drug chamber 360 to a first compartment 364 comprising the first opening 330, and a second compartment 366 comprising the mobile member 373 and the second opening 331, wherein the passage 362 is configured to provide a fluid communication between said first compartment 364 and second compartment 366. In some embodiments, drug chamber 360 further comprises one or more additional partitioning elements configured to partition the drug chamber 360 into one or more additional compartments.

According to some embodiments, the premises of first compartment 364 are defined by partitioning element 371, the stationary magnet 320, first surface 302 and second surface 303. According to some embodiments, the first compartment 364 extends between partitioning element 371, in particular one surface thereof, and the stationary magnet 320 (in particular one surface thereof). According to some embodiments, the second compartment 366 occupies the remaining portion of drug compartment 360, i.e. the portion not occupied by first compartment 364. According to some embodiments, the second compartment 366 extends between partitioning element 371 (in particular the surface/side of partitioning element 371 closer to mobile member 373) and the surface of capsule 301, as presented at FIG. 3A.

According to some embodiments, the mobile member 373 is configured to move within the second compartment 366 between the first surface 302 and the second surface 303 of the capsule 301. According to some further embodiments, the mobile member 373 is configured to move perpendicularly to the central longitudinal axis 305 of capsule 301. In further embodiments, the mobile member 373 is configured to be magnetized and to move along the second compartment 366, in response to changes in direction of an external magnetic field induced by an external magnet.

According to some embodiments, drug chamber 360 is configured to contain therewithin a pharmaceutical composition. According to further embodiments, the drug chamber 360 further comprises at least one pharmaceutical composition 310, comprising at least one pharmaceutical active agent (shown in FIGS. 3D-3I).

According to some embodiments, drug chamber 360 further comprises at least one pharmaceutical unit comprising the pharmaceutical composition 310 comprising the at least one pharmaceutical active agent. According to some embodiments, the at least one pharmaceutical unit is either a pharmaceutical composition 310, for example, a solid dosage form pharmaceutical composition (such as, tablets, pills and the like) or is encapsulating a pharmaceutical composition (for example, the pharmaceutical unit is a capsule and the like). According to some embodiments, the at least one pharmaceutical unit is selected from the group consisting of a pill, a powder, a tablet, a micro tablet, a granule, a capsule, a gel, a liquid, a liquid-gel, or combinations thereof. Each possibility represents a separate embodiment of the present invention.

According to some embodiments, the at least one pharmaceutical composition 310 is in any suitable form known in the pharmaceutical art. In some embodiments, the at least one drug chamber 360 comprise a plurality of pharmaceutical compositions 310 disposed therewithin. In further embodiments, the at least one drug chamber 360 comprises a plurality of pharmaceutical units disposed therewithin.

In some embodiments, the pharmaceutical composition 310 is disposed within the drug chamber 360. The at least one pharmaceutical composition 310 can be entangled, embedded, incorporated, encapsulated, bound, or attached to an inner surface of drug chamber 360 in any way known in the art. In further embodiments, the pharmaceutical composition 310 is disposed within the first compartment 364. In further such embodiments, the pharmaceutical composition 310 is entangled, embedded, incorporated, encapsulated, bound, or attached to an inner surface of the first compartment 364 in any way known in the art. In some embodiments, the first compartment 364 comprise a plurality of pharmaceutical compositions 310 disposed therewithin.

According to some embodiments, the at least one pharmaceutical active agent is selected from the group consisting of antibiotics, antivirals, antifungals, antiangiogenics, analgesics, anesthetics, anti-inflammatory agents including steroidal and non-steroidal anti-inflammatories (NSAIDs), corticosteroids, antihistamines, mydriatics, antineoplastics, immunosuppressive agents, anti-allergic agents, metalloproteinase inhibitors, tissue inhibitors of metalloproteinases (TIMPs), vascular endothelial growth factor (VEGF) inhibitors or antagonists or intraceptors, receptor antagonists, RNA aptamers, antibodies, hydroxamic acids and macrocyclic anti-succinate hydroxamate derivatives, nucleic acids, plasmids, siRNAs, vaccines, DNA binding compounds, hormones, vitamins, proteins, peptides, polypeptides and peptide-like therapeutic agents, anesthetizers and combinations thereof. Each possibility represents a separate embodiment of the present invention.

According to one embodiment, the pharmaceutical composition 310 comprises at least one of carbidopa and levodopa. According to some embodiments, the pharmaceutical composition 310 comprises carbidopa and levodopa (a drug combination also known as “carbidopa/levodopa” or “levocarb and co-careldopa”).

According to some embodiments, there is provided a method for treating Parkinson's disease, the method comprising use of the drug delivery system(s) as disclosed herein, wherein the pharmaceutical composition 310 comprises at least one of carbidopa and levodopa.

According to some embodiments, the pharmaceutical composition 310 further comprises at least one pharmaceutical carrier. Pharmaceutical carriers that may be used in the context of the present invention include various organic or inorganic carriers including, but not limited to, excipients, lubricants, binders, disintegrants, water-soluble polymers and basic inorganic salts. The pharmaceutical compositions of the present invention may further include additives such as, but not limited to, preservatives, antioxidants, coloring agents, sweetening agents, souring agents, bubbling agents and flavorings.

According to some embodiments, the first opening 330 is located on, or embedded within, the surface of the first compartment 364, as presented at FIG. 3A. The first opening 330 can be positioned in a variety of locations along the surface of the first compartment 364. According to some embodiments, the first opening 330 is configured to provide fluid communication between said first compartment 364 and the GI tract. In some embodiments, the first opening 330 is in the shape of a circle, ellipse, square, rectangle, or any other polygon.

The terms “located on” and “embedded within” as used herein collectively refer to the configuration of the openings disclosed herein (e.g. 330 and 331) with respect to the surface of the capsule. The opening may be formed as an integral part of the surface, or generated on the surface (e.g. by puncturing or melting, for example, using laser) such that the resulting opening at the surface is preferably forming a continuous smooth and seamless surface with one or more openings.

In some embodiments, the first opening 330 comprises a plurality of openings. According to some embodiments, the openings are aligned on the surface of the first compartment 364. According to some embodiments, the plurality of openings is scattered on the surface of the first compartment 364. According to some embodiments, each of opening 330 is in the shape of a circle and has a diameter in the range of about 0.1 mm to 5 mm, about 0.5 mm to 4 mm, about 0.5 mm to 3.5 mm, about 0.5 mm to 3 mm, about 0.5 mm to 2.5 mm, about 0.5 mm to 2 mm, about 0.5 mm to 1.5 mm or about 0.5 mm to 1 mm. Each possibility represents a separate embodiment.

According to some embodiments, the at least one drug chamber 360 comprises a plurality of first openings 330. According to some embodiments, the plurality of first openings 330 are aligned on the surface of the at least one drug chamber 360. According to some embodiments, the plurality of first openings 330 is scattered on the surface of the at least one drug chamber 360.

It is noted that the same functional and structural principles disclosed for first opening 330 similarly apply to the second opening 331 which is located on, or embedded within, the surface of the second compartment 366, as presented at FIG. 3A.

According to some embodiments, capsule 301 comprises an external housing 312 comprising the first surface 302 and the second surface 303 on opposite sides of capsule 301 (e.g. top and bottom or left and right relative to one another). According to some embodiments, external housing 312 is configured to enable capsule 301 to pass through the GI tract without dissolving or degrading in an undesired location.

According to some embodiments, external housing 312 comprises a durable and biocompatible material. According to some embodiments external housing 312 is consisting of a durable and biocompatible material. According to some embodiments external housing 312 is formed of a durable and biocompatible material, configured to enable capsule 301 to pass through the GI tract without dissolving or degrading in an undesired location. According to some embodiments, said durable and biocompatible material is selected from a biocompatible polymer, such as but not limited to: polyethylene glycol (PEG), polylactic acid (PLA), polymethylmethacrylate, polyethylmethacrylate, polybutylmethacrylate, poly-2-ethylhexylmethacrylate, polylaurylmethacrylate, polyhydroxylethyl methacrylate, polymethylacrylate, 2-methacryloyloxyethylphosphorylcholine (MPC), polystyrene, poly n-ethyl-4-vinyl-pyridinium bromide, polyvinyl acetate, and derivatives and/or combinations thereof. Each possibility represents a separate embodiment of the present invention.

According to some embodiments, said durable and biocompatible material comprises an enteric coating. It is to be understood that external housing 312 can be made of any material known in the art that enables capsule 301 to pass through the GI tract without dissolving or degrading in an undesired location

The term “enteric coating” as used herein, refers to a polymer material capable of preventing dissolution or disintegration of capsule 301 in the acidity of the environment of the stomach. In some embodiments, the enteric coating is selected from: methyl acrylate, methyl methacrylate, cellulose acetate phthalate (CAP), cellulose acetate succinate, hydroxypropyl methyl cellulose phthalate, hydroxypropyl methyl cellulose acetate succinate, polyvinyl acetate phthalate (PVAP), cellulose acetate trimellitate, shellac, alginic acid, zein, and derivatives and/or combinations thereof. Each possibility represents a separate embodiment of the present invention.

Capsule 301 is manufactured to have a size and a shape appropriate for ingestion and for easy delivery through the various segments of the GI tract. In some embodiments, the length of the capsule 301 in its longest dimension is within the ranges of 0.3 cm to 3 cm. In some embodiments, the length of the capsule 301 in its longest dimension is within the range of 2.5 cm to 3 cm, from 2 cm to 2.5 cm, from 1.5 cm to 2 cm, from 1 cm to 1.5 cm, from 0.5 cm to 1 cm or from 0.3 cm to 0.5 cm. Each possibility represents a separate embodiment of the present invention. In further embodiments, the length of the capsule in its longest dimension is within the ranges of 1 cm to 2.5 cm.

In some embodiments, the length of the capsule 301 in its shortest dimension is within the ranges of 0.3 cm to 1.5 cm. In some embodiments, capsule 301 is in the shape of a cylinder having hemispherical ends. In some embodiments, the diameter of a cross-section of the cylinder is within the ranges of 0.3 cm to 1.5 cm, 0.5 to 1.5 cm, 0.5 to 1.0 cm or 0.3 cm to 0.5 cm. Each possibility represents a separate embodiment of the present invention. In some exemplary embodiments, the diameter of a cross-section of the cylinder is within the ranges of 0.3 cm to 1.5 cm.

According to some embodiments, the mobile member 373 comprises a ferromagnetic compound, such as but not limited to, soft iron. According to some embodiments, the mobile member 373 comprises at least one component selected from the group consisting of a rare earth metal, a naturally occurring mineral, or any combination thereof. In some embodiments, the ferromagnetic compound is selected from: iron, iron oxide, neodymium iron boron, nickel, aluminum nickel cobalt, cobalt, samarium cobalt, and any combination thereof. Each possibility represents a separate embodiment of the present invention. In some embodiments, the rare earth metal is selected from scandium, yttrium, lanthanide, and any combination thereof. Each possibility represents a separate embodiment of the present invention. The naturally occurring mineral can be lodestone. In some exemplary embodiments, the mobile member 373 contain a ferromagnetic compound comprising soft iron.

According to some embodiments, the mobile member 373 is configured to be shaped as a sphere, a cylinder, a disc, or any polyhedron thereof. In some embodiments, the diameter of mobile member 373 is smaller than the shortest dimension of the second compartment 366, thereby mobile member 373 can easily move within the second compartment 366, if/when required.

It is to be understood that the movement of a mobile member, such as, mobile member 373, within the second compartment, such as second compartment 366, is primarily induced by the magnetic field generated by the external magnet, when the delivery system is assembled. Structural elements, such as, spring(s), sponge(s), wire(s), membrane-like structures, or electrical component(s), including a pump or an engine are not included in the mechanism which induces movement of the mobile member within the second compartment. Advantageously, the lack of structural elements or electrical components contributes for the cost effective and/or simple manufacture of a light weight capsule configured for magnetically controlled drug delivery. Similarly, it is to be understood that all of the capsules of the present invention as disclosed herein further below are devoid of any electrical components.

According to some embodiments, the drug chamber 360 comprises a plurality of compartments, wherein at least one compartment is a drug compartment adapted to contain a pharmaceutical composition therein. It is to be understood that the drug compartment 360 may be filled with a pharmaceutical composition at the time of administration, prior to administration, and when being consumed by a subject but prior to its release from the capsule 301 at the desired location in the GI tract.

According to some embodiments, the at least one stationary magnet, such as stationary magnet 320, is a permanent magnet, such as, but not limited to, ceramic, ferrite or

Alnico magnets. In some embodiments, the stationary magnet comprises at least one component selected from the group consisting of a ferromagnetic compound, a rare earth metal, a naturally occurring mineral, and any combination thereof. Each possibility represents a separate embodiment of the present invention. In some embodiments, the ferromagnetic compound is selected from: iron, iron oxide, neodymium iron boron, nickel, aluminum nickel cobalt, cobalt, samarium cobalt, and any combination thereof. Each possibility represents a separate embodiment of the present invention. In some embodiments, the rare earth metal is selected from scandium, yttrium, lanthanide, and any combination thereof. Each possibility represents a separate embodiment of the present invention. The naturally occurring mineral can be lodestone.

According to some embodiments, the at least one stationary magnet 320 comprises a corrosion-resistant coating. Said coating may comprise at least one material selected from a metal (such as but not limited to nickel, copper, gold, silver, zinc, and tin), a polymer (such as but not limited to parylene, polytetrafluoroethylene (PTFE), and epoxy), various paints, lacquers, and combinations thereof.

According to some embodiments, the stationary magnet 320 is cylinder shaped. However, it is to be understood that the stationary magnet 320 fulfills the same function when otherwise shaped, as a disc, sphere, ring, ovoid, ellipsoid, or any other polyhedron. Each possibility represents a separate embodiment of the present invention. In some embodiments, the cross-sectional shape of the stationary magnet 320 is dome-shaped. It is to be understood, however, that the cross-sectional geometry of stationary magnet 320 may be of a different shape, such as a circular, triangular or any other curvilinear or rectilinear cross-section. Each possibility represents a separate embodiment of the present invention.

According to some embodiments the dimensions of the stationary magnet, such as stationary magnet 320, are designed such that the stationary magnet fits within the capsule, e.g. within capsule 301.

Stationary magnet 320 can be axially magnetized or diametrically magnetized.

The term “axially magnetized or “axial direction of magnetic polarity” as used herein, are interchangeable, and refers to a magnet optionally shaped as a cylinder, ring or a disc being magnetized generally along its longitudinal axis. In this configuration, the magnetization direction is generally along the central longitudinal axis of the magnet (e.g., axis 305), and the north and south poles of the magnet are located at the edges/poles of the cylinder, on the outer surfaces of the cylinder or the disc.

The terms “diametrically magnetized” or “diametrical direction of magnetic polarity” as used herein, are interchangeable, and refers to a magnet optionally shaped as a cylinder, ring or a disc being magnetized generally across its diameter along its horizontal axis. In this configuration, the magnetization direction is generally perpendicular to the central longitudinal axis of the magnet (e.g., axis 305), and the north and south poles of the magnet are located on the outer curved surface, at opposite sides.

In some embodiments, the stationary magnet 320 is disposed within an inner surface of the capsule 301 and is stationary therein. In other embodiments, the stationary magnet 320 is disposed within the drug chamber 360.

According to some embodiments, the stationary magnet 320 is configured to have a diametrical direction of magnetic polarity. In further embodiments, the stationary magnet 320 is located perpendicularly to the central longitudinal axis 305 of the capsule 301. In still further embodiments, stationary magnet 320 is disposed within an inner surface of the capsule 301, perpendicularly to the central longitudinal axis 305 of capsule 301, as shown at FIG. 3A. In yet still further embodiments, stationary magnet 320 is in contact with, or attached to, the shortest face of the inner surface of the capsule 301, perpendicularly to the central longitudinal axis 305 of capsule 301.

In some other embodiments, the stationary magnet 320 is in contact with, or attached to, the shortest face of the external surface 312 of capsule 301, perpendicularly to the central longitudinal axis 305.

According to some embodiments, the stationary magnet is configured to be held within the capsule such that it is substantially fixed in its position, and maintains its position throughout the use of the system disclosed herein. The stationary magnet may be fixed by attachment or contact. Attachment may involve the application of suitable materials, such as, glue, resins, pastes and the like, which are preferably biocompatible. The stationary magnet may be attached to the surface of the capsule through a fastening mechanism such as but not limited to, snap-fit fastener mechanism, bayonet mount, screw fittings, or any structural mechanism similar thereto.

According to some embodiments, capsule 301 is configured for oral administration. In some embodiments, capsule 301 is configured for magnetically controlled drug release at a desired location of the GI tract. Said desired location in the GI tract can be selected from the duodenum, the jejunum and the ileum.

FIG. 3B illustrates yet another embodiment of the capsule 301 which differs from the embodiments of FIG. 3A in that the capsule 301 further comprises at least one magnetic shield 322, wherein said magnetic shield 322 is adapted to separate the at least one stationary magnet 320 from the drug chamber 360, as shown at FIG. 3B. Additionally, capsule 301 further comprises a plurality of first openings 330, wherein said openings are aligned on the surface of the first compartment 364.

According to some embodiments, said magnetic shield 322 is positioned in parallel to stationary magnet 320 and hence perpendicularly to the central longitudinal axis 305 of capsule 301. In further embodiments, capsule 301 comprise a plurality of stationary magnets 320 and a corresponding plurality of magnetic shields 322. In still further embodiments, the at least one magnetic shield 322 is adapted to separate the at least one mobile member 373 from a magnetic field induced by the stationary magnet 320.

According to some embodiments, the at least one stationary magnet 320 is configured to generate/output a magnetic field. In order to prevent the magnetic field of the at least one stationary magnet 320 from affecting the mobile member 373, the magnetic shield 322 is positioned between the at least one stationary magnet 320 and the mobile member 373.

Without wishing to being bound by any theory or mechanism of action, it is contemplated that the magnetic shield 322 is configured to shield the mobile member 373 from the magnetic field generated by the at least one stationary magnet 320. In accordance with the principles of the present invention, the mobile member 373 is configured to respond to changes in an external magnetic field, generated by an external magnet, wherein said external magnet is positioned outside the body of a user. In some embodiments, magnetic shield 322 is configured to limit or reduce the magnetic field generated by at least one stationary magnet 320 in the direction of the mobile member 373, thus allowing the mobile member 373 to operate without internal interferences. In some embodiments, the magnetic shield 322 is configured to isolate the mobile member 373 from the magnetic field generated by at least one stationary magnet 320.

According to some embodiments, the magnetic shield 322 comprise a material selected from the group consisting of sheet metal, metal screen, and metal foam. The metal can be selected from copper, nickel, stainless steel, aluminum, bras, and combinations thereof.

In some embodiments, the drug chamber further contains a pharmaceutically acceptable carrier fluid 307. According to some embodiments, the pharmaceutically acceptable carrier fluid 307 is inert towards the pharmaceutical unit and/or the pharmaceutical composition 310 contained therein. In further embodiments, the pharmaceutically acceptable carrier fluid 307 does not induce undesired interactions with the pharmaceutical unit and/or the pharmaceutical composition 310 contained therein, for example, fluid 307 does not dissolve the pharmaceutical composition 310 or its contents.

As used herein, the terms “pharmaceutically acceptable carrier fluid”, “pharmaceutically acceptable carrier liquid”, “carrier fluid” or “carrier liquid” are interchangeable, and refers to carriers which are recognized by the FDA as being safe for use in humans. Suitable carriers may be selected from water, organic solvents, cellulose derivatives, alcohols, antioxidants (such as vitamin E, vitamin C, etc.) anti-microbials (parabens, etc.), pH adjusting agents (sodium hydroxide, hydrochloric acid, etc.), tonicity adjusting agents (sodium chloride, etc.), viscosity adjusting agents (polyvinyl pyrrolidone, etc.), derivatives and combinations thereof.

It is to be understood, that the release of pharmaceutical unit and/or the pharmaceutical composition 310 contained therein to the GI tract, may require dissolution of the pharmaceutical unit and/or the pharmaceutical composition 310 contained therein, however, the dissolution is induced by liquids from the GI tract that diffuse into the capsule.

According to some embodiments, fluid 307 is designed to prevent asymmetric aggressive diffusion of liquids from the GI tract into the capsule, which may cause undesired deformation, or even destruction, of capsule 301.

As used herein, the terms “fluid from the GI tract” or “liquids from the GI tract” are interchangeable and refer to the gastrointestinal fluid residing in the GI tract. Said fluid can contain substances or fluids from the stomach and/or the intestines, such as but not limited to, partially digested food, water, acids, digestive enzymes, bile from the liver, and combinations thereof.

The pharmaceutically acceptable carrier fluid 307 can be used to enable diffusion therethrough, thereby achieving equalized concentrations between the interior of the first compartment 364 and the GI tract. The term “diffusion” as used herein, refers to the movement of substances from a region of higher concentration to a region of lower concentration, driven by a gradient in concentration.

FIG. 3C illustrates yet another embodiment of the capsule 301 which differs from the embodiments of FIG. 3A in that partitioning element 371 partitions the at least one drug chamber 360 to the first compartment 364, wherein the first compartment 364 extends between one side of partitioning element 371, in particular one surface thereof, and the surface of capsule 301, and a second compartment 366, wherein the second compartment 366 extends between the other side of partitioning element 371, in particular one surface thereof, and the stationary magnet 320.

FIG. 3D illustrates yet another embodiment of the capsule 301 which differs from the embodiments of FIG. 3A in that capsule 301 further comprises at least one pharmaceutical unit, wherein said pharmaceutical unit is disposed within the first compartment 364, wherein the dimensions of the openings (e.g. opening 330 and opening 331) are adapted to prevent the premature departure of the pharmaceutical unit therethrough, and wherein the pharmaceutical unit is encapsulating, or otherwise containing, a pharmaceutical composition 310 therein. According to some embodiments, the pharmaceutical unit is not attached to a specific inner surface of the first compartment 364, yet it may occasionally contact any one of the inner surfaces of the capsule, while freely moving in drug chamber 360. According to some embodiment, the pharmaceutical unit is a dosage form of suppository, liquid-gel or capsule, and the like.

FIG. 3E illustrates yet another embodiment of the capsule 301 which is similar to FIG. 3D, except that the pharmaceutical unit is formulated in a solid dosage form of a pharmaceutical composition 310, such as a pill, tablet, granules and globules among others.

FIG. 3F illustrates yet another embodiment of the capsule 301 which differs from the embodiments of FIGS. 3D and 3E in that the pharmaceutical composition 310 is formulated as a semi-solid dosage form, such as, a gel.

According to some embodiments, the first compartment 364 further comprises a permeable membrane (not shown) partitioning the first compartment 364 into two portions: a first portion adapted to contain the pharmaceutical composition 310 according to FIG. 3F, and a second portion adapted to contain a pharmaceutically acceptable carrier liquid 307.

According to some embodiments, the permeable membrane covers, or otherwise envelopes or wraps, the semi-solid pharmaceutical composition 310. In some embodiments, the permeable membrane is configured to prevent transition of the pharmaceutical composition 310 from the first portion into the second portion. In further embodiments, the permeable membrane is configured to allow fluids to enter from the second portion into the first portion and vice versa.

According to some embodiments, the permeable membrane enables diffusion and/or osmosis, therethrough thereby enabling to achieve equalized concentrations between the first portion and the second portion. According to some embodiments, the permeable membrane enables diffusion and/or osmosis therethrough thereby enabling to achieve equalized concentrations between the first chamber 364 and the semi-solid pharmaceutical composition 310, according to FIG. 3F.

According to some embodiments, the membrane is designed to prevent asymmetric diffusion of liquids between the semi-solid pharmaceutical composition 310 and the drug chamber 360.

According to some embodiments, the membrane is designed to prevent leakage of the at least one pharmaceutical active agent contained within the semi-solid pharmaceutical composition 310 to the drug chamber 360.

The capsule 301 is configured to allow fluid from the GI tract to flow into the first compartment 364 through the first opening 330. In some embodiments, the permeable membrane is configured to allow fluid from the GI tract to enter from the second portion into the first portion, to dissolve the pharmaceutical composition 310, and to exit from the first portion into the second portion. In further embodiments, the dissolved pharmaceutical composition 310 releases pharmaceutical active agents by diffusion into the second portion.

In some embodiments, the permeable membrane comprise a polymeric material selected from the group consisting of: cellulose acetate (CA), nitrocellulose (CN), cellulose esters (CE), polysulfone (PS), polyether sulfone (PES), polyacrylonitrile (PAN), polyamide, polyimide, polyethylene (PE), polypropylene (PP), polytetrafluoroethylene (PTFE), polyvinylidene fluoride (PVDF), polyvinylchloride (PVC), and derivatives and combinations thereof. Each possibility represents a separate embodiment of the present invention.

FIG. 3G illustrates yet another embodiment of the capsule 301 which differs from the embodiments of FIG. 3A in that capsule 301 further comprises: at least one magnetic shield 322; and at least one pharmaceutical composition 310, wherein said pharmaceutical composition 310 is extending between the first surface 302 and the second surface 303 of capsule 301, and is attached to said magnetic shield 322 at one end and is facing the first compartment 364 at the other end.

FIG. 3H illustrates yet another embodiment of the capsule 301 which differs from the embodiments of FIG. 3G in that capsule 301 further comprises a permeable membrane 368 partitioning the first compartment 364 into two portions: a first portion adapted to contain the pharmaceutical composition 310, and a second portion adapted to contain the pharmaceutically acceptable carrier fluid 307, as was presented herein above.

It is noted that the same functional and structural principles disclosed for the permeable membrane disclosed at FIG. 3F similarly apply to the permeable membrane 368 as presented at FIG. 3H.

FIG. 3I illustrates yet another embodiment of the capsule 301 which differs from the embodiments of FIG. 3G in that: the at least one stationary magnet 320 comprises a first stationary magnet 320 and a second stationary magnet 321, and the at least one magnetic shield 322 comprises a first magnetic shield 322 and a second magnetic shield 323

In some embodiments, said second magnetic shield 323 separates the second stationary magnet 321 from the drug chamber 360. In further embodiments, the second stationary magnet 321 is attached to the second magnetic shield 323 on one end.

In some embodiments, the second stationary magnet 321 is disposed within an inner surface of the capsule 301, parallelly facing the first stationary magnet 320, as shown at FIG. 3I. In further embodiments, the second stationary magnet 321 is in contact with, or attached to, the shortest face of the inner surface of the capsule 301, perpendicularly to the central longitudinal axis 305 of capsule 301, parallelly facing the first stationary magnet 320.

It is noted that the same functional, structural and operational principles disclosed herein for the at least one magnetic shield 322 and at least one stationary magnet 320, similarly apply to the second magnetic shield 323 and the second stationary magnet 321, respectively.

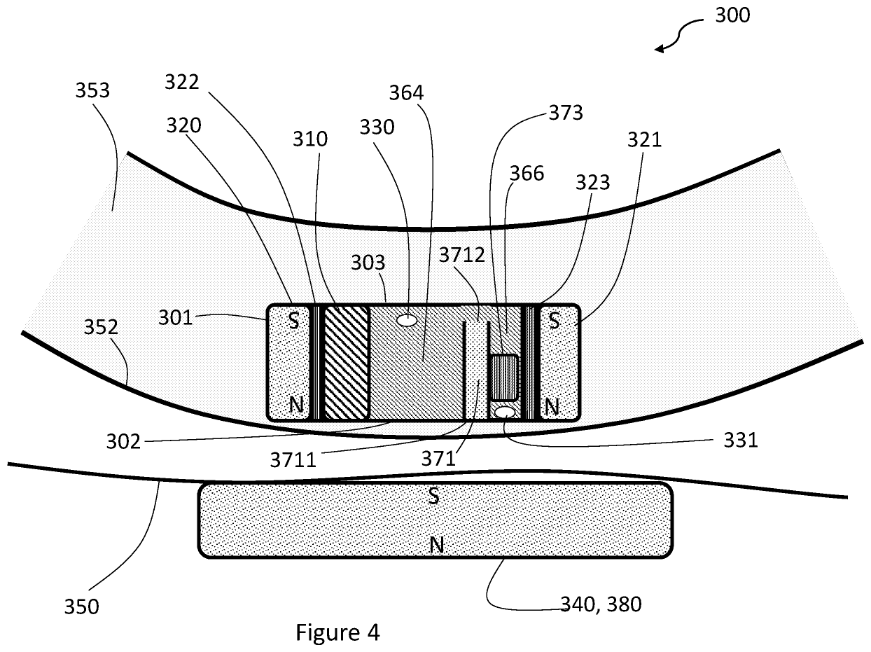

Reference is now made to FIG. 4. FIG. 4 constitutes a cross-sectional view of an exemplary embodiment of drug delivery system 300 comprising the capsule 301 of FIG. 3I, and an external magnet kit (not shown) comprising the at least one external magnet 340.

In some embodiments, the at least one external magnet 340 is configured to generate an external magnetic field. In some embodiments, the at least one external magnet 340 is configured to be in contact and/or juxtaposed with the skin of a user, located externally thereto, at the vicinity of the desired location of the GI tract.

As used herein, the term “in contact with the skin of a user” can refer to direct contact or to indirect contact, i.e. contact through at least one layer of clothing.

As used herein, the term “vicinity of the desired location” refers to an area generally surrounding the desired location of the GI tract in which the pharmaceutical composition is to be released. According to some embodiments, said area is located along the skin of the user, generally parallel to the direction or orientation of the desired location in the GI tract. According to some embodiments, said area is located inside the GI tract, optionally within a 10 cm radius, or preferably within a 5 cm radius from the desired location of the GI tract in which the pharmaceutical composition is to be released.

According to some embodiments, the magnetic field intensity of the at least one external magnet 340 is higher than the magnetic field intensity within the capsule. Without wishing to being bound by any theory or mechanism of action, it is contemplated that the magnetic field generated by the at least one external magnet 340 is higher than the magnetic field generated by the at least one stationary magnet 320, in order for the at least one external magnet 340 to be able to control the flow of the pharmaceutical active agents from the capsule to the GI tract, and to change the position of mobile member 373 within the second compartment 366 without any interferences caused by the magnetic field within the capsule.

According to some embodiments, the at least one external magnet 340 comprise a permanent magnet, such as, but not limited to, ceramic, ferrite or Alnico magnets. In further embodiments, the at least one external magnet 340 is a permanent magnet.

According to some embodiments, the at least one external magnet 340 is configured to control the magnetic field direction within the capsule. In further embodiments, a change in the direction of the magnetic field induced by the at least one external magnet 340 is configured to control the magnetic field direction within the capsule.

In some embodiments, a change in the direction of the magnetic field induced by the at least one external magnet 340 comprises rotating said permanent magnet by 180°.

In some embodiments, the at least one external magnet 340 comprise an electromagnet. In some embodiments, the at least one external magnet 340 is an electromagnet. In some embodiments, the electromagnet is configured to modulate the external magnetic field. In further embodiments, a change in direction of the magnetic field induced by the at least one external magnet 340 comprise alternating the direction of the current flowing through said electromagnet. In still further embodiments, the electromagnet is configured to rotate the capsule 301 upon alternating the direction of the current flowing through said electromagnet.

According to the principles of the present invention, it is contemplated that a change in the direction of the magnetic field induced by an electromagnet will generate the same effects regarding the operation of capsule 301, as a change in the direction of the magnetic field induced by a permanent magnet.

In some embodiments, the electromagnet is an electropermanent magnet. The term “electropermanent magnet” or “EPM” as used herein, are interchangeable, and refers to a type of an electromagnet consisting of wire winding around a part of a permanent magnet. Exemplary embodiments regarding the operation of such electropermanent magnet are disclosed at International Publication No. WO 2018/073726 to the inventors, the entire contents of which incorporated herein by reference.

According to some embodiments, the external magnet kit further comprises fastening means (not shown) attached to the at least one external magnet 340, and configured to attach the at least one external magnet 340 to the body of a user. According to some embodiments, at least one external magnet 340 is positioned at a section along the fastening means, so that said section is located at a certain distance from the desired location in the GI tract in which the pharmaceutical composition is to be released. According to some embodiments, said certain distance is sufficient for the at least one external magnet 340 to successfully control the magnetic field direction within the capsule. According to some embodiments, said section is located at a minimal distance from the desired location in the GI tract in which the pharmaceutical composition is to be released. According to some embodiments, said section is located along the fastening means within a 50 cm radius, preferably within a 25 cm radius, preferably within a 10 cm radius, or more preferably within a 5 cm radius from the desired location of the GI tract in which the pharmaceutical composition is to be released.

The term “fastening means”, as used herein, refers to any elongated flexible structure known in the art, capable of being wrapped and unwrapped around the body of a user, such as a strap, a belt, a cord, a cable, a chain and the like.

In some embodiments, the external magnet kit further comprises a control system 380 (see FIG. 4) configured to change the magnetic field direction of the at least one external magnet 340, thereby controlling the release of the pharmaceutical composition from the capsule 301 to the GI tract, optionally by actuating the rotation thereof, wherein the control system 380 is in operative communication with the at least one external magnet 340. In further embodiments, said control system 380 is an automatic control system.

The control system 380 can be attached directly to the fastening means and/or to the at least one external magnet 340. Alternately, the control system 380 can be separated from the fastening means and/or the at least one external magnet 340, and further comprise at least one communication module configured to transfer commands and/or data thereto (utilizing wired and/or wireless communication methods and/or devices).

In some embodiments, the control system 380 comprises a processor and/or a mechanical driver. In further embodiments, the mechanical driver (e.g., an actuator) is configured to control the release of at least one pharmaceutical active agent from the capsule 301 by inducing a change in the direction of the magnetic field generated by the at least one external magnet, optionally by actuating the rotation thereof. In further embodiments, the processor can be selected from, but not limited to, a microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations, other manipulations of information, or any other function required therefrom. Each possibility represents a separate embodiment. The term “processor”, as used herein, refers to a single chip device which includes a plurality of modules which may be collected onto a single chip in order to perform various computer-related functions.

The present inventors have developed advanced capsules and drug delivery systems for magnetically controlled drug release, configured to enable release of a measured or a predetermined volume of a pharmaceutical composition, at a desired location of the GI tract. Said capsules comprise an advantageous magnetic mechanism, configured to enable controlled drug release of a predetermined volume of pharmaceutical active agents, as a function of changes in the magnetic field induced by the at least one external magnet. Advantageously, said mechanism can enable a user to directly control the drug release mechanism and optionally the expedite the process of the drug release from the capsule.

According to some embodiments, the first stationary magnet 320 and the second stationary magnet 321 are configured to direct the capsule 301 to a desired location of the GI tract, in response to the intensity of the magnetic field induced by the at least one external magnet.

According to some embodiments, the first stationary magnet 320 and the second stationary magnet 321 are configured to capture and to secure the capsule 301 to a desired location of the GI tract, in response to the intensity of the magnetic field, induced by the at least one external magnet. In further embodiments, the at least one external magnet is configured to capture and to secure the capsule 301, at a desired location of the GI tract.

Without wishing to being bound by any theory or mechanism of action, it is contemplated that when the south magnetic pole of external magnet 340 is facing the outer layer of the skin 350 of a user, it magnetically attracts the north magnetic poles of the first stationary magnet 320 and the second stationary magnet 321. As a result, the north magnetic poles of the first stationary magnet 320 and the second stationary magnet 321 orient themselves in the direction of the south magnetic pole of external magnet 340, and secure capsule 301 at a desired location of the GI tract.

Once capsule 301 arrives at the desired location of the GI tract, the first stationary magnet 320 and the second stationary magnet 321 are configured to secure the first surface 302 of capsule 301 to GI wall 352, as shown at FIG. 4. Fluid from the interior of the GI tract 353 can flow through the first opening 330 into the first compartment 364. Since the first compartment 364 is in fluid communication with the second compartment 366, as was described herein above, the fluid from the interior of the GI tract 353 can flow from the first compartment 364 into the second compartment 366. In some embodiments, the first compartment 364 and the second compartment 366 comprise fluid from the interior of the GI tract 353, following the arrival of capsule 301 at the desired location of the GI tract. In some embodiments, the at least one mobile member 373 is configured to be magnetized, and is magnetically attracted to the at least one external magnet 340. In further embodiments, the mobile member 373 is configured to move along the second compartment 366 in the direction of the external magnet 340, until said mobile member 373 is adjacent to the first surface 302 of capsule 301.

In some embodiments, the first stationary magnet 320 and the second stationary magnet 321 are configured to rotate the capsule around a central longitudinal axis of the capsule 301, in response to change in the magnetic field direction within the capsule induced by the at least one external magnet 340.

Without further wishing to being bound by any theory or mechanism of action, it is contemplated that when the external magnet 340 is rotated so that the north magnetic pole of external magnet 340 is facing the outer layer of the skin 350 of a user, it magnetically attracts the south magnetic pole of the first stationary magnet 320 and the second stationary magnet 321. As a result, the first stationary magnet 320 and the second stationary magnet 321 rotates around their horizontal axis in order to direct their south magnetic poles in the direction of external magnet 340. In some embodiments, the first stationary magnet 320 and the second stationary magnet 321 perform a 180° rotation around their horizontal axis. In some embodiments, the rotation of the first stationary magnet 320 and the second stationary magnet 321 is configured to rotate the capsule 301 along the central longitudinal axis of the capsule 301, and to secure the second surface 303 of capsule 301 to the GI wall 352. In some embodiments, the first stationary magnet 320, the second stationary magnet 321, or both, are configured to rotate the capsule 301 responsive to a change in the direction of the magnetic field of the at least one external magnet 340.

In some embodiments, the mobile member 373 is configured to be magnetized and to move along second compartment 366, in response to changes in direction of the external magnetic field induced by the at least one external magnet. In further embodiments, the movement of mobile member 373 along second compartment 366 is configured to pump fluid from the interior of the GI tract 353 into first compartment 364, and vice versa.

Without further wishing to being bound by any theory or mechanism of action, it is contemplated that the at least one mobile member 373 is configured to be magnetized, and is magnetically attracted to the at least one external magnet 340. Following the rotation of capsule 301, mobile member 373 is configured to move from the first surface 302 of capsule 301, along second compartment 366 in the direction of external magnet 340, until said mobile member 373 is adjacent to the second surface 303 of capsule 301. As mobile member 373 moves along the second compartment 366, it thrusts the fluid residing in front of it, and attracts the fluid residing behind it. The fluid that is thrusted or discharged in front of mobile member 373 flows from second compartment 366 in the direction of the first compartment 364, and from the first compartment 364 through the first opening 330 into the interior of the GI tract 353. The fluid that is attracted or sucked by mobile member 373 flows from the interior of the GI tract 353, through the second opening 331, into second compartment 366. Without wishing to be bound by any theory or mechanism of action, it is contemplated that second opening 331 is configured to equalize pressure along the second compartment 366 and/or to prevent low pressure from building between the two ends of mobile member 373. As described above, in some embodiments, mobile member 373 is dimensioned and shaped to extend from at least one partitioning element 371 to the wall 3601. Thus, mobile member 373 acts as a piston sliding along wall 3601 and at least one partitioning element 371.

An additional change in the direction of the magnetic field induced by the at least one external magnet 340, such as rotating the external magnet 340 so that the south magnetic pole of the external magnet 340 is facing against the outer layer of the skin 350 of a user, will cause the first stationary magnet 320 and the second stationary magnet 321 to rotate the capsule 301 again, so that the first surface 302 of capsule 301 is secured to the GI wall 352. Mobile member 373 will move within the second compartment 366 in the direction of external magnet 340, until said mobile member 373 is adjacent to the first surface 302 of capsule 301.

As mobile member 373 moves again within second compartment 366, it thrusts the fluid residing in from of it, and attracts the fluid residing behind it. The fluid that is thrusted or discharged in front of mobile member 373 flows from second compartment 366, through the second opening 331, into the interior of the GI tract 353. The fluid that is attracted or sucked by mobile member 373 flows from the interior of the GI tract 353, through the first opening 330, into first compartment 364, and from first compartment 364 in the direction of second compartment 366.

Each additional change in the direction of the external magnetic field induced by the at least one external magnet 340 causes the first stationary magnet 320 and the second stationary magnet 321 to rotate the capsule so that the first surface 302 and the second surface 303 of capsule 301 are alternately secured to the GI wall 352, as described herein above. Additionally, each additional change in the direction of the magnetic field induced by the at least one external magnet 340 cause the mobile member 373 to pump fluid in and out between the interior of the GI tract 353 and the first compartment 364.

When fluid from the interior of the GI tract 353 enters the first compartment 364 it starts to dissolve the pharmaceutical composition 310. In some embodiments, the first compartment 364 comprises fluid from the interior of the GI tract 353 and a fraction of the pharmaceutical composition and/or at least one pharmaceutical active agent, which upon the dissolution of pharmaceutical composition 310 are released by diffusion into the first compartment 364. As the capsule 301 continues to rotate and the mobile member 373 continues to pump fluid in and out between the interior of the GI tract 353 and the first compartment 364, a fraction of the pharmaceutical composition and/or pharmaceutical active agents from the pharmaceutical composition 310 are released from the first compartment 364 into the interior of the GI tract 353, and new fluid from the interior of the GI tract 353 is being pumped into the first compartment 364. Said new fluid from the interior of the GI tract 353 continues to dissolve the pharmaceutical composition 310, and therefore the pharmaceutical composition and/or at least one pharmaceutical active agent from the pharmaceutical composition 310 continue to being discharged from the first compartment 364 into the interior of the GI tract 353.

In some embodiments, the mobile member 373 is configured to pump fluid from the first compartment 364, said fluid comprising the pharmaceutical composition and/or at least one pharmaceutical active agent from the pharmaceutical composition 310, into the interior of the GI tract 353, and vice versa, in response to changes in direction of the external magnetic field, induced by the at least one external magnet 340. In further embodiments, the mobile member 373 is configured to pump new fluid from the interior of the GI tract 353 into the first compartment 364, for continued release of the pharmaceutical composition and/or at least one pharmaceutical active agent from the pharmaceutical composition 310.

In some embodiments, the volumes of the fluid that are being pumped in and out between the interior of the GI tract 353 and the first compartment 364 are identical, and are determined by the free available volume of the second compartment 366, i.e. the volume of the second compartment 366 minus the volume of the mobile member 373, or the inner space of the second compartment 366 without the space/volume of the mobile member 373, meaning the space/volume of the second compartment 366 which is free to hold/contain fluid.

In some embodiments, the free available volume is in the range of about 0.001 ml to about 1 ml. In further embodiments, the free available volume is in the range of about 0.01 ml to about 0.1 ml. In still further embodiments, the free available volume is in the range of about 0.01 ml to about 0.05 ml.

Advantageously, the free available volume is predetermined upon the manufacturing of capsule 301, and therefore the volume of the pharmaceutical composition or the dosage that is being released from the first compartment 364 upon each rotation is predetermined. The term “dosage”, as used herein, refers to the volume of the pharmaceutical composition and/or the at least one pharmaceutical active agent from the pharmaceutical composition 310 that are being discharged into the interior of the GI tract 353 from the first compartment 364 upon each rotation of the capsule 301.

In some embodiments, advantageously, the movement of the mobile member 373 as presented herein above is configured to enable the release of a predetermined dosage of the pharmaceutical composition and/or at least one pharmaceutical active agent from the capsule 301, and optionally to expedite the release thereof from the capsule into the interior of the GI tract 353, thereby enabling an improved drug release mechanism.

In some embodiments, the mobile member 373 is configured to enable release of a predetermined volume of the pharmaceutical composition and/or the at least one pharmaceutical active agent from capsule 301, in response to changes in the direction of the magnetic field induced by the at least one external magnet 340. In further embodiments, the at least one external magnet 340 is configured to control the flow of the pharmaceutical composition and/or the at least one pharmaceutical active agent from the capsule 301, as a function of changes in the magnetic field induced by the at least one external magnet 340. Therefore, the flow of the pharmaceutical composition through the opening first opening 330 is controlled responsive to the direction of the magnetic field of the at least one external magnet 340. In still further embodiments, the at least one external magnet 340 is configured to control the magnetic field direction within the capsule generated by the at least one stationary magnet 320, thereby controlling the flow of the pharmaceutical active agents from the capsule to the GI tract.

According to some embodiments, as shown in FIG. 3H, capsule 301 further comprise a permeable membrane 368 and a pharmaceutically acceptable carrier fluid 307. As was described herein above, changes in direction of the magnetic field induced by the at least one external magnet 340 cause the mobile member 373 to pump fluid in and out between the interior of the GI tract 353 and the first compartment 364. At the first rotation of capsule 301, a fraction of the pharmaceutically acceptable fluid will be discharged from the capsule 301, and a fraction of fluid from the interior of the GI tract 353 will enter the capsule 301. After several rotations of the capsule 301, the fluid residing inside the first compartment 364 will comprise pharmaceutically acceptable carrier fluid 307, fluid from the interior of the GI tract 353, and the pharmaceutical composition and/or the at least one pharmaceutical active agent from pharmaceutical composition 310. In some embodiments, the pharmaceutically acceptable carrier fluid 307 does not hinder or affect the release of the pharmaceutical composition 310 and/or the at least one pharmaceutical active agent from the first compartment 364.

In some embodiments, capsule 301 is configured to be removed from the GI tract by changes in the magnetic field induced by the at least one external magnet 340. Said changes could comprise performing at least one action, such as weakening or shutting down the magnetic field, or removing the at least one external magnet 340 from the skin of a user 350. Following the execution of such action, the capsule 301 will no longer be secured to the GI wall 352 and can be carried out of the body through the GI tract, via peristaltic movement of the GI tract and/or by fluid flow along the GI tract. The term “peristaltic movement” as used herein, refers to the radially symmetrical contraction and relaxation of muscles in the form of a wave which propels partially digested food materials and fluids down the gastrointestinal tract.

According to some embodiments, there is provided a method of treating a disease or disorder in a subject in need thereof, the method comprises:
providing system 300, wherein the pharmaceutical composition 310 comprises at least one pharmaceutically active agent for treating the disease or disorder;
attaching at least one external magnet 340 to the skin of the user; and
administering, per os, capsule 301.

In some embodiments, the method comprises attaching the external magnet 340 to the skin of a user, at the vicinity of a desired location of the GI tract, in which the pharmaceutical composition 310 is to be released. In further embodiments, the method further comprises directing the capsule 301 to the desired location of the GI tract utilizing the magnetic field induced by the at least one external magnet 340. In still further embodiments, the method further comprises rotating the capsule 301 and releasing the pharmaceutical composition at the desired location of the GI tract, in response to changes in direction of the magnetic field induced by the at least one external magnet 340.

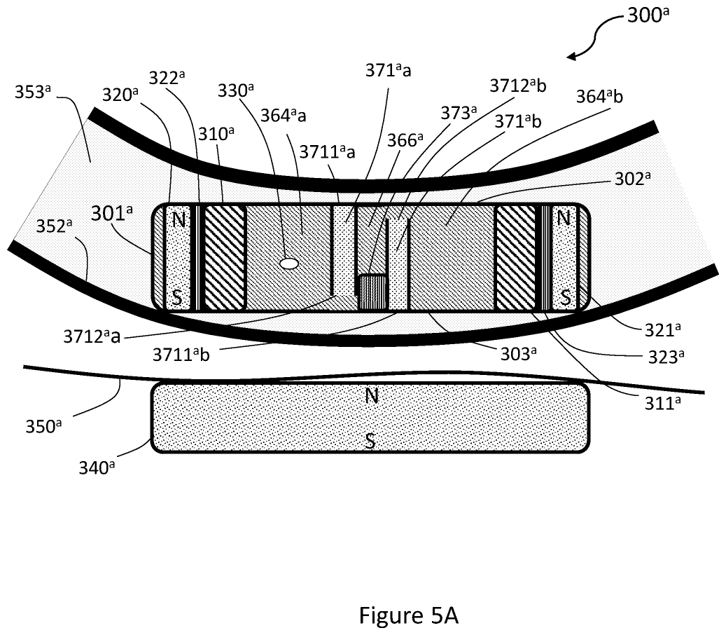

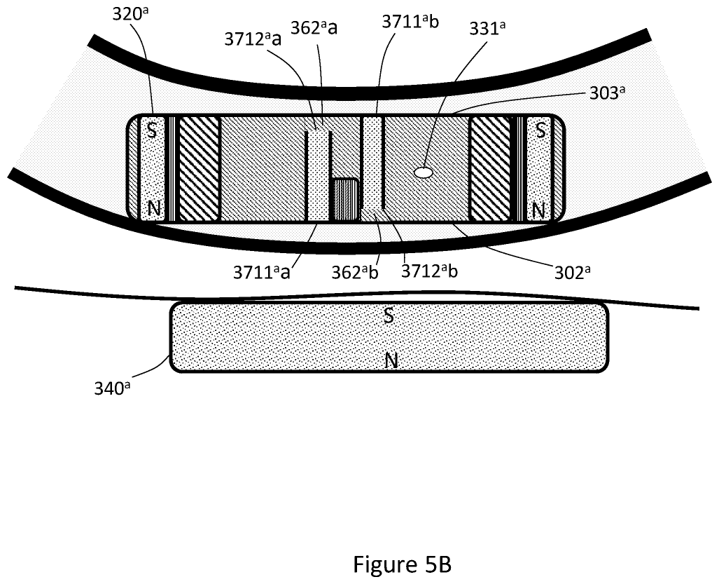

Reference is now made to FIGS. 5A-5B. FIGS. 5A-5B constitute a cross-sectional view of drug delivery system 300^{a}, depicting an exemplary embodiment of drug delivery system 300. Unless stated otherwise for specific components or configurations, the drug delivery system 300^{a} is of the same construction, configuration and operation as that of drug delivery system 300 shown in FIG. 4, with like numbers referring to like parts, except that: capsule 301^{a} comprises a plurality of portioning elements, dividing the drug chamber into a corresponding plurality of compartments, one compartment comprises the mobile member 373^{a} and each one of the remaining compartments comprises at least one pharmaceutical composition 310^{a}, wherein each one of said remaining compartments is in fluid communication with the one compartment comprising the mobile member 373^{a} and wherein each one of said remaining compartments comprises an opening.

According to some embodiments, the drug chamber 360^{a} comprise a first partitioning element 371^{a}a and a second partitioning element 371^{a}b, wherein said first partitioning element 371^{a}a and second partitioning element 371^{a}b partitions the drug chamber 360^{a} to the following compartments: a first compartment 364^{a}a extending between one side of the first partitioning element 371^{a}a and the first stationary magnet 320^{a}; a second compartment 366^{a} extending between the other side of the first partitioning element 371^{a}a and one side of the second partitioning element 371^{a}b; and a third compartment 364^{a}b extending between the other side of the second partitioning element 371^{a}b and the second stationary magnet 321^{a} or alternately an edge of the capsule 301^{a}.

According to some embodiments, the first partitioning element 371^{a}a is oriented perpendicularly to the central longitudinal axis of capsule 301^{a} and comprises a first partitioning element end 3711^{a}a and a second partitioning element end 3712^{a}a, such that the first partitioning element end 3711^{a}a is attached to the first surface 302^{a} of capsule 301^{a} and the second partitioning element end 3712^{a}a is spaced from the second surface 303^{a} of capsule 301^{a}, thereby forming a first passage 362^{a}a between the second partitioning element end 3712^{a}a and the second surface 303^{a}. In further embodiments, the first passage 362^{a}a is configured to provide a fluid communication between said first compartment 364^{a}a and the second compartment 366^{a}.

According to some embodiments, the second partitioning element 371^{a}b is oriented perpendicularly to the central longitudinal axis of capsule 301^{a} and comprises a first partitioning element end 3711^{a}b and a second partitioning element end 3712^{a}b, such that the first partitioning element end 3711^{a}b is attached to the second surface 303^{a} of capsule 301^{a} and the second partitioning element end 3712^{a}b is spaced from the first surface 302^{a} of capsule 301^{a}, thereby forming a second passage 362^{a}b between the second partitioning element end 3712^{a}b and the first surface 302^{a} of capsule 301^{a}. In further embodiments, the second passage 362^{a}b is configured to provide a fluid communication between said second compartment 366^{a} and the third compartment 364^{a}b.

In some embodiments, the first passage 362^{a}a and the second passage 362^{a}b are located on parallelly opposite surfaces of the capsule 301^{a}.

According to some embodiments, the first compartment 364^{a}a comprise the first opening 330^{a}, wherein the first opening 330^{a} is located on or embedded within, the surface of the first compartment 364^{a}a. In some embodiments, the second compartment 366^{a} comprises the mobile member 373^{a} disposed therein, wherein said mobile member 373^{a} is configured to be magnetized and to move within the second compartment 366^{a}, perpendicularly to the longitudinal axis, between the first surface 302^{a} and the second surface 303^{a} of capsule 301^{a}. In further such embodiments, the mobile member 373^{a} is configured to be magnetized and to move within the second compartment 366^{a} in response to changes in direction of the external magnetic field induced by the at least one external magnet 340^{a}. In still further such embodiments, the mobile member 373^{a} is configured to alternately move through the second compartment 366^{a} towards the fluid connection to the third compartment 364^{a}b (i.e., the second passage 362^{a}b) and away therefrom. In some embodiments, the third compartment 364^{a}b comprise the second opening 331^{a}, wherein the second opening 331^{a} is located on or embedded within, the surface of the third compartment 364^{a}b.

The first opening 330^{a} can be positioned in a variety of locations along the surface of the first compartment 364^{a}a, while the second opening 331^{a} can be positioned in a variety of locations along the surface of the third compartment 364^{a}b. According to some embodiments, the first opening 330^{a} and the second opening 331^{a} are located on/at parallelly opposite surfaces of the capsule 301^{a}, as illustrated at FIGS. 5A and 5B, optionally for improved release of the pharmaceutical composition from the capsule into the GI tract. However, it is to be understood that the first opening 330^{a} and the second opening 331^{a} can be located at any parallel surfaces of the first compartment 364^{a}a and the third compartment 364^{a}b, respectively, including surfaces facing the same direction (i.e., not opposite surfaces).

According to some embodiments, the first stationary magnet 320^{a} and the second stationary magnet 321^{a} are configured to direct (or to capture and secure) the capsule 301^{a} to a desired location of the GI tract, in response to the intensity of the magnetic field induced by the at least one external magnet 340^{a}. Without wishing to being bound by any theory or mechanism of action, it is contemplated that when the north magnetic pole of the external magnet 340^{a} is facing the outer layer of the skin 350^{a} of a user, it magnetically attracts the south magnetic poles of the first stationary magnet 320^{a} and the second stationary magnet 321^{a}. As a result, the south magnetic poles of the first stationary magnet 320^{a} and the second stationary magnet 321^{a} orient themselves in the direction of the north magnetic pole of external magnet 340^{a}, and secure capsule 301^{a} at a desired location of the GI tract.

Once the capsule 301^{a} arrives at the desired location of the GI tract, the first stationary magnet 320^{a} and the second stationary magnet 321^{a} are configured to secure the second surface 303^{a} of capsule 301^{a} to GI wall 352^{a}, as shown at FIG. 5A.

Fluid from the interior of the GI tract 353^{a} can flow through the first opening 330^{a} and the second opening 331^{a}, and into the first compartment 364^{a}a and the third compartment 364^{a}b, respectively. The movable component 373^{a} is configured to be magnetized, and is magnetically attracted to the at least one external magnet 340^{a}. In some embodiments, the movable component 373^{a} is configured to move along second compartment 366^{a} in the direction of external magnet 340^{a}, until said movable component 373^{a} reaches the second surface 303^{a} of the capsule 301^{a}.

In some embodiments, the first stationary magnet 320^{a} and the second stationary magnet 321^{a} are configured to rotate the capsule 301^{a} around a central longitudinal axis of the capsule 301^{a}, in response to a change in direction of the magnetic field induced by the at least one external magnet 340^{a}. In further embodiments, the at least one external magnet 340^{a} is configured to control the magnetic field direction within the capsule 301^{a}.

Following the rotation of capsule 301^{a}, mobile member 373^{a} is configured to move from the second surface 303^{a} of capsule 301^{a}, along second compartment 366^{a} in the direction of external magnet 340^{a}, until said mobile member 373^{a} is adjacent to the first surface 302^{a} of capsule 301^{a}. As mobile member 373^{a} moves along second compartment 366^{a}, it thrusts the fluid residing in from of it, and attracts the fluid residing behind it. The fluid that is thrusted or discharged in front of mobile member 373^{a} flows from the second compartment 366^{a} in the direction of the third compartment 364^{a}b through the second passage 362^{a}b, and from the third compartment 364^{a}b through the second opening 331^{a} into the interior of the GI tract 353^{a}. The fluid that is attracted or sucked by mobile member 373^{a} flows from the interior of the GI tract 353^{a}, through the first opening 330^{a}, into the first compartment 364^{a}a.

An additional change in the direction of the magnetic field induced by the at least one external magnet 340^{a}, such as rotating the external magnet 340^{a}, will result in the rotation of the capsule 301^{a} so that the second surface 303^{a} of capsule 301^{a} is secured to the GI wall 352^{a}, and the mobile member 373^{a} will move within the second compartment 366^{a} in the direction of external magnet 340^{a}, until said mobile member 373^{a} is adjacent to the second surface 303^{a} of capsule 301^{a}. As mobile member 373^{a} moves again within second compartment 366^{a}, it thrusts the fluid residing in from of it, and attracts the fluid residing behind it. The fluid that is thrusted or discharged in front of mobile member 373^{a} flows from the second compartment 366^{a} in the direction of the first compartment 364^{a}a through the first passage 362^{a}a, and from the first compartment 364^{a}a through the first opening 330^{a} into the interior of the GI tract 353^{a}. The fluid that is attracted or sucked by mobile member 373^{a} flows from the interior of the GI tract 353^{a}, through the second opening 331^{a}, into the third compartment 364^{a}b.

Each additional change in direction of the external magnetic field, induced by the at least one external magnet 340^{a}, will cause the mobile member 373^{a} to pump fluid from the interior of the GI tract 353^{a} into the first compartment 364^{a}a, and from the third compartment 364^{a}b into the interior of the GI tract 353^{a}, and vice versa. In some embodiments, the mobile member 373^{a} is configured to pump fluid from the interior of the GI tract 353^{a} into the first compartment 364^{a}a and the third compartment 364^{a}b, alternately, in response to a change in direction of the external magnetic field, induced by the at least one external magnet 340^{a}.

When fluid from the interior of the GI tract 353^{a} alternately enters the first compartment 364^{a}a and the third compartment 364^{a}b, it starts to dissolve the first pharmaceutical composition 310^{a} and the second pharmaceutical composition 311^{a}, accordingly. In some embodiments, the first compartment 364^{a}a comprise fluid from the interior of the GI tract 353^{a} and a fraction of the pharmaceutical composition and/or the at least one pharmaceutical active agent which upon the dissolution of the first pharmaceutical composition 310^{a} are released by diffusion into the first compartment 364^{a}a. In some embodiments, the third compartment 364^{a}b comprise fluid from the interior of the GI tract 353^{a} and a fraction of the pharmaceutical composition and/or the at least one pharmaceutical active agent, which upon the dissolution of the second pharmaceutical composition 311^{a} are released by diffusion into the third compartment 364^{a}b.

The mechanism in which the pharmaceutical composition and/or the at least one pharmaceutical active agent are released from the first compartment 364^{a}a and the third compartment 364^{a}b is similar to the mechanism in which the pharmaceutical composition and/or the at least one pharmaceutical active agent are released from the first compartment 364, as was described herein above, in accordance with some embodiments regarding capsule 301.

In some embodiments, the mobile member 373^{a} is configured to release the pharmaceutical composition and/or the at least one pharmaceutical active agent from the first compartment 364^{a}a and the third compartment 364^{a}b, alternately, in response to changes in the direction of the magnetic field induced by the at least one external magnet 340^{a}. In further embodiments, the mobile member 373^{a} is configured to release a predetermined volume of the pharmaceutical composition and/or the at least one pharmaceutical active agent from the first compartment 364^{a}a and the third compartment 364^{a}b, alternately, in response to changes in direction of the external magnetic field, induced by the at least one external magnet 340^{a}.

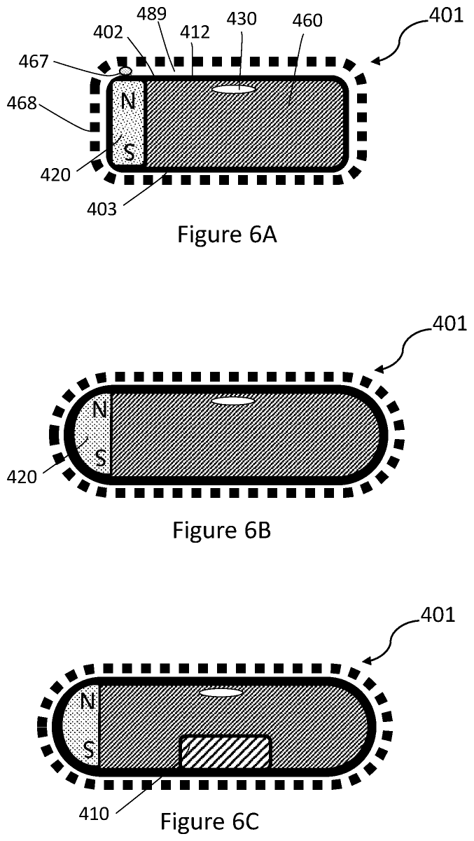

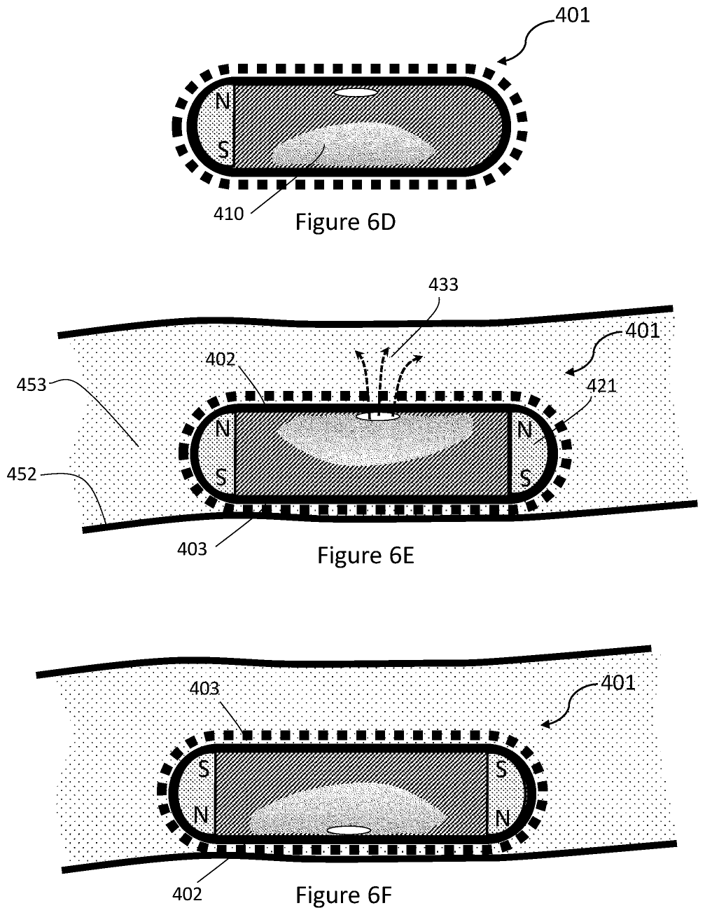

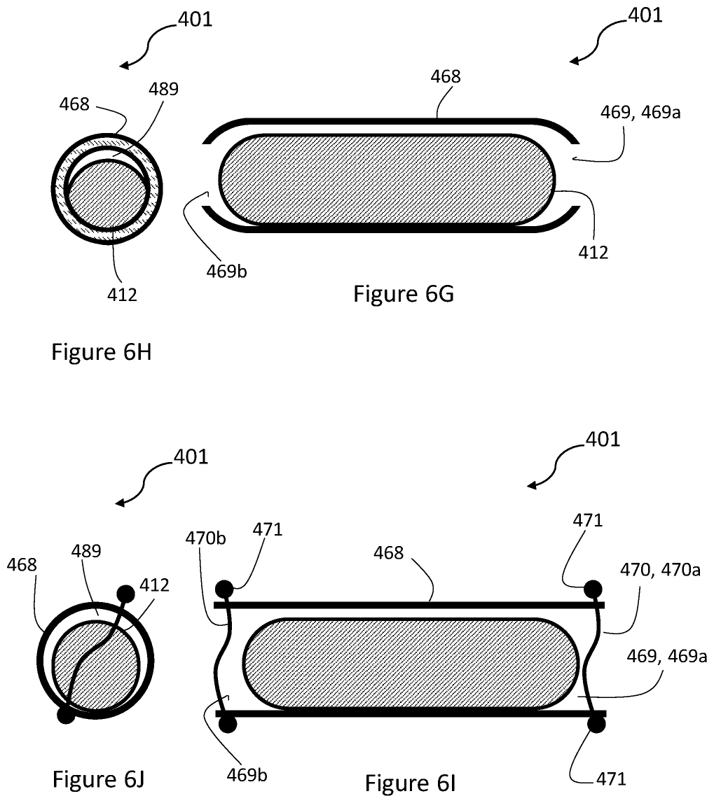

According to yet another aspect, there is provided a capsule 401. Reference is now made to FIGS. 6A-6G and 6I which constitute cross-sectional views depicting capsule 401 through different embodiments of the present invention. FIGS. 6H and 6J constitute as side cross-sectional views of FIGS. 6G and 6I, respectively.

According to some embodiments, capsule 401 comprises at least one stationary magnet 420; at least one drug chamber 460, wherein the at least one drug chamber 460 comprises or exhibits at least one opening 430; and a casing 468 encompassing at least the drug chamber 460 and preferably the entire capsule 401.

According to some embodiments, capsule 401 further comprises an external housing 412 configured to encompass the capsule 401. In further embodiments, said external housing 412 is covered by said casing 468. In still further embodiments, the casing 468 is configured to encompass the entire external housing 412.

According to some embodiments, casing 468 comprise a perforated material, configured to allow the pharmaceutical active agents to be released from capsule 401 via the at least one opening 430 into the interior of the GI tract. In further embodiments, the casing 468 comprise an elastic and/or soft material. In still further embodiments, the casing 468 is elastic.

In some embodiments, the geometrical shape and dimensions of casing 468 are configured to interact with the geometrical shape and dimensions of external housing 412, wherein the casing 468 is shaped to envelope or wrap the external housing 412.

According to some embodiments, casing 468 is directly attached to the external housing 412 of the capsule 401.

According to some embodiments, casing 468 is not directly attached to the external housing 412, yet it may occasionally contact any external surface of the external housing 412. According to some embodiments, the capsule 401 further comprise at least one spacing element 467 (see FIG. 6A), having a first spacing element end and a second spacing element end, wherein the first spacing element end is in contact with the external housing 412 and the second spacing element end is in contact with the casing 468. In further embodiments, said spacing element 467 is configured to space the casing 468 from the external housing 412 of the capsule 401, thereby forming a predetermined space 489 around the capsule 401. In further embodiments, the predetermined space 489 is defined between the casing 468 and the capsule 401 or the drug chamber 460 thereof. The at least one spacing element 467 can be positioned in a variety of locations along the circumference of external housing 412 of the capsule 401. In further embodiments, the capsule 401 comprise a plurality of spacing elements 467. In further embodiments, the predetermined space 489 is defined by the at least one spacing element 467.

In some embodiments, casing 468 is further configured to prevent from undesired substances residing in the interior of the GI tract to ender capsule 401. Said undesired substances can include solid particles originating from partially digested food substances.

In some embodiments, casing 468 is spaced from the external housing 412 via at least one spacing element 467. Advantageously, this arrangement enables the pharmaceutical composition and/or the pharmaceutical active agents to be released from capsule 401 through the at least one opening 430 on to space 489, and from space 489, through a variety of locations along the surface of casing 468 into the interior of the GI tract, thus providing a simple solution in case of clogging of said casing 468 at a specific location along its surface. For example, if solid particles originating from partially digested food substances were to clog the area along the surface of casing 468 which is parallel to the at least one opening 430, the space 489 between casing 468 and the external housing 412 will enable the release of the pharmaceutical composition and/or the pharmaceutical active agents from other locations along the surface of casing 468.

According to some embodiments, the perforated material comprises a durable and biocompatible material, configured to pass through the GI tract without dissolving or degrading in an undesired location. According to some embodiments, said durable and biocompatible material is selected from a biocompatible polymer, such as but not limited to: polyethylene glycol (PEG), polylactic acid (PLA), polymethylmethacrylate, polyethylmethacrylate, polybutylmethacrylate, poly-2-ethylhexylmethacrylate, polylaurylmethacrylate, polyhydroxylethyl methacrylate, poly-methylacrylate, 2-methacryloyloxyethylphosphorylcholine (MPC), polystyrene, poly n-ethyl-4-vinyl-pyridinium bromide, polyvinyl acetate, and derivatives and/or combinations thereof. Each possibility represents a separate embodiment of the present invention. In some embodiments, the perforated material comprises an enteric coating.

According to some embodiments, drug chamber 460 is configured to contain therewithin a pharmaceutical composition. According to some embodiments, the drug chamber 460 further comprises at least one pharmaceutical composition 410 comprising least one pharmaceutical active agent. According to further embodiments, the drug chamber 460 further comprises at least one pharmaceutical unit comprising the pharmaceutical composition 410.

According to some embodiments, the drug chamber 460 further comprises a plurality of pharmaceutical compositions 410. It is noted that the same componential and structural principles disclosed for pharmaceutical composition 310 similarly apply to pharmaceutical composition 410.

It is noted that the same functional and structural principles disclosed for the first opening 330 similarly apply to the at least one opening 430 which is located at the surface of the drug chamber 460, as presented at FIG. 6A.

Capsule 401 is manufactured to have a size and a shape appropriate for ingestion and for easy delivery through the various segments of the GI tract. It is noted that the same functional and structural principles disclosed for the size and the shape of capsule 301 similarly apply to capsule 401.

The drug chamber 460 may comprise a plurality of compartments, wherein at least one compartment is a drug compartment adapted to contain a pharmaceutical composition. It is to be understood that the drug compartment 460 may be filled with a pharmaceutical composition at the time of administration, prior to administration, and when being consumed by a subject but prior to its release from the capsule 401 at the desired location in the GI tract.

According to some embodiments, capsule 401 comprises an external housing 412 having the first surface 402 and the second surface 403 parallelly facing each other. It is noted that the same functional and structural principles disclosed for external housing 312 similarly apply to external housing 412.

In some embodiments, the stationary magnet 420 is disposed within an inner surface of the capsule 401, as shown at FIG. 6A. In further embodiments, the stationary magnet 420 is in contact with, or attached to, the shortest face of the inner surface of the capsule 401, perpendicularly to the central longitudinal axis of capsule 401. In further embodiments, the at least one stationary magnet 420 is configured to generate/output a magnetic field external to the capsule 401. It is noted that the same functional and structural principles disclosed for stationary magnet 320 similarly apply to stationary magnet 420.

FIG. 6B illustrates yet another embodiment of the capsule 401 which differs from the embodiments of FIG. 6A in that the cross-sectional shape of the at least one stationary magnet 420 is dome-shaped, wherein the curvilinear section of stationary magnet 420 is extending towards the surface edge of the capsule 401 in the shortest dimension thereof, and the flat section of stationary magnet 420 is extending towards drug chamber 460. It is to be understood, however, that the cross-sectional geometry of stationary magnet 420 is optionally different, such as a circular, triangular or any other curvilinear or rectilinear cross-section. Each possibility represents a separate embodiment of the present invention. In further embodiments, the geometrical shape of casing 468 is similar to the geometrical shape of external housing 412, as illustrated at FIG. 6B.

FIG. 6C illustrates yet another embodiment of the capsule 401 which differs from the embodiments of FIG. 6B in that capsule 401 further comprise at least one pharmaceutical composition 410, wherein said pharmaceutical composition 410 is disposed within the drug chamber 460.

In some embodiments, the pharmaceutical composition 410 is encapsulating, or otherwise containing, a pharmaceutical composition therein. In some embodiments, the pharmaceutical composition 410 is formulated as solid dosage form of a pharmaceutical composition, such as, pill, tablet, granules and globules among others.

According to some embodiments, the at least one pharmaceutical composition 410 comprises a pharmaceutical composition comprising at least one pharmaceutical active agent, as was presented herein above.

FIG. 6D illustrates yet another embodiment of the capsule 401 which differs from the embodiments of FIG. 6C in that the pharmaceutical composition 410 is formulated as semi-solid dosage form, such as a gel.

FIG. 6E illustrates an exemplary embodiment of the capsule 401 which differs from the embodiments of FIG. 6D in that the at least one stationary magnet 420 comprise a first stationary magnet 420 and a second stationary magnet 421.

In some embodiments, the second stationary magnet 421 is disposed within an inner surface of the capsule 401, parallelly facing the first stationary magnet 420, as shown at FIG. 6E. In further embodiments, the second stationary magnet 421 is in contact with, or attached to, the shortest face of the inner surface of the capsule 401, perpendicularly to the central longitudinal axis of capsule 401, parallelly facing the first stationary magnet 420.

According to some embodiments, the first stationary magnet 420 and the second stationary magnet 421 are configured to direct (or to capture and secure) the capsule 401 to a desired location of the GI tract, as a function of the intensity of a magnetic field induced by at least one external magnet. According to some embodiments, the first stationary magnet 420 and the second stationary magnet 421 are configured to secure the second surface 403 of capsule 401 to GI wall 452, at a desired location in the GI tract, as shown at FIG. 6E. In further embodiments, the drug chamber 460 is configured to release the pharmaceutical composition and/or the pharmaceutical active agents through the at least one opening 430, on towards casing 468 through space 489, and finally into the interior of the GI tract 453. Schematic depiction of flow lines 433 depict the direction in which the pharmaceutical composition flow from drug chamber 460 into the interior of the GI tract 453, as illustrated at FIG. 6E. In some embodiments, the pharmaceutical composition and/or the pharmaceutical active agents can be released out from the drug chamber 460 by diffusion, by a gravitational force, by erosion, and combinations thereof. Each possibility represents a separate embodiment of the present invention.

In some embodiments, the first stationary magnet 420 and the second stationary magnet 421 are configured to rotate the capsule 401 along its central longitudinal axis, along the GI wall 452, in response to changes in the direction of a magnetic field induced by at least one external magnet, as described above. The rotation of the capsule is configured to secure the first surface 402 of capsule 401 to GI wall 452, so that the at least one opening 430 is sealed by the GI wall 452 through casing 468, in a manner which temporarily eliminates/reduces the space 489, due to the elasticity of the casing 468, and therefore the release of pharmaceutical active agents from drug chamber 460 is prevented, as illustrated at FIG. 6F. In some embodiments, as the capsule 401 is pulled against GI wall 452 by the at least one external magnet, the force applied therebetween compresses the casing 468 towards the capsule 401 such that the casing 468 covers the at least one opening 430 such that the pharmaceutical active agents aren't released therethrough.

In some embodiments, the capsule 401 is configured to transition between an open state and a closed state, wherein in the closed state the capsule 401 is secured to the GI wall 452 so that the at least one opening 430 is sealed thereby, through casing 468, thus preventing the pharmaceutical composition and/or the pharmaceutical active agents to exit the capsule, and wherein in the open state the capsule 401 is secured to the GI wall 452 so that the pharmaceutical composition and/or the pharmaceutical active agents are released through the at least one opening 430, through casing 468, towards the interior of the GI tract 453. In further embodiments, in the closed state of the capsule 401 the at least one opening 430 faces the GI wall 452 and is sealed thereby, through casing 468. In further embodiments, in the open state of the capsule 401 the at least one opening 430 does not faces the GI wall 452 and is facing the interior of the GI tract 453. In still further embodiments, in the open state of the capsule 401, preferably, the opening 430 is located on a surface of the capsule which is oppositely parallel to the surface which is secured against the GI wall 452.

The rotation of capsule 401 along its longitudinal axis along the GI wall 452 can graze or damage the surface of the inner GI wall, due to the relatively large contact area between the GI wall 452 and the capsule 401, resulting from the geometrical external shape of the external housing 412 of capsule 401.

Advantageously, the inventors developed the casing 468, adapted to provide protection for the surface of the GI wall from capsule 401, by spacing between the external housing 412 of the capsule 401 and the GI wall 452. In some embodiments, casing 468 is configured to protect the surface of the inner GI wall from damage inflicted upon during the rotation of the capsule 401 along its central longitudinal axis, along the inner GI wall 452.

Although not treated in full detail, it should be readily understood that capsule 301 according to any of the configurations described herein above, can be used in combination with a casing 468 disposed around the external housing the capsule 301, according to any of the configurations described herein above.

FIGS. 6G and 6H illustrates yet another embodiment of the capsule 401 which differs from the embodiments of FIGS. 6A-6F in that the casing 468 comprises at least one casing opening 469. The at least one casing opening 469 can enable the insertion of the capsule 401 into the casing 468 for the utilization thereof, as described herein above. In further embodiments, the casing 468 comprises a first casing opening 469a and a second casing opening 469b, as illustrated at FIG. 6G.

In some embodiments, the casing 468 is configured to encompass at least a portion of the external housing 412.

In some embodiments, the capsule 401 is configured to be inserted into the casing 468 via the at least one casing opening 469. The at least one casing opening 469 can be shrunken/diminished after the insertion thereof, so that the diameter of the at least one casing opening 469 is small enough not to allow the capsule exit from the casing 468. The at least one casing opening 469 can be shrunken/diminished by the application of heat, or by any mechanical or physical crimping mechanism known in the art.

FIGS. 6I and 6J illustrates yet another embodiment of the capsule 401 which differs from the embodiments of FIGS. 6A-6F in that the casing 468 comprises at least one casing opening 469 and at least one elongated member 470 configured to seal at least a portion of said at least one casing opening 469. The at least one casing opening 469 can enable the insertion of the capsule 401 into the casing 468 for the utilization thereof, as described herein above. In further embodiments, the at least one elongated member 470 extends from opposite surfaces of the at least one casing opening 469, in order to reduce the diameter thereof and/or to provide at least a partial seal thereof, thereby preventing the capsule from exiting from within the casing 468.

In some embodiments, the casing 468 comprises a first casing opening 469a and a second casing opening 469b, wherein the first casing opening 469a is sealed by a first elongated member 470a, and wherein the second casing opening 469b is sealed by a second elongated member 470b, as illustrated at FIG. 6I. In further embodiments, the first elongated member 470a, the second elongated member 470b, or both, comprise a plurality of elongated members.

In some embodiments, the casing 468 has a curvilinear shape, corresponding to the shape of the capsule (not shown), or a tubular shape as illustrated at FIG. 6I.

In some embodiments, the at least one elongated member 470 comprises a biocompatible material, as presented herein above. In some embodiments, the at least one elongated member 470 is elastic and/or flexible, and is selected from a string, a chain, a wire, a fabric, combinations thereof, or any other suitable material.

In some embodiments, the at least one elongated member 470 is inserted through at least two apertures, each is located at opposite surfaces of the at least one casing opening 469, in order to secure it thereto. In further embodiments, the at least one elongated member 470 is inserted through a plurality of apertures, located at the circumference of the at least one casing opening 469. The at least one elongated member 470 can be sutured around the at least one casing opening 469, in various suture patterns known in the art.

In some embodiments, the at least one elongated member 470 comprises at least one sealing element 471 located at at least one edge thereof, adapted to secure the at least one elongated member 470 in its place and to prevent accidental departure/extraction thereof. The at least one sealing element 471 can be formed by at least one technique selected from: heating and melting the edge of the elongated member 470, forming a knot in the edge thereof, or by attaching a stopping object to the elongated member 470 in the edge thereof (e.g., a bid). In further embodiments, the at least one elongated member 470 comprises at least two sealing elements 471 located at opposite edges thereof, as illustrated at FIG. 6I.

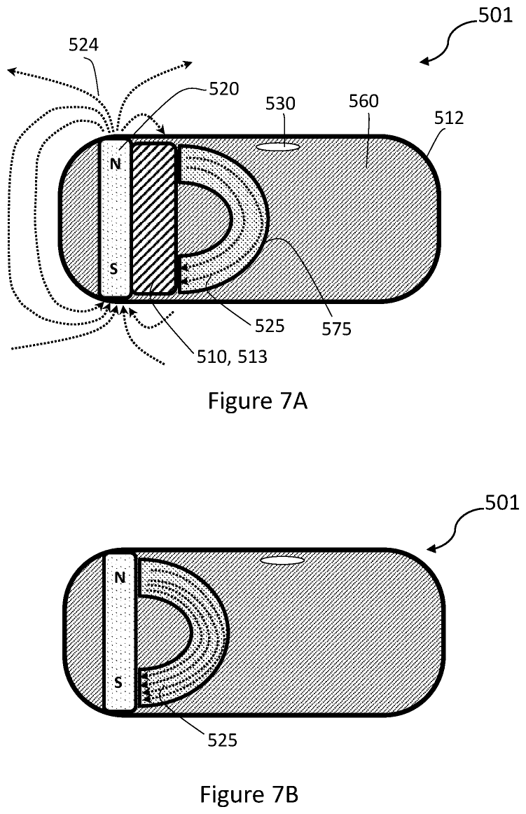

According to another aspect, there is provided a capsule 501. Reference is now made to FIGS. 7A-10B which constitutes a cross-sectional view depicting capsule 501 through different embodiments of the present invention.

According to some embodiments, capsule 501 comprises at least one stationary magnet 520; and at least one drug chamber 560, wherein the at least one drug chamber 560 comprises or exhibits at least one opening 530; at least one pharmaceutical composition 510 comprising at least one pharmaceutical active agent; and at least one magnetic shunt 575, wherein the at least one pharmaceutical composition 510 and the at least one magnetic shunt 575 are disposed within said drug chamber 560, and wherein prior to the release the pharmaceutical composition and/or the pharmaceutical active agents from capsule 501 at the desired location in the GI tract the at least one pharmaceutical composition 510 is positioned between the at least one stationary magnet 520 and the at least one magnetic shunt 575, thereby separating them from one another and preventing contact therebetween. Thus, the at least one pharmaceutical composition 510 constitutes as at least one buffer 513, such that the at least one buffer 513 separates the at least one stationary magnet 520 from the at least one magnetic shunt 575.

According to some embodiments, capsule 501 further comprises an external housing 512 configured to encompass the capsule 501.

The at least one stationary magnet 520 is configured to generate/output a magnetic field external to the capsule. In some embodiments, the at least one magnetic shunt 575 is configured to be magnetized and is attracted to the internal magnetic field induced by at least one stationary magnet 520.

First flux lines 524 illustrate a fraction of the magnetic flux generated by the at least one stationary magnet 520, that flow from the north pole to the south pole of the at least one stationary magnet 520, passing through the external housing 512 and various segments of drug chamber 560, excluding the at least one magnetic shunt 575. Second flux lines 525 illustrate the remaining fraction of the magnetic flux generated by the at least one stationary magnet 520, flow from the north pole to the south pole of the at least one stationary magnet 520, passing solely through the at least one magnetic shunt 575, as illustrated at FIG. 7A.

It is understood that the schematic depiction of magnetic flux lines in FIGS. 7A-9B are for purposes of explanation only and that varying and/or additional flux lines may be present.

The term “magnetic shunt” as used herein, refers to a metallic component configured to connect areas differing in magnetic polarity of the at least one stationary magnet 520, so as to divert part of the magnetic flux lines to pass through said magnetic shunt (illustrated as second flux lines 525), and therefore to decrease the fraction of magnetic flux lines passing through the rest of the capsule and extending outward from the capsule (illustrated as first flux lines 524).

According to some embodiments, the at least one magnetic shunt 575 is shaped as a curvilinear cylinder or a horseshoe. However, it is to be understood that the at least one magnetic shunt 575 fulfill the same function when otherwise shaped, as a ring, ovoid, ellipsoid, rectangle, square, or any other polyhedron. Each possibility represents a separate embodiment of the present invention. Moreover, the cross-sectional geometry of magnetic shunt 575 may be circular, triangular or any other curvilinear or rectilinear cross-section.

According to some embodiments, the at least one magnetic shunt 575 comprise a ferromagnetic compound, such as but not limited to, iron, iron oxide, neodymium iron boron, nickel, aluminum nickel cobalt, cobalt, samarium cobalt, and any combination thereof. Each possibility represents a separate embodiment of the present invention. In some embodiments, the at least one magnetic shunt 575 is made of a ferromagnetic compound comprising soft iron.

According to some embodiments, the at least one pharmaceutical composition 510 is in a solid dosage form, such as but not limited to, a pill, compressed powder, a tablet, a micro tablet, a granule, a combination thereof, or any other solid formulation known in the pharmaceutical art. Each possibility represents a separate embodiment of the present invention. In some embodiments, the at least one drug chamber 560 comprise a plurality of pharmaceutical compositions 510 disposed within. It is noted that the same componential and structural principles disclosed for pharmaceutical composition 310 similarly apply to pharmaceutical composition 510.

It is noted that the same functional and structural principles disclosed for the first opening 330 similarly apply to the at least one opening 530 which is located at the surface of the drug chamber 560, as presented at FIG. 7A.

Capsule 501 is manufactured to have a size and a shape appropriate for ingestion and for easy delivery through the various segments of the GI tract. It is noted that the same functional and structural principles disclosed for the size and the shape of capsule 301 similarly apply to capsule 501.

According to some embodiments, the at least one stationary magnet 520 is disposed within an inner surface of the capsule, as shown at FIG. 7A. In further embodiments, the stationary magnet 520 is in contact with, or attached to, the shortest face of the inner surface of the capsule 501, perpendicularly to the central longitudinal axis of capsule 501.

According to some embodiments, the stationary magnet 520 is cylinder shaped. However, it is to be understood that the stationary magnet 520 fulfill the same function when otherwise shaped, as a disc, ring, sphere, ovoid, ellipsoid, or any other polyhedron. Each possibility represents a separate embodiment of the present invention. It is noted that the same functional and structural principles disclosed for stationary magnet 320 similarly apply to stationary magnet 520.

According to some embodiments, the at least one stationary magnet 520 is configured to direct (or to capture and secure) the capsule 501 to a desired location of the GI tract, in response to the intensity of the magnetic field induced by at least one external magnet. According to some embodiments the at least one stationary magnet 520 is configured to secure the capsule 501 to the GI wall at a desired location in the GI tract.

In some embodiments, the capsule 501 is configured to transition between an open state and a closed state, wherein in the closed state the capsule 501 is secured to the GI wall so that the at least one opening 530 is sealed/obstructed thereby, thus preventing the pharmaceutical composition and/or the pharmaceutical active agents to exit the capsule, and wherein in the open state the capsule 501 is secured to the GI wall so that the pharmaceutical composition and/or the pharmaceutical active agents are released through the at least one opening 530, towards the interior of the GI tract. In further embodiments, in the closed state of the capsule 501 the at least one opening 530 faces the GI wall and is sealed/obstructed thereby. In further embodiments, in the open state of the capsule 501 the at least one opening 530 does not faces the GI wall and is facing the interior of the GI tract. In still further embodiments, in the open state of the capsule 501, preferably, the opening 530 is located on a surface of the capsule which is oppositely parallel to the surface which is secured against the GI wall.

Although not treated in full detail, it should be readily understood that the various capsules according to the various configurations described herein can transition between the open state and the closed state, described herein above.

In some embodiments, the capsule 501 is configured to allow fluid from the interior of the GI tract to enter the drug chamber 560 through the at least one opening 530, at a desired location in the GI tract. In further embodiments, the at least one pharmaceutical composition 510 is configured to controllably dissolve/erode over time upon coming into contact with fluid from the interior of the GI tract, and to release the pharmaceutical composition and/or the pharmaceutical active agents into the drug chamber 560. In still further embodiments, the capsule 501 is configured to release the pharmaceutical composition and/or pharmaceutical active agents over time, optionally by diffusion from the drug chamber 560 through the at least one opening 530, into the interior of the GI tract, driven out by a gradient in concentration, as disclosed herein above in the context of capsule 401.

The stationary magnet of the capsules disclosed herein, the capsules being administered per os to the GI tract, may form an internal magnetic attraction to the stationary magnet of a used capsule still residing in the GI tract, wherein said used capsule has already released its content at the desired location in the GI tract. Said internal magnetic attraction may affect or impair the drug release mechanism of pharmaceutical active agents from the capsule, as disclosed herein. It should be understood however, that the magnetic field intensity of the at least one external magnet is higher than the magnetic field intensity within the capsule, and therefore the internal magnetic attraction does not endanger the user.

In order to overcome this issue, the present inventors have developed a capsule comprising at least one magnetic shunt 575, wherein said magnetic shunt 575 is configured to reduce the intensity of the magnetic field induced by the at least one stationary magnet 520. In some embodiments, following the controlled release of an affective amount of the pharmaceutical composition and/or pharmaceutical active agents over time from capsule 501 into the interior of the GI tract, the at least one magnetic shunt 575 is configured to reduce the intensity of the internal magnetic field induced by the at least one stationary magnet 520. By reducing the intensity of the internal magnetic field induced by the stationary magnet 520, the danger of internal magnetic attraction between two or more stationary magnets originating from two or more capsules is diminished. Additionally, by reducing the intensity of the internal magnetic field, the magnetic attraction between the external magnet and the stationary magnet 520 will be decreased, and as a result capsule 501 will be able to be easily pass through and to be removed from the GI tract following the controlled release of an affective amount of pharmaceutical active agents.

As used herein, the term “affective amount” refers to the amount or quantity of pharmaceutical active agents sufficient to yield the desired therapeutic effect or response when administered to a user in need thereof, without undue adverse side effects (such as toxicity, irritation, or allergic response). The release of an affective amount of the pharmaceutical composition 510 from the capsule 501 into the interior of the GI tract follows the dissolution of the pharmaceutical composition 510 due to coming into contact with fluid from the GI tract.

According to some embodiments, the dissolution of the at least one pharmaceutical composition 510 allows the advancement of the at least one magnetic shunt 575 towards the at least one stationary magnet 520, due to magnetic attraction.

According to some embodiments, the at least one magnetic shunt 575 is configured to travel along the central longitudinal axis of capsule 501 towards the at least one stationary magnet 520 and to attach thereto, following the release of an affective amount of the pharmaceutical composition and/or pharmaceutical active agents from the capsule 501, as illustrated at FIG. 7B.

Once the at least one magnetic shunt 575 is attached to the at least one stationary magnet 520, the fraction of the magnetic flux originating from the at least one stationary magnet 520 and passing through the at least one magnetic shunt 575 is increased, wherein the total of said magnetic flux remains constant. As a result, the remaining fraction of the magnetic flux extending outward from the capsule is decreased, and therefore the intensity of the internal magnetic field induced by the at least one stationary magnet 520 which extends outwards from the capsule is reduced.

The amount of the illustrated flux lines is representative of the intensity of the magnetic field induced by the at least one stationary magnet 520. Therefore, the configuration of the embodiment illustrated in FIG. 7B results in a weaker internal magnetic field induced by the at least one stationary magnet 520 extending outward from the capsule, as indicated by a higher amount of illustrated second flux lines 525 passing through the at least one magnetic shunt 575, than compared to the configuration of the embodiment illustrated in FIG. 7A, depicting a stronger internal magnetic field induced by the at least one stationary magnet 520 extending outward from the capsule, as indicated by a lower amount of illustrated second flux lines 525 passing through the at least one magnetic shunt 575.

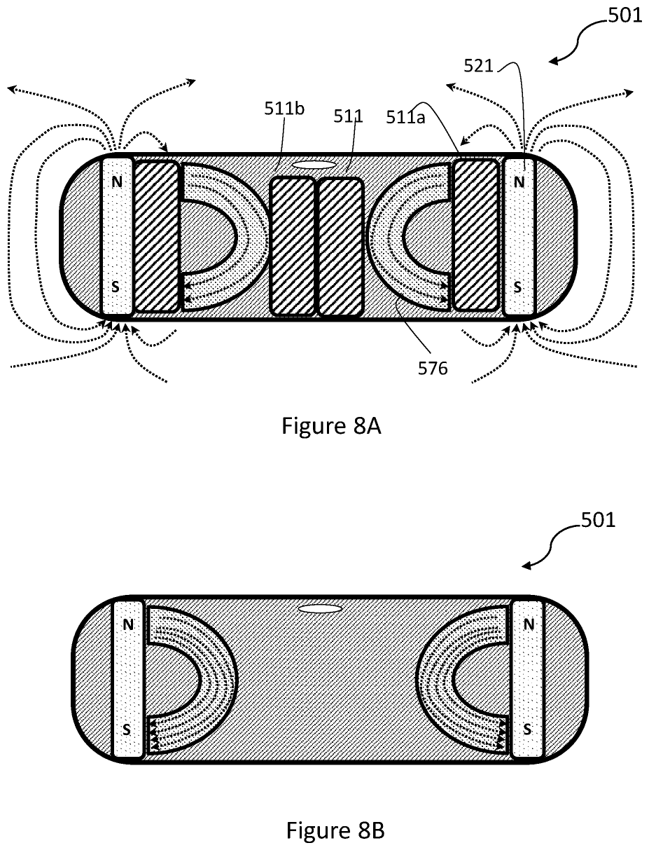

FIG. 8A illustrates another embodiment of the capsule 501 which differs from the embodiments of FIG. 7A in that: the at least one stationary magnet 520 comprises a first stationary magnet 520 and a second stationary magnet 521, wherein the first stationary magnet 520 and the second stationary magnet 521 are disposed within inner surfaces of the capsule, parallelly facing each other. In some embodiments, the second stationary magnet 521 is disposed within an inner surface of the capsule 501, parallelly facing the first stationary magnet 520, as shown at FIG. 8A. In further embodiments, the second stationary magnet 521 is in contact with, or attached to, the shortest face of the inner surface of the capsule 501, perpendicularly to the central longitudinal axis of capsule 501, parallelly facing the first stationary magnet 520.

In some embodiments, the at least one magnetic shunt 575 comprise a first magnetic shunt 575 and a second magnetic shunt 576. In further embodiments, the capsule 501 further comprise at least one additional pharmaceutical composition 511, wherein the at least one additional pharmaceutical composition 511 and the second magnetic shunt 576 are disposed within the drug chamber 560. In further embodiments, the capsule 501 further comprises a first additional pharmaceutical composition 511a, wherein the first additional pharmaceutical composition 511a is positioned between the second stationary magnet 521 and the second magnetic shunt 576, prior to the release of the pharmaceutical active agents from the capsule at the desired location in the GI tract.

According to some embodiments, the drug chamber 560 further comprise a plurality of additional pharmaceutical compositions 511. In further embodiments, the drug chamber 560 further comprise a second additional pharmaceutical composition 511b positioned between the first magnetic shunt 575 and the second magnetic shunt 576, thereby separating them from one another. In still further embodiments, the drug chamber 560 further comprise a plurality of additional pharmaceutical compositions 511 positioned between the first magnetic shunt 575 and the second magnetic shunt 576, thereby separating them from one another. In yet still further embodiments, the first magnetic shunt 575 is spaced from the second magnetic shunt 576 by a plurality of additional pharmaceutical compositions 511.

According to some embodiments, following the controlled release of an affective amount of the pharmaceutical composition and/or pharmaceutical active agents over time from the capsule 501, the first magnetic shunt 575 and the second magnetic shunt 576 are configured to travel along the central longitudinal axis of capsule 501 in opposite directions, until said first magnetic shunt 575 and second magnetic shunt 576 are attached to the first stationary magnet 520, and the second stationary magnet 521, respectively, as illustrated at FIG. 8B. According to further embodiments, following the controlled release of an affective amount of the pharmaceutical composition and/or pharmaceutical active agents over time from capsule 501 into the interior of the GI tract, the first magnetic shunt 575 and the second magnetic shunt 576 are configured to reduce the intensity of the internal magnetic fields induced by the first stationary magnet 520 and the second stationary magnet 521, respectively.

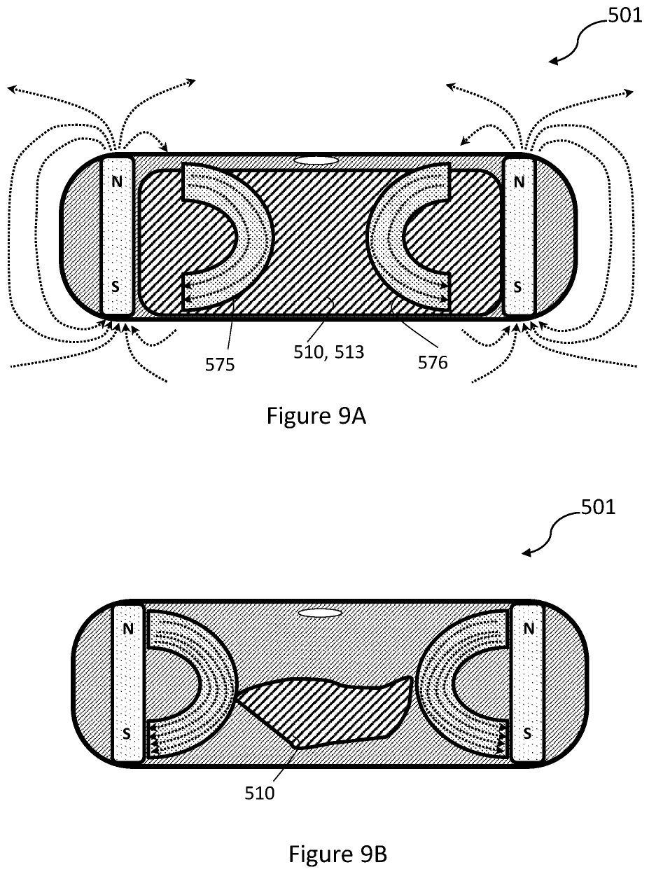

FIG. 9A illustrates another embodiment of the capsule 501 which differs from the embodiments of FIG. 8A in that the pharmaceutical composition 510 extends between the first stationary magnet 520 and the second stationary magnet 521, wherein the first magnetic shunt 575 and the second magnetic shunt 576 are disposed within the pharmaceutical composition 510 and are fixed thereto, so that to prevent contact between the shunts and the stationary magnets prior to the release of the pharmaceutical active agents from the capsule at the desired location in the GI tract. In further embodiments, the first magnetic shunt 575 and the second magnetic shunt 576 are disposed within the pharmaceutical composition 510 and are fixed thereto, so that to separate between the first stationary magnet 520 and the second stationary magnet 521 from the first magnetic shunt 575 and the second magnetic shunt 576, respectively, at the time of administration of capsule 501 to a user in need thereof, but prior to the release of pharmaceutical active agents from capsule 501 at a desired location in the GI tract.

According to some embodiments, following the controlled release of an affective amount of the pharmaceutical composition and/or pharmaceutical active agents over time from the capsule 501, the first magnetic shunt 575 and the second magnetic shunt 576 are configured to travel along the central longitudinal axis of capsule 501 in opposite directions, until said first magnetic shunt 575 and second magnetic shunt 576 are attached to the stationary magnet 520, and the second stationary magnet 521, respectively, as illustrated at FIG. 9B. According to further embodiments, following the controlled release of an affective amount of the pharmaceutical composition and/or pharmaceutical active agents over time from capsule 501 into the interior of the GI tract, the first magnetic shunt 575 and the second magnetic shunt 576 are configured to reduce the intensity of the internal magnetic fields induced by stationary magnet 520 and the second stationary magnet 521, respectively.

In some embodiments, the controlled release of an affective amount of pharmaceutical active agents over time result in the complete dissolution of the pharmaceutical composition 510. In some embodiments, the release of an affective amount of pharmaceutical active agents does not result in the complete dissolution of the pharmaceutical composition 510, and a fraction of pharmaceutical composition 510 remains disposed within the drug chamber 560, as illustrated at FIG. 9B.

Thus, responsive to an erosion of the at least one buffer 513 (i.e., the pharmaceutical composition 510), an intensity of the external output magnetic field of the at least one stationary magnet 520 (e.g., the first stationary magnet 520 and/or the second stationary magnet 521) is reduced. Particularly, each stationary magnet 520 outputs a magnetic field external to the capsule 501, as described above. The term “erosion”, as used herein, means that the respective at least one buffer 513, i.e. the respective pharmaceutical composition 510, is removed from its initial location in the capsule 501. This removal can be by being dissolved in the GI tract fluid, undergoing disintegration, or by any other mechanism known in the art. Therefore, as the at least one buffer 513 erodes, relative movement is initiated between the at least one stationary magnet 520 and the at least one magnetic shunt 575 such that the at least one magnetic shunt 575 approaches, and optionally contacts, the at least one fixed magnet 520. As a result, the portion of the magnetic field of the at least one stationary magnet 520 output external to the capsule 501 is reduced since it flows through the at least one magnetic shunt 575.

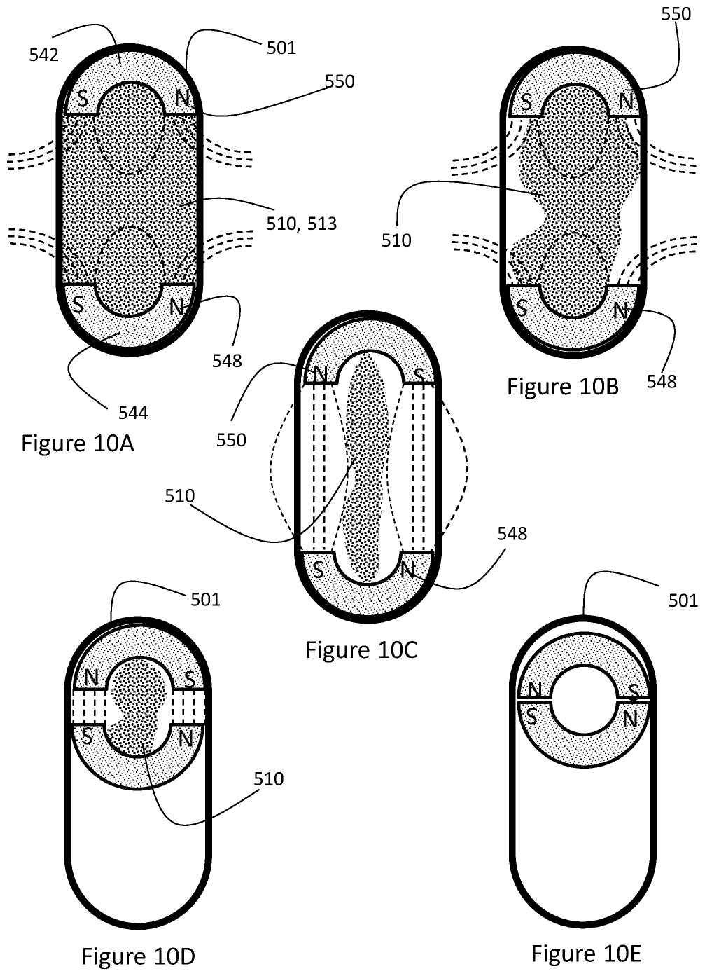

FIGS. 10A-10E illustrate an alternative embodiment of the capsule 501 which differs from the embodiments of FIG. 7A in that the capsule 501 comprises a pair of horse-shoe shaped magnets 542 and 544. According to some embodiments, the horse-shoe shaped magnets 542 and 544 are cylindrical. According to some embodiments, the magnets 542 and 544 are located at opposite ends of the capsule 501 and separated by a pharmaceutical composition 510 acting as a buffer 513. The pharmaceutical composition 510 extends between magnets 542 and 544, thereby preventing them from contacting each other prior to the release of the pharmaceutical composition 546 from the capsule 501 at the desired location in the GI tract. Additionally, pharmaceutical composition 510 maintains magnets 542 and 544 in a fixed orientation.

The north poles N 550 and 548 of magnets 542 and 544, respectively, are positioned at the same side of the capsule 501 such that the capsule 501 exhibits one side with north poles and one side with south poles, enabling an external magnet (not shown) to control movement of the capsule 501, as described above.

As pharmaceutical composition 510 is eroded, due to the release thereof through at least one opening in the capsule 501 (not shown), magnets 542 and 544 remain separated, thereby maintaining the angular orientation of poles 548 and 550. This is illustrated in FIG. 10B. As a result, the capsule 501 is still controlled by the external magnet, as described above.

As shown in FIG. 10C, as the pharmaceutical composition 510 continues to erode magnets 542 and 544 are released from the fixed orientation held by pharmaceutical composition 546. As a result, and due to the attraction between north poles 548 and 550, at least one of magnets 542 and 544 rotates so that its north pole faces the south pole of the other magnet. Due to the cylindrical shape of magnets 542 and 544, the walls of the capsule 501 allows magnets 542 and 544 only to rotate about the longitudinal axis of the capsule 501 and not along any other axis (e.g., perpendicular to the longitudinal axis). Thus, the magnetic flux lines of magnets 542 and 544 converge into a direct path between the poles, and the total magnetic field output from the capsule 501 weakens.

According to some embodiments, one of magnets 542 and 544 is a non-stationary magnet while the other magnet is a stationary magnet, wherein the non-stationary magnet is configured to move toward the stationary magnet, as a function of the erosion of the pharmaceutical composition 510, as disclosed herein. According to some embodiments, at least one of magnets 542 and 544 is configured to have the same functionalities and/or characteristics as the at least one stationary magnet 520, as disclosed herein above. According to some other embodiments, both magnets 542 and 544 are non-stationary magnets and are configured to move toward each other as a function of the erosion of the pharmaceutical composition 510 as disclosed herein.

As illustrated in FIG. 10D, as the pharmaceutical composition 510 further erodes, magnets 542 and 544 approach each other, thereby further weakening the magnetic flux. In such a case, the magnetic flux may be weak enough such that the capsule 501 is no longer held by the external magnet, thus flowing away with the stream in the GI. As illustrated in FIG. 10E, when the pharmaceutical composition 510 is fully eroded, magnets 542 and 544 come in contact with each other, thereby losing most of the magnetic attraction. Thus, when the capsule 501 passes through the GI on its way out of the body, it does not attract other empty capsules that may be there.

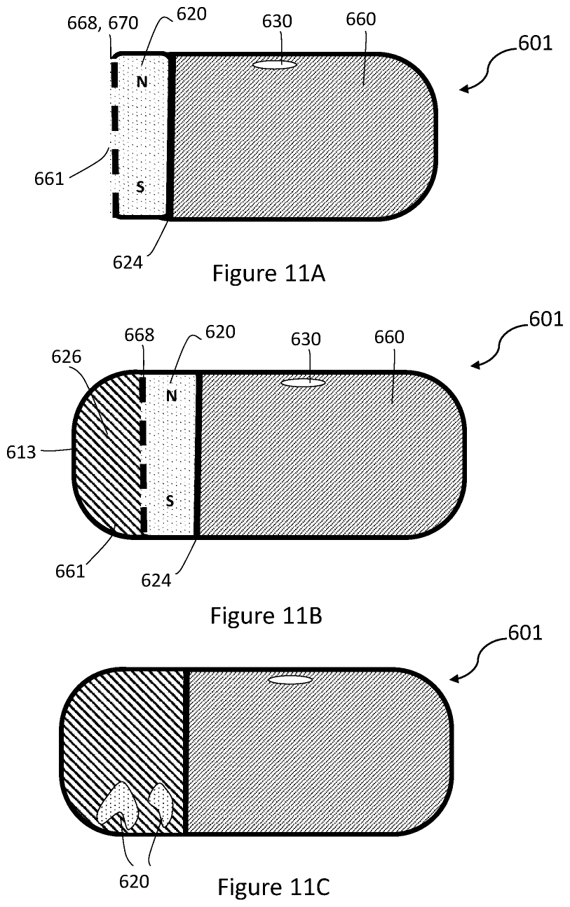

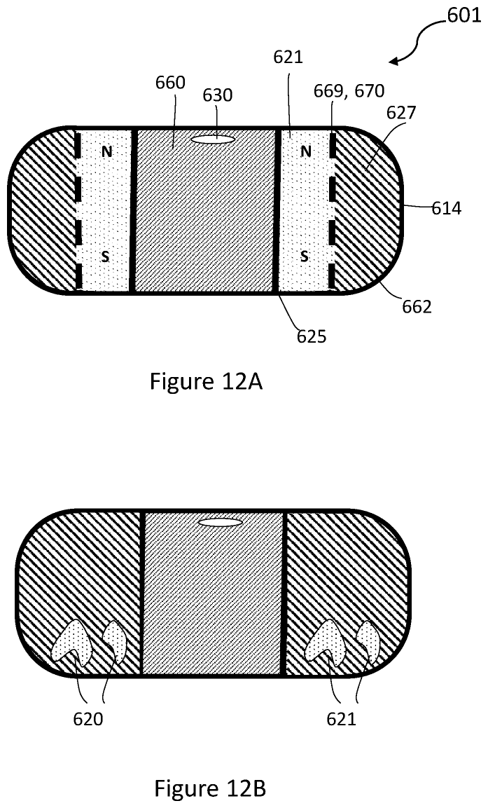

According to yet another aspect, there is provided a capsule 601. Reference is now made to FIGS. 11A-12B which constitute as cross-sectional views depicting capsule 601 through different embodiments of the present invention.

According to some embodiments, capsule 601 comprise: at least one drug chamber 660 comprising or exhibiting at least one opening 630; at least a first magnet chamber 661 comprising at least a first stationary magnet 620 disposed within and at least a first membrane 668 configured to at least partially cover said first stationary magnet 620; and a first magnet chamber barrier 624 having a first surface in contact with the at least a first stationary magnet 620 and a second surface in contact with the drug chamber 660, thereby separating the at least one drug chamber 660 from the at least a first magnet chamber 661, as illustrated at FIG. 11A.

In some embodiments, the first membrane 668 is configured to cover at least one surface of the first stationary magnet 620. In further embodiments, the first membrane 668 is configured to cover a plurality of surfaces of the first stationary magnet 620.

According to some embodiments, the at least a first stationary magnet 620 is located perpendicularly to the central longitudinal axis of capsule 601, and is disposed therebetween the first magnet chamber barrier 624 and the first membrane 668.

According to some embodiments, the at least a first membrane 668 comprise a biodegradable material, wherein said biodegradable material is configured to controllably degrade over time upon contact with the fluid from interior of the GI tract. Suitable biodegradable materials can be selected from: polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid), and combinations thereof. Each possibility represents a separate embodiment of the present invention.

According to some embodiments, the at least a first membrane 668 is configured to degrade over time and to expose the at least a first stationary magnet 620 to the fluid from the GI tract.

According to some embodiments, the at least a first magnet chamber 661 further comprises at least a first exterior surface 613 such that said at least first magnet chamber 661 extends between the first magnet chamber barrier 624 and the at least first exterior surface 613, wherein the at least first exterior surface 613 defines a first domain 626 within the first magnet chamber 661 extending from the at least first exterior surface 613 and the first membrane 668, as illustrated at FIG. 11B.

According to some embodiments, the first domain 626 is in fluid communication with the interior of the GI tract. In some embodiments, the at least a first exterior surface 613 comprise a porous material, configured to provide fluid communication between the first domain 626 and the GI tract. Said porous material can be made of a porous ceramic material or a porous aluminum oxide material. In some embodiments, the at least a first exterior surface 613 is configured to enable fluid from the interior of the GI tract to enter the first domain 626 through the pores of the porous material.

According to some embodiments, the at least a first membrane 668 is configured to degrade over time and to expose the at least a first stationary magnet 620 to the fluid from the GI tract, wherein said fluid is residing in the first domain 626.

It is noted that several of the functional and structural principles disclosed for stationary magnet 320 similarly apply to at least a first stationary magnet 620.

In some embodiments, the at least a first stationary magnet 620 does not comprise a corrosion-resistant coating. In further embodiments, the at least a first stationary magnet 620 is configured to undergo corrosion over time, following the exposure to the fluid from the interior of the GI tract. In still further embodiments, the at least a first stationary magnet 620 is configured to decompose or to disintegrate over time following the exposure to the fluid from the GI tract, as illustrated at FIG. 11C.

According to some embodiments, at least a first magnet chamber 661 further comprises a dry corrosive powder (not shown). In further embodiments, first domain 626 comprise said dry corrosive powder. In some embodiments, the dry corrosive powder is configured to form a corrosive solution upon exposure to fluids from the GI tract. In further embodiments, said corrosive solution is configured to corrode the at least a first stationary magnet 620. In still further embodiments, the at least a first stationary magnet 620 is configured to disintegrate upon exposure to the corrosive solution, hence controllably reduce over time the intensity of the internal magnetic field induced by the at least a first stationary magnet 620. Said corrosive powder can be selected from an acid, an oxidizer, a base, or a combination thereof.

As was disclosed herein above, the inventors discovered that the stationary magnet of a capsule administered to the GI tract may form an internal magnetic attraction to the stationary magnet of a used capsule still residing in the GI tract. In order to overcome this issue, the present inventors have developed an alternative capsule comprising at least one stationary magnet 620 configured to disintegrate upon exposure to fluids from the GI tract, and therefore reduce the internal magnetic field induced by the at least a first stationary magnet 620.

According to some embodiments, the at least a first stationary magnet 620 is configured to disintegrate upon exposure to fluids from the GI tract, hence controllably reduce over time the intensity of the internal magnetic field induced by the at least a first stationary magnet 620.

According to some embodiments, the at least a first magnet chamber 661 is configured to controllably expose the at least a first stationary magnet 620 to fluid from interior of the GI tract. In further embodiments, the at least a first stationary magnet 620 is configured to undergo corrosion over time following the exposure to the fluid from the interior of the GI tract, and therefore controllably reduce the intensity of the internal magnetic field induced by the at least a first stationary magnet 620.

According to some embodiments, drug chamber 660 is configured to contain therewithin a pharmaceutical composition 610 comprising at least one pharmaceutical active agent. According to some embodiments, drug chamber 660 further comprises at least one pharmaceutical unit comprising the pharmaceutical composition 610. It is noted that the same functional and structural principles disclosed for pharmaceutical composition 310 similarly apply to pharmaceutical composition 610.

It is noted that the same functional and structural principles disclosed for the first opening 330 similarly apply to the at least one opening 630 which is located on, or embedded within, the surface of the drug chamber 660, as presented at FIG. 11A.

Capsule 601 is manufactured to have a size and a shape appropriate for ingestion and for easy delivery through the various segments of the GI tract. It is noted that the same functional and structural principles disclosed for the size and the shape of capsule 301 similarly apply to capsule 601.

The drug chamber 660 may comprise a plurality of compartments, wherein at least one compartment is a drug compartment adapted to contain a pharmaceutical composition. It is to be understood that the drug compartment 660 may be filled with a pharmaceutical composition at the time of administration, prior to administration, and when being consumed by a subject but prior to its release from the capsule 601 at the desired location in the GI tract.

According to some embodiments, capsule 601 is configured for oral administration. In some embodiments, capsule 601 is configured for magnetically controlled drug release at a desired location of the GI tract. Said desired location in the GI tract can be selected from the duodenum, the jejunum, or the ileum.

According to some embodiments, capsule 601 further comprises a plurality of stationary magnets disposed within a plurality of magnet chambers, wherein each of the plurality of magnet chambers comprises a corresponding magnet chamber barrier.

FIG. 12A illustrates another embodiment of the capsule 601 which differs from the embodiments of FIG. 11A in that capsule 601 further comprises a second magnet chamber 662 parallelly facing the first magnet chamber 661, wherein said second magnet chamber 662 comprises a second stationary magnet 621 disposed within and a second membrane 669 configured to at least partially cover the second stationary magnet 621, and a second magnet chamber barrier 625 having a first surface in contact with the second stationary magnet 621 and a second surface in contact with the drug chamber 660, thereby separating the at least one drug chamber 660 from the second magnet chamber 662.

According to some embodiments, the second magnet chamber 662 further comprises a second exterior surface 614 such that the second magnet chamber 662 extends between the second magnet chamber barrier 625 and the second exterior surface 614, wherein the second exterior surface 614 defines a second domain 627 within the second magnet chamber 662 extending from the second exterior surface 614 and the second membrane 669, as illustrated at FIG. 12A.

According to some embodiments, the interior of the second domain 627 is in fluid communication with the GI tract. In some embodiments, the second exterior surface 614 comprise at least one opening disposed along the surface thereof (not shown). In further embodiments, the second exterior surface 614 comprise a plurality of openings disposed along surface thereof. Said opening are configured to allow fluid communication between the interior of the GI tract and the second domain 627.

It is noted that the same functional and structural principles disclosed for first the magnet chamber 661 similarly apply to the second magnet chamber 662.

In further embodiments, the first stationary magnet 620 and the second stationary magnet 621 are configured to undergo corrosion over time following the exposure to the fluid from the interior of the GI tract, and therefore controllably reduce the intensity of the internal magnetic fields induced by the first stationary magnet 620 and the second stationary magnet 621, respectively. In still further embodiments, the first stationary magnet 620 and the second stationary magnet 621 are configured to corrode, decompose and/or disintegrate over time following the exposure to the fluid from the interior of the GI tract, as illustrated at FIG. 12B.

Thus, the first membrane 668 and/or the second membrane 669 each constitute as a buffer 670 that separates the first stationary magnet 620 and/or the second stationary magnet 621 from an environment surrounding the capsule. As described above, the environment surrounding the capsule comprises GI fluid and the buffer 670 is configured to be eroded (e.g., to corrode, decompose and/or disintegrate) by the fluid of the GI tract. As further described above, the first stationary magnet 620 and the second stationary magnet 621 are eroded by the fluid of the GI tract responsive to the erosion of the at least one buffer 670 exposing the first stationary magnet 620 and the second stationary magnet 621 to the GI tract fluid. As described above, this can be further responsive to contact with a solution of a corrosive powder and the fluid of the GI tract, the solution formed when the first membrane 668 and/or the second membrane 669 erodes and the corrosive powder is exposed to the GI tract fluid. Thus, responsive to the erosion of the at least one buffer 670, the first stationary magnet 620 and the second stationary magnet 621 are eroded, thereby reducing the intensity of the output magnetic field thereof.

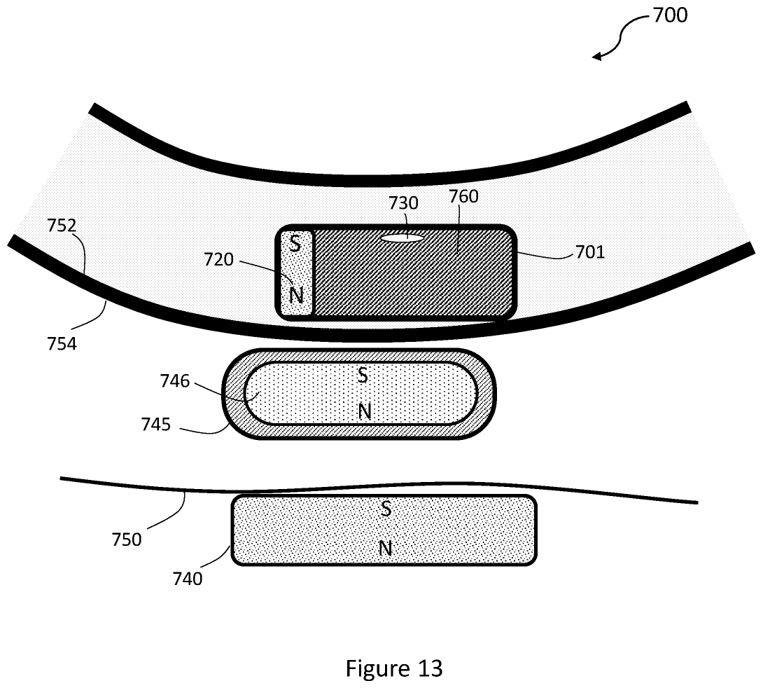

According to another aspect, there is provided a drug delivery system 700. Reference is now made to FIGS. 13-15. FIG. 13 constitutes a cross-sectional view depicting drug delivery system 700 through different embodiments of the present invention.

According to some embodiments, the drug delivery system 700 comprises: capsule 701 configured for oral administration and for magnetically controlled drug release at a desired location in the GI tract; at least one intermediate magnetic housing 745 comprising at least one inner magnet 746 disposed within, wherein the intermediate magnetic housing 745 is configured to be transplanted in a close vicinity to the external wall 754 of the GI tract at a desired location of the GI tract; and an external magnet kit comprising at least one external magnet 740, wherein the at least one external magnet 740 is configured to generate an external magnetic field and is further configured to be in contact with the skin of a user 750, at the vicinity of a desired location of the GI tract. Housing 745 and at least one inner magnet 746 are shaped and dimensioned such that at least one inner magnet 746 can rotate about itself, optionally about its longitudinal axis.

According to some embodiments, capsule 701 comprises at least one stationary magnet 720 disposed within and at least one drug chamber 760, wherein the at least one drug chamber 760 comprises at least one opening 730 and is adapted to contain therewithin a pharmaceutical composition. Although not treated in full detail, it should be readily understood that capsule 701 may combine various configurations according to any of the aspects and/or configurations described herein, with accordance to the principles of the present invention.

According to principles of the present invention, the at least one stationary magnet 720 is configured to direct the capsule 701 to an inner GI wall 752 at a desired location of the GI tract, in response to the external magnetic field, induced by the at least one external magnet 740. In further embodiments, the at least one external magnet 740 is configured to capture and to secure the capsule 701 at a desired location of the GI tract. In some embodiments, the at least one stationary magnet 720 is further configured to rotate the capsule, in response to a change in direction of the external magnetic field, induced by the at least one external magnet 740.

The present inventors have discovered that when the desired internal location in the GI tract is overly distant from the at least one external magnet 740 which is configured to be in contact with the skin of a user 750, the external magnetic field may not be strong enough to magnetically direct or rotate the at least one stationary magnet 720. As a result, the application of the controlled release mechanism of pharmaceutical active agents from the capsule 701 as was described herein above may be unobtainable.

As used herein, the term “overly distant” refers to a distance which can be larger than about 10 cm, optionally larger than about 15 cm, or alternately larger than about 20 cm.

One possible solution to said predicament is to increase the size of the at least one stationary magnet 720, however this may result in the manufacture of a heavier and larger capsule, making it inappropriate for ingestion and easy delivery through the various segments of the GI tract. Another possible solution is to increase the size of the at least one external magnet 740, however this may result in a heavier external magnet 740, making it more complicated to be in contact with the skin of a user 750, or form undesirable magnetic associations with the surrounding environment.

In order to successfully overcome this issue, the present inventors have developed a drug delivery system comprising at least one intermediate magnetic housing 745 comprising at least one inner magnet 746. According to some embodiments, the at least one inner magnet 746 is configured to control the magnetic field direction within the capsule 701, thereby controlling the flow of the pharmaceutical active agents from the capsule 701 to the GI tract.

According to further embodiments, the at least one inner magnet 746 is configured to capture and to secure the capsule 701 at a desired location of the GI tract, in response to the external magnetic field, induced by the at least one external magnet 740. Advantageously, the use of the at least one inner magnet 746 enables the manufacture of the capsule 701 having dimensions and a weight appropriate for ingestion and for easy delivery through the various segments of the GI tract, and further enables the at least one external magnet 740 to be successfully in contact with the skin of a user 750.

Capsule 701 is manufactured to have a size and a shape appropriate for ingestion and for easy delivery through the various segments of the GI tract. It is noted that the same functional and structural principles disclosed for the size and the shape of capsule 301 similarly apply to capsule 701.

In some embodiments, the at least one intermediate magnetic housing 745 is configured to be transplanted and/or implanted below the skin of the user. In further embodiments, the at least one intermediate magnetic housing 745 is configured to be transplanted and/or implanted in close vicinity to the external wall of the GI tract 754 at a desired location of the GI tract within the body of the user. In still further embodiments, the at least one intermediate magnetic housing 745 is transplanted in close vicinity to the external wall of the GI tract at the desired location of the GI tract utilizing laparoscopic surgery.

According to some embodiments, the at least one intermediate magnetic housing 745 is configured to be shaped as a cylinder. However, it is to be understood that the intermediate magnetic housing 745 fulfill the same function when otherwise shaped, as a sphere, ovoid, ellipsoid, square, rectangle, or any other polyhedron. Each possibility represents a separate embodiment of the present invention. In some embodiments, the at least one inner magnet 746 is configured to be shaped as a cylinder. However, it is to be understood that the inner magnet 746 fulfill the same function when otherwise shaped, as a sphere, ovoid, ellipsoid, square, rectangle, or any other polyhedron. Each possibility represents a separate embodiment of the present invention.

According to some embodiments, the central longitudinal axis of at least one intermediate magnetic housing 745 is located in parallel to the external wall of the GI tract 754 at the vicinity of a desired location of the GI tract, as schematically illustrates at FIG. 13.

According to some embodiments, the at least one inner magnet 746 is a permanent magnet, such as, but not limited to, ceramic, ferrite or Alnico magnets. In some embodiments, the at least one inner magnet 746 comprises at least one component selected from the group consisting of a ferromagnetic compound, a rare earth metal, a naturally occurring mineral, or any combination thereof In some embodiments, the ferromagnetic compound is selected from: iron, iron oxide, neodymium iron boron, nickel, aluminum nickel cobalt, cobalt, samarium cobalt, and any combination thereof. Each possibility represents a separate embodiment of the present invention. In some embodiments, the rare earth metal is selected from scandium, yttrium, lanthanide, and any combination thereof. Each possibility represents a separate embodiment of the present invention. The naturally occurring mineral can be lodestone.

According to some embodiments, the at least one inner magnet 746 is configured to have a diametrical direction of magnetic polarity. In further embodiments, the at least one inner magnet 746 is configured to rotate around its central longitudinal axis, in response to a change in direction of the external magnetic field, induced by the at least one external magnet 740.

According to some embodiments, the external magnet kit further comprise fastening means attached to the at least one external magnet 740, and configured to attach the at least one external magnet 740 to the body of a user. In some embodiments, the external magnet kit further comprise a control system configured to change the magnetic field direction of the at least one external magnet 740, thereby control the release of the pharmaceutical composition from the capsule 301 to the GI tract.

Without wishing to be bound by any theory or mechanism of action, it is contemplated that when the south magnetic pole of external magnet 740 is facing the outer layer of the skin 750 of a user, it magnetically attracts the north magnetic pole of the at least one inner magnet 746, which is configured to rotate around its central longitudinal axis, until its north magnetic pole is facing the south magnetic pole of external magnet 740. As a result, the north magnetic pole of the at least one stationary magnet 720 orients itself in the direction of the south magnetic pole of the inner magnet 746, and secure capsule 701 at a desired location of the GI tract.

Once capsule 701 arrives at the desired location of the GI tract, the at least one inner magnet 746 is configured to secure the capsule 701 to inner GI wall 752. When the external magnet 740 is rotated so that the north magnetic pole of external magnet 740 is facing the outer layer of the skin 750 of a user, it magnetically attracts the south magnetic pole of the at least one inner magnet 746. As a result, the at least one inner magnet 746 rotates around its central longitudinal axis in order to direct its south magnetic pole in the direction of external magnet 740. The rotation of the at least one inner magnet 746 causes the at least one stationary magnet 720 to rotate around its central longitudinal axis in order to direct its south magnetic pole in the direction of the at least one inner magnet 746. In some embodiments, the rotation of the at least one stationary magnet 720 is configured to rotate capsule 701 around its central longitudinal axis. The release mechanism of the pharmaceutical composition and/or the pharmaceutical active agents from the capsule 701 is similar to the release mechanism described herein above in accordance with other embodiments.

In some embodiments, the at least one inner magnet 746 is configured to control the flow of the pharmaceutical composition and/or pharmaceutical active agents from the capsule 701, as a function of changes in the external magnetic field induced by the at least one external magnet 740. In further embodiments, the at least one intermediate magnetic housing 745 is configured to be transplanted in close vicinity to the external wall of the GI tract 754 at the desired location in the GI tract, wherein said desired location is distanced over about 1 cm, over about 2 cm, over about 3 cm, or over about 5 cm, from the at least one external magnet 740, wherein said external magnet 740 is configured to be in contact with the skin of a user 750. In still further embodiments, the desired location is distanced over about 2 cm from the at least one external magnet 740.

In some embodiments, the capsule 701 is configured to controllably release the pharmaceutical composition and/or pharmaceutical active agents over time from the drug chamber 760 through the at least one opening 730, into the interior of the GI tract. As described above, in some embodiments the pharmaceutical composition and/or pharmaceutical active agents are controllably released due to a mobile element configured to push the pharmaceutical composition and/or pharmaceutical active agents towards at least one opening. As further described above, in some embodiments the pharmaceutical composition and/or pharmaceutical active agents are released by diffusion through the at least one opening.

In further embodiments, the at least one inner magnet 746 is configured to rotate around its central longitudinal axis, in response to a change in direction of the external magnetic field, induced by the at least one external magnet 740, wherein the rotation of the at least one inner magnet 746 is configured to rotate the capsule 701 around its central longitudinal axis.

Although not treated in full detail, it should be readily understood that capsule 701 can combine one or more of the configurations presented at FIGS. 7A-12A, wherein said configurations are designed to controllably reduce the intensity of the magnetic field induced by the at least one stationary magnet 720. In some embodiments, the capsule 701 is configured to controllably release the pharmaceutical composition and/or pharmaceutical active agents over time, optionally by diffusion, from the drug chamber 760 through the at least one opening 730 into the interior of the GI tract, wherein the capsule 701 comprises at least one configuration selected from the various embodiments of capsule 501 and/or capsule 601, as detailed herein above. By reducing the intensity of the magnetic field induced by the at least one stationary magnet 720, the magnetic attraction between the at least one inner magnet 746 and the at least one stationary magnet 720 will be decreased, and as a result capsule 701 will be able to be easily pass through the GI tract and to be removed from the body, following the controlled release of an affective amount of pharmaceutical active agents.

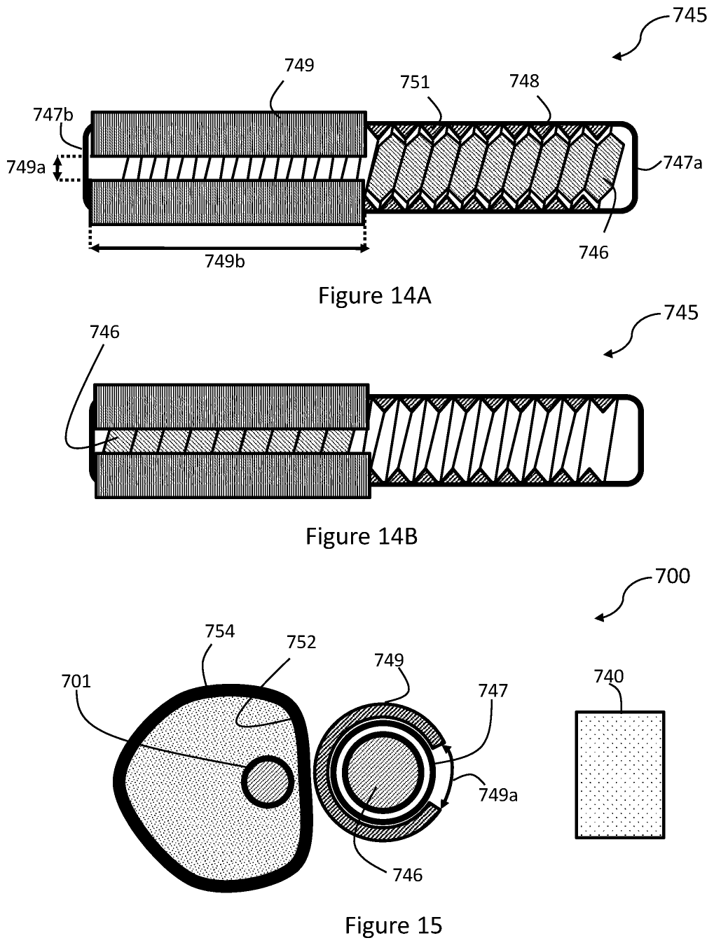

Reference is now made to FIGS. 14A-15. FIGS. 14A-14B constitutes side views depicting the at least one intermediate magnetic housing 745 presented at FIG. 13, from the perspective view of the at least one external magnet 740, through different embodiments of the present invention. FIG. 15 constitutes a top-view of a cross-sectional view of a drug delivery system 700 according to some embodiments.

FIGS. 14A-15 illustrates an embodiment of the at least one intermediate magnetic housing 745, comprising a translation member 751. According to some embodiments translation member 751 comprises a motor (not shown) and a threaded bore 748 which can extend along an inner surface of the at least one intermediate magnetic housing 745.In such an embodiment, the at least one inner magnet 746 is a threaded inner magnet 746 comprising a threaded surface, and the threaded bore 748 of translation member 751 is configured to receive, and threadedly engage with, the threaded inner magnet 746. According to some embodiments, a first portion of the at least one intermediate magnetic housing 745 is partially covered by a magnetic shield member 749 configured to reduce the magnetic field intensity of the threaded inner magnet 746 when the latter is at least partially covered by the magnetic shield member 749, and wherein the remaining uncovered area of the first portion is configured to face the at least one external magnet 740, thereby forming an uncovered portion 749a, as illustrated at FIG. 15.

Translation member 751 is configured to translate the at least one inner magnet 746 between a first position and a second position in relation to the magnetic shield member 749. According to some embodiments, the motor rotates the threaded inner magnet 746, which then advances along the threaded bore 748 towards the magnetic shield member 749. As described above, moving from the first position to the second position entails moving the at least one inner magnet 746 at least partially behind the magnetic shield member 749. Thus, the intensity of the magnetic field of the at least one inner magnet 746 is greater in the first position than in the second position, due to the magnetic shield member 749. At least one inner magnet 746 is illustrated in a first position in FIG. 14A as being completely outside of the magnetic shield member 749 and is illustrated in a second position in FIG. 14B as being completely inside the magnetic shield member 749, however this is not meant to be limiting in any way. According to some embodiments, in the first and/or second position the at least one inner magnet 746 is partially covered by the magnetic shield member 749 and partially not covered by the magnetic shield member 749, with a greater portion of the at least one inner magnet 746 covered by the magnetic shield member 749 in the second position than in the first position.

The threaded bore 748 extends from a first end 747a toward a second end 747b of the intermediate magnetic housing 745, while the magnetic shield member 749 extends from the second end 747b of the intermediate magnetic housing 745.

According to some embodiments, the length 749b of the magnetic shield member 749 along the central longitudinal axis of the intermediate magnetic housing 745 constitutes about 50% of the length of the intermediate magnetic housing 745, as illustrated at FIG. 14A.

According to some embodiments, the magnetic shield member 749 is configured to cover at least about 50% of the circumference of the first portion of the at least one intermediate magnetic housing 745. In further embodiments, the magnetic shield member 749 is configured to cover at least about 50%, about 60%, about 70%, about 80%, or about 90% of the circumference of the first portion of the at least one intermediate magnetic housing 745. In still further embodiments, the magnetic shield member 749 is configured to cover at least about 60% to about 90% of the circumference of the first portion of the at least one intermediate magnetic housing 745. In some exemplary embodiment, the magnetic shield member 749 is configured to cover at least about 70% to about 80% of the circumference of the first portion of the at least one intermediate magnetic housing 745.

According to some embodiments, the uncovered portion 749a constitutes less than about 50% of the circumference of the first portion of the at least one intermediate magnetic housing 745. In further embodiments, the uncovered portion 749a constitutes less than about 50%, about 40%, about 30%, about 20%, about 10%, or about 5% of the circumference of the first portion of the at least one intermediate magnetic housing 745. In still further embodiments, the uncovered portion 749a constitutes about 40% to about 10% of the circumference of the first portion of the at least one intermediate magnetic housing 745. In some exemplary embodiment, the uncovered portion 749a constitutes about 30% to about 20% of the circumference of the first portion of the at least one intermediate magnetic housing 745.

According to some embodiments, the magnetic shield member 749 comprise a material selected from the group consisting of a sheet metal, metal screen, and metal foam. The metal can be selected from copper, nickel, stainless steel, aluminum, bras, and combinations thereof.

Without wishing to being bound by any theory or mechanism of action, it is contemplated that since the uncovered portion 749a constitutes as an opening in the magnetic shield member 749 and is facing the at least one external magnet 740, the uncovered portion 749a enables the threaded inner magnet 746 to respond to the external magnetic field induced by the at least one external magnet 740 without being affected by the magnetic shield member 749, as illustrated at FIG. 15.

According to some embodiments, the uncovered portion 749a is configured to allow a magnetic field of the at least one external magnet 740 to cause the threaded inner magnet 746 to threadedly rotate inside threaded bore 748, between the first end 747a and the second end 747b of the intermediate magnetic housing 745. According to some embodiments, in response to a change in direction of the external magnetic field, induced by the at least one external magnet 740, the threaded inner magnet 746 changes the direction of rotation inside the threaded bore 748. According to some embodiments, in order to insert the threaded inner magnet 746 into the magnetic shield member 749 the external magnetic field of the at least one external magnet 740 is set in a first direction until at least a predetermined portion of the threaded inner magnet 746 enters the magnetic shield member 749. Similarly, according to some embodiments, in order to remove the threaded inner magnet 746 from the magnetic shield member 749 the external magnetic field of the at least one external magnet 740 is set in a second direction until at least the predetermined portion of the threaded inner magnet 746 exits the magnetic shield member 749. According to some embodiments, when the threaded inner magnet 746 is outside of the magnetic shield member 749, i.e. not being blocked by the magnetic shield member 749, the direction of the external magnetic field of the at least one external magnet 740 repeatedly changes between the first direction and the second direction, thereby causing the threaded inner magnet 746 to repeatedly change the direction of the magnetic field thereof. Particularly, repetitive changes in the magnetic field cause the stationary magnet 720 of the capsule 701 to repeatedly rotate the capsule 701, thereby alternating the capsule 701 between an open state where at least one opening 730 is not obstructed by inner GI wall 752 and a closed state where at least one opening 730 is obstructed by inner GI wall 752. In further embodiments, the uncovered portion 749a is configured to allow the threaded inner magnet 746 to threadedly rotate without being affected by the magnetic shield member 749. In still further embodiments, the magnetic shield member 749 does not affect the rotation of the threaded inner magnet 746 in response to a change in direction of the external magnetic field, induced by the at least one external magnet 740.

According to some embodiments, the threaded inner magnet 746 is configured to threadedly rotate inside the threaded bore 748, between the first end 747a and the second end 747b of the intermediate magnetic housing 745, in response to a change in direction of the external magnetic field, induced by the at least one external magnet 740, wherein the rotation of the threaded inner magnet 746 is configured to rotate the capsule 701 around its central longitudinal axis. Without wishing to being bound by any theory or mechanism of action, it is contemplated that the threaded rotation of the threaded inner magnet 746 by 180° or half a turn is configured to rotate the capsule 701 around its central longitudinal axis by 180°, and as a result to control the flow of pharmaceutical active agents from the capsule 701.

According to some embodiments, when the threaded inner magnet 746 is threading within the threaded bore 748 in the vicinity of the first end 747a, and wherein less than about 50% of the length of the threaded inner magnet 746 is covered by the magnetic shield member 749, as illustrated at FIG. 14A, the threaded inner magnet 746 is configured to rotate the capsule 701 and to control the flow of pharmaceutical active agents from the capsule 701, in response to a change in direction of the external magnetic field, induced by the at least one external magnet 740.

According to some embodiments, when the threaded inner magnet 746 is threading within threaded bore 748 in the vicinity of the second end 747b, and wherein more than about 50% of the length of the threaded inner magnet 746 is covered by the magnetic shield member 749, as illustrated at FIG. 14B, the threaded inner magnet 746 is unable to rotate or control the capsule 701. In further embodiments, when more than about 70% of the length of the threaded inner magnet 746 is covered by the magnetic shield member 749, the threaded inner magnet 746 is unable to secure the capsule 701 to inner GI wall 752.

According to some embodiments, the magnetic shield member 749 is configured to shield the capsule 701 from the magnetic field induced by the threaded inner magnet 746, wherein more than about 50% of the threaded inner magnet 746 is covered by the magnetic shield member 749. In further embodiments, the magnetic shield member 749 is configured to block or to reduce the magnetic field intensity induced by the threaded inner magnet 746, wherein more than about 50% of the threaded inner magnet 746 is covered by the magnetic shield member 749.

Advantageously, the inventors developed an intermediate magnetic housing 745 comprising a threaded inner magnet 746 configured to controllably reduce the intensity of the magnetic field induced by the threaded inner magnet 746, and therefore enable the capsule 701 to be removed from the GI tract. According to some embodiments, following the release of an affective amount of the pharmaceutical composition and/or pharmaceutical active agents from the capsule 701, the threaded inner magnet 746 is configured to controllably reduce the intensity of the magnetic field induced by the threaded inner magnet 746, in response to a change in direction of the external magnetic field, induced by the at least one external magnet 740. In further embodiments, by reducing the intensity of the magnetic field induced by the threaded inner magnet 746, the capsule 701 is no longer secured to the GI wall 752 and can be carried out of the body through the GI tract, via peristaltic movement of the GI tract and/or by the fluids flowing along the GI tract.

According to some embodiments, the intermediate magnetic housing 745 is configured to controllably reduce or amplify the intensity of the magnetic field induced by the threaded inner magnet 746. In further such embodiments, the capsule 701 can combine one or more of the configurations as presented herein above at FIGS. 3A-12A.

According to some embodiments, there is provided a method of treating a disease or disorder in a subject in need thereof, the method comprises:
providing system 700, wherein drug chamber 760 further comprises at least one pharmaceutical composition comprising at least one pharmaceutical active agent for treating the disease or disorder;
placing the intermediate magnetic housing 745 in a close vicinity to the external wall of the GI tract at a desired location of the GI tract, the intermediate magnetic housing 745 comprising an inner magnet 746;
attaching the external magnet kit comprising the at least one external magnet 740 to the skin of the subject; and
administering, per os, capsule 701.

In some embodiments, the method comprises attaching the external magnet kit comprising the external magnet 740 externally against the skin of a user, at the vicinity of a desired location of the GI tract. In further embodiments, the method further comprises placing the intermediate magnetic housing 745 against the external wall of the GI tract at a desired location of the GI tract, in which the pharmaceutical composition and/or the pharmaceutically active agents is to be released, utilizing laparoscopic surgery. In still further embodiments, the method further comprises directing the capsule to the desired location of the GI tract utilizing the intermediate magnetic housing 745, wherein the inner magnet 746 of the intermediate magnetic housing 745 is configured to respond to changes in direction of the external magnetic field, induced by the at least one external magnet. In yet still further embodiments, the method further comprises rotating the capsule and releasing the pharmaceutical composition and/or the pharmaceutically active agents at the desired location of the GI tract, utilizing the inner magnet 746 of the intermediate magnetic housing 745. In still further embodiments, the method further comprises controllably reducing the intensity of the magnetic field originating from the intermediate magnetic housing 745, following the release of an affective amount of pharmaceutical active agents from the capsule 701, in order to allow the capsule 701 to be removed from the GI tract and to leave the body of the subject.

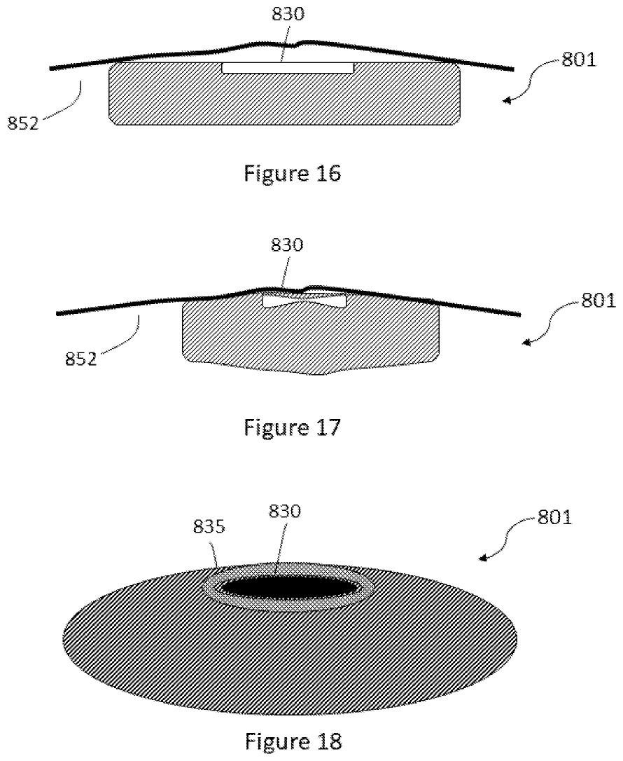

According to another aspect, there is provided a capsule 801. Reference is now made to FIGS. 16-18. FIGS. 16-18 constitutes a view in perspective of capsule 801 through different embodiments of the present invention. According to some embodiments, capsule 801 comprise at least one stationary magnet disposed within and at least one drug chamber comprising at least one opening 830. According to some embodiments, the drug chamber is configured to contain therewithin a pharmaceutical composition. According to some embodiments, the drug chamber further comprises at least one pharmaceutical composition comprising at least one pharmaceutical active agent. In further embodiments, capsule 801 is configured to be directed and/or secured to the inner GI wall 852 at a desired location of the GI tract. In still further embodiments, the at least one stationary magnet is configured to rotate the capsule 801 along its central axis, in proximity to the GI wall 852, in response to a change in direction of an external magnetic field, induced by at least one external magnet, as described above.

Although not treated in full detail, it should be readily understood that capsule 801 may combine various configurations and/or aspects of the capsules described herein with accordance to the principles of the present invention.

FIG. 16 illustrates capsule 801 secured to the inner GI wall 852, wherein the capsule 801 is shaped as a cylinder having hemispherical ends. Since the GI tract contain various segments varying in their diameter and circular shape, the surface of the inner GI wall 852 may contain various curvilinear segments. As a result, the cylindric capsule 801 is able to form direct contact with the curvilinear inner GI wall 852 solely by the two hemispherical edges of the capsule 801 in the shortest dimensions thereof, and not by the entire external surface of the capsule 801 facing the GI wall 852, as illustrated at FIG. 16. When the capsule is rotated so that the at least one opening 830 is facing the curvilinear inner GI wall 852, the at least one opening 830 is spaced from the curvilinear inner GI wall.

Since an adequate seal of the at least one opening 830 by the inner GI wall 852 is not possible due to the difference between the external geometrical shape of the capsule 801 and the texture of the inner GI wall 852, the pharmaceutical composition may be able to leak from the drug chamber in this configuration.

The leakage of the pharmaceutical composition from the drug chamber can occur by at least one of the following flowing mechanisms:
1. Gravitational mechanism: when fluid from the interior of the GI tract enters the drug chamber it starts to dissolve the pharmaceutical composition and to release the pharmaceutical composition into the drug chamber. If the density of the dissolved pharmaceutical composition is larger than the density of the fluid residing in the GI tract, than the pharmaceutical composition will sink in the direction of the gravitational force, and settle at the bottom of the capsule or the inner GI wall, depending on the location of the at least one opening 830 relatively to the desired location in the GI tract.
2. Diffusion mechanism: the pharmaceutical composition flow from the drug chamber towards the interior of the GI tract driven by the gradient in concentration.
3. Erosion mechanism: when fluid flows along the interior of the GI tract around the capsule 801 it can erode or carry away the pharmaceutical composition from the drug chamber along the GI tract.

It is contemplated that when there is no adequate seal of the at least one opening 830 by the inner GI wall 852, all three flowing mechanisms can contribute to the leakage of the pharmaceutical composition from the drug chamber.

FIG. 17 illustrates another embodiment of the capsule 801 which differs from the embodiments of FIG. 16 in that the capsule 801 comprise at least one curved surface, wherein the at least one opening 830 is located at the curved surface. In further embodiments, the cross-sectional geometry of said surface is selected from a circular, elliptic, or any other curvilinear cross-section shape.

According to some embodiments, the geometry of the capsule 801 is selected from a barrel-like shape, sphere, ellipsoid, cylinder, or any other circular geometric shape. According to some embodiments, capsule 801 further comprises an external housing configured to encompass the capsule 801. According to further embodiments, the geometry of the external housing of capsule 801 is selected from a barrel-like shape, sphere, ellipsoid, or any other circular geometric shape.

According to some embodiments, the capsule 801 is shaped as a barrel. According to some embodiment, the external housing of the capsule 801 is shaped as a barrel.

As used herein, the term barrel refers to a shape of the capsule 801 in which the diameter of the shape in the center thereof is larger relative to the diameter of each of the edges of the capsule.

Adventitiously, the present inventors have developed a capsule shaped as a barrel, or optionally as a sphere, ellipsoid, or any other circular geometric shape, so that when the capsule is rotated so that the at least one opening 830 is facing the curvilinear inner GI wall 852, the at least one opening 830 is adequately sealed against the curvilinear inner GI wall 852, as illustrated at FIG. 17. As a result, the pharmaceutical composition is prevented from exiting the drug chamber, regardless of the flowing mechanisms described herein above.

FIG. 18 illustrates another embodiment of the capsule 801 which differs from the embodiments of FIG. 17 in that the capsule further comprises a circumferential external barrier 835 surrounding the opening 830, and adapted to contact the GI wall when the capsule 801 is in use.

In further embodiments, the circumferential external barrier 835 is surrounding the opening 830 and is extending from the opening 830 in the vertical direction towards the outer environment (e.g., the GI tract).

According to some exemplary embodiment, the geometry of the capsule 801 is selected from a barrel-like shape, sphere, ellipsoid, or any other circular geometric shape, and the capsule further comprises a circumferential external barrier 835 surrounding the opening 830, and adapted to contact the GI wall when the capsule 801 is in use.

Adventitiously, the present inventors have a developed a capsule comprising a circumferential external barrier 835, so that when the capsule is rotated so that the at least one opening 830 is facing the curvilinear inner GI wall 852, the at least one opening 830 is adequately sealed between the circumferential external barrier 835 and the curvilinear inner GI wall 852. As a result, the pharmaceutical composition is prevented from exiting the drug chamber, regardless of the flowing mechanisms described herein above.

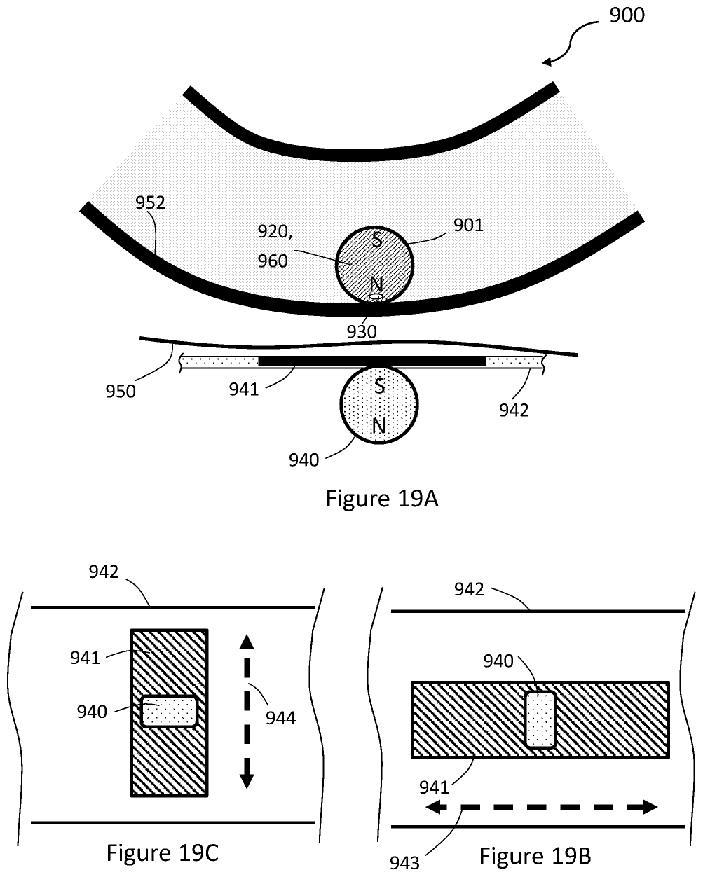

According to another aspect, there is provided a drug delivery system 900. Reference is now made to FIGS. 19A-19C. FIG. 19A constitutes a cross-sectional view depicting drug delivery system 900 through different embodiments of the present invention. FIG. 19B constitutes a side view depicting different configurations of rail 941 along a partial view of fastening means 942 through different embodiments of the present invention. FIG. 19C constitutes a side view depicting different configurations of rail 941 along a partial view of fastening means 942 through different embodiments of the present invention.

According to some embodiments, the drug delivery system 900 comprises: capsule 901 configured for oral administration and for magnetically controlled drug release at a desired location in the GI tract; and an external magnet kit comprising at least one external magnet 940 in contact with a rail 941 configured to support movement of the at least one external magnet 940 along the rail, wherein the at least one external magnet 940 is configured to generate an external magnetic field.

According to some embodiments, the geometry of capsule 901 is selected from a barrel-like shape, sphere-like shape, an ellipsoid, a spherocylinder, or any other curvilinear geometric shape. According to further embodiments, capsule 901 is in the shape of an ellipsoid or a spherocylinder. According to further embodiments, capsule 901 is in the shape of a cylinder having hemispherical ends.

As used herein, the term “sphere-like shape” refers to the geometric shape of the capsule 901 having a substantially spherical shape. Said shape can be a round sphere, i.e. a perfectly round ball, as illustrated at FIG. 19A. Alternately, said shape can be an ellipsoid, i.e. a quadric surface having planar cross sections in the shape of ellipses (not shown).

According to some embodiments, the at least one external magnet 940 is cylinder shaped. However, it is to be understood that the at least one external magnet 940 fulfills the same function when otherwise shaped, as a sphere, ovoid, ellipsoid, spherocylinder, or any other polyhedron. Each possibility represents a separate embodiment of the present invention. In some embodiments, the at least one external magnet 940 is configured to have a diametrical direction of magnetic polarity.

According to some embodiments, the external magnet kit further comprises a fastening means 942 configured to attach the rail 941 to the skin. In further embodiments, rail 941 comprise a flexible material, selected from a polymer and/or a sheet metal. In still further embodiments, the shape of the rail 941 is adapted to match the geometric shape of the skin of a user. In some embodiments, the external magnet kit further comprise a control system configured to change the magnetic field direction of the at least one external magnet 940, thereby control the release of the pharmaceutical composition from the capsule 901 to the GI tract.

In some embodiments, the at least one external magnet 940 is configured to rotate around itself or to roll along rail 941.

FIG. 19B constitutes a side view depicting a configuration of rail 941 along a partial view of fastening means 942. In some embodiments, rail 941 is positioned in parallel to a longitudinal axis 943, wherein the at least one external magnet 940 is configured to rotate around itself along rail 941, in the directions of the longitudinal axis 943.

FIG. 19C constitutes a side view depicting a configuration of rail 941 along a partial view of fastening means 942. In some embodiments, rail 941 is positioned in parallel to a vertical axis 944, located perpendicularly to the longitudinal axis 943, wherein the at least one external magnet 940 is configured to rotate around itself along rail 941, in the directions of the vertical axis 944.

In some embodiments, rail 941 comprise a longitudinal portion and a vertical portion intersecting in a middle portion thereof (not shown), wherein the at least one external magnet 940 is configured to rotate around itself while being translated along the longitudinal portion of the rail 941 and while being translated along the vertical portion of the rail 941. In further embodiments, the external magnet 940 can rotate around itself while simultaneously being translated in the direction of the longitudinal axis 943 and in the direction of the vertical axis 944. In further such embodiments, rail 941 is configured to be in a shape selected from X-shaped or plus(+)-shaped (not shown). In some embodiments (not shown), external magnet 940 rotates around itself responsive to a rotating rod extending through the center thereof, the rotating rod rotated by a motor. In other embodiments (not shown), a mechanism is provided that is configured to push external magnet 940, thereby rolling it along rail 941.

According to some embodiments, rail 941 is attached to fastening means 942 through a rod (not shown). In some embodiments, the rod is configured to enable rotation of rail 941, around itself, in parallel to fastening means 942, thereby causing the at least one external magnet 940 to rotate around itself, while allowing lateral movement along rail 941 in a plurality of directions.

In some embodiments, rail 941 is configured to be in the shape of a circle or an ellipse (not shown).

In some embodiments, the at least one external magnet 940 is configured to rotate around its central longitudinal axis, along rail 941, in parallel to the skin of a user 950. In some embodiments, the at least one external magnet 940 rotates along rail 941 at a frequency ranging from about 0.5 to about 20 rotations per minute. In further embodiments, the at least one external magnet 940 rotates along rail 941 at a frequency of about 1 rotation per minute.

In some embodiments, the at least one external magnet 940 is cylinder shaped and has a diameter in the range of about 1 to about 50 mm. In further embodiments, the diameter ranges between about 1 mm to about 20 mm. In still further embodiments, the diameter ranges between about 5 mm to about 15 mm. In yet still further embodiments, the diameter ranges between about 7 mm to about 12 mm.

According to some embodiments, the length of rail 941 ranges from about 1 cm to about 20 cm. In further embodiments, the length of rail 941 ranges from about 5 cm to about 15 cm. In still further embodiments, the length of rail 941 ranges from about 8 cm to about 12 cm.

According to some embodiments, the geometry of capsule 901 is a sphere-like shape. According to some embodiments, the diameter of capsule 901 ranges from about 1 mm to about 15 mm. In further embodiments, the diameter of capsule 901 ranges from about 3 mm to about 13 mm. In still further embodiments, the diameter of capsule 901 ranges from about 5 mm to about 10 mm.

According to some embodiments, as described above in relation to capsules 301, 301^{a}, 401, 501, 601, 701 and 801, capsule 901 comprises at least one stationary magnet 920 disposed within the at least one drug chamber 960, wherein the at least one drug chamber 960 comprises at least one opening 930 and is adapted to contain therewithin a pharmaceutical composition. Although not treated in full detail, it should be readily understood that capsule 901 may combine various configurations according to any of the aspects and/or configurations described herein, with accordance to the principles of the present invention.

According to some embodiments, the rotation of the at least one external magnet 940 along rail 941 is configured to induce a change in the direction of the external magnetic field, as was explained herein above, for example with regards to the external magnet 340. In further embodiments, the direction of the magnetic field of the at least one external magnet 940 is responsive to the roll thereof along the rail 941. In further embodiments, the at least one stationary magnet is configured to rotate the capsule 901 along the curvilinear inner GI wall 952, in response to the change in the direction of the external magnetic field, induced by the rotation of the at least one external magnet 940. In still further embodiments, the at least one stationary magnet is configured to capture and to secure the capsule 901 at a desired location of the GI tract, in response to the rotation of the at least one external magnet 940. In yet still further embodiments, the capsule 901 is configured to controllably release the pharmaceutical composition over time from the drug chamber through the at least one opening, into the interior of the GI tract, in response to the rotation of the at least one external magnet 940.

Advantageously, the rotation of capsule 901 along the inner GI wall cannot graze or damage the surface of the curvilinear inner GI wall 952, due to the relatively small contact area between the curvilinear inner GI wall 952 and the capsule 901, resulting from the curvilinear geometric shape of capsule 901, such as but not limited to a barrel-like shape, sphere-like, an ellipsoid geometrical shape and a spherocylinder geometrical shape.

Additionally, when the capsule 901 is rotated so that the at least one opening 930 is facing the curvilinear inner GI wall 952, the at least one opening is adequately sealed against the curvilinear inner GI wall 952, as illustrated at FIG. 19A. Advantageously, the rotation of curvilinear capsule 901 along the curvilinear inner GI wall 952 can prevent from the pharmaceutical composition to leak uncontrollably from the drug chamber.

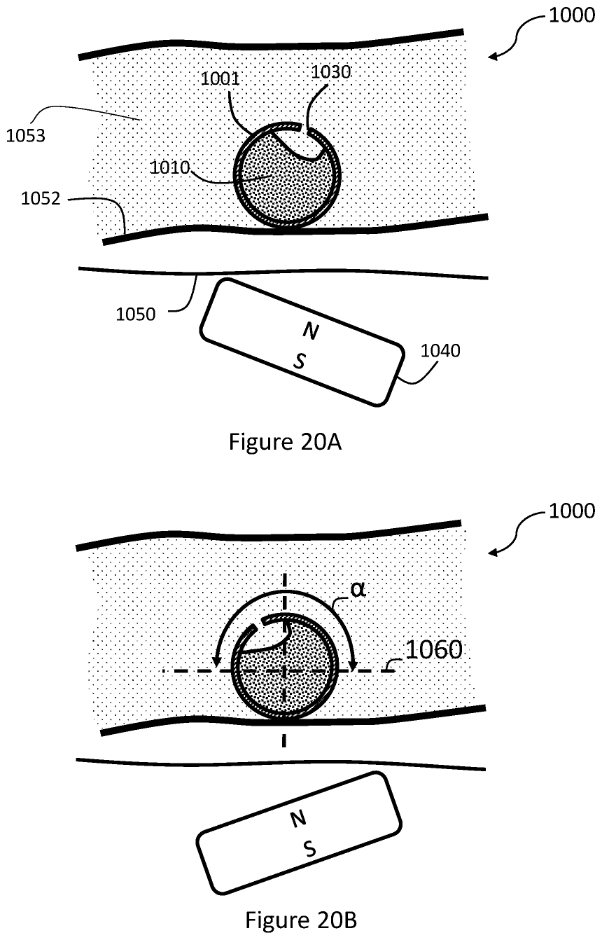

According to yet another aspect, there is provided a method for expedited release of a pharmaceutical composition from a capsule configured for magnetically controlled drug release. Reference is now made to FIGS. 20A-20B, which constitutes a top-view of a cross-sectional view depicting drug delivery system 1000 through different embodiments of the present invention.

According to some embodiments, there is provided a method for expedited release of a pharmaceutical composition from a capsule configured for magnetically controlled drug release, the method comprises:
a) providing drug delivery system 1000 comprising:
   a capsule 1001 configured for oral administration and for magnetically controlled drug release of a pharmaceutical composition 1010 at a desired location in the GI tract, comprising at least one opening 1030 and at least one internal magnet; and
   an external magnet kit comprising at least one external magnet 1040, wherein the at least one external magnet 1040 is configured to generate an external magnetic field and is further configured to be in contact with the skin of a user 1050, at the vicinity of a desired location of the GI tract;
b) attaching and/or juxtaposing the at least one external magnet 1040 to the skin of a user 1050 at the vicinity of a desired location of the GI tract;
c) administering, per os, capsule 1001;
d) directing the capsule 1001 to the desired location of the GI tract by controlling the intensity and/or direction of the magnetic field induced by the at least one external magnet 1040, thereby securing the administered capsule 1001 to the desired location of the GI tract responsive to the magnetic field of the at least one external magnet 1040; and
e) generating a change in the direction of the magnetic field induced by the at least one external magnet 1040 thereby inducing rotation of the capsule 1001 such that the at least one opening 1030 faces the interior of the GI tract 1053, due to the orientation of the at least one internal magnet, thereby releasing the pharmaceutical composition 1010 from said at least one opening 1030 at the desired location of the GI tract.

According to some embodiments, the method further comprises step (g) of continuously altering (or repeatedly adjusting) the direction of the magnetic field induced by the at least one external magnet 1040, following step (e), thereby shaking the capsule 1001 and accelerating the release rate of the pharmaceutical composition 1010 from said at least one opening 1030.

As used herein, the term “shaking the capsule” refers to generating continuous changes in the direction of the magnetic field induced by the at least one external magnet 1040, such that the capsule 1001 is rotated or tilted back and forth (i.e., in a clockwise direction and a counter-clockwise direction) along the inner GI wall 1052, at a rotation angle α at a cartesian coordinate system 1060, wherein said rotation angle α is ranging from about 0° to about 360°. In some embodiments, repeatedly rotating or adjusting the at least one external magnet 1040 alternately in a clockwise direction and a counter-clockwise direction, causes the adjusting of the direction of the magnetic field thereof.

In some embodiments, generating continuous changes in the direction of the magnetic field induced by the at least one external magnet 1040 comprise rotating the at least one external magnet 1040 by the rotation angle a, as illustrated at FIGS. 20A-20B.

According to some embodiments, the rotation angle α ranges between about 1° to about 180°. In further embodiments, the rotation angle α ranges between about 20° to about 160°.

Advantageously, the method disclosed herein provides expedited release of pharmaceutical active agents from capsule 1001, achieved by the shaking of the capsule 1001 according to step (g) which can accelerate the erosion of the pharmaceutical active agents from the capsule 1001 to the GI tract, in some embodiments.

According to some embodiments, the method further comprises step (h) of reversing the direction of the magnetic field induced by the at least one external magnet 1040, following step (g), such that the at least one opening 1030 faces the inner GI wall 1052. In further such embodiments, the method further comprises step (i) of continuously altering the direction of the magnetic field induced by the at least one external magnet 1040, following step (h), thereby shaking the capsule 1001, while the at least one opening 1030 remains facing the inner GI wall 1052. Advantageously, the steps disclosed herein can disperse or spread the pressure which capsule 1001 may generate on the inner GI wall 1052 while the at least one opening 1030 is sealed against it, thereby to prevent irritation to the GI tissue.

Although not treated in full detail, it should be readily understood that capsule 1001 and drug delivery system 1000 may combine various configurations and/or aspects of the capsules and drug delivery systems described herein with accordance to the principles of the present invention.

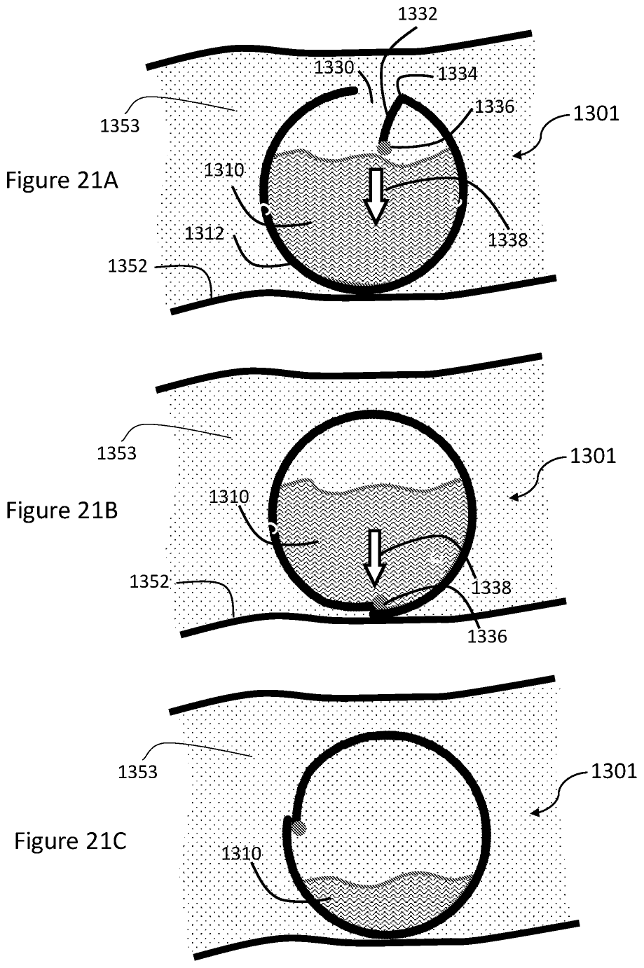

According to yet another aspect, there is provided a capsule 1301. Reference is now made to FIGS. 21A-21C. FIGS. 21A-21C constitutes as cross-sectional views of capsule 1301, according to some embodiments of the present invention.

According to some embodiments, capsule 1301 is configured for oral administration and for magnetically controlled drug release of a pharmaceutical composition 1310 at a desired location in the GI tract, wherein capsule 1301 comprises at least one drug chamber comprising at least one opening 1330 located at the surface of the drug chamber, wherein the drug chamber is adapted to contain therewithin a pharmaceutical composition; and at least one stationary magnet disposed within the drug chamber (not shown). According to some embodiments, capsule 1301 further comprises at least one opening cover 1332, juxtaposed with at least one opening 1330 by at least one hinge 1334, a spring, a flexible or elastic element and the like. A metallic element 1336 configured to be magnetized is attached to opening cover 1332. Hinge 1334 is configured to allow opening cover 1332 to open inward towards the interior of capsule 1301, in the direction of direction arrow 1338. Although a single opening 1330, a single opening cover 1332, a single hinge 1334 and a single metallic element 1336 are described, this is not meant to be limiting in any way. According to some embodiments, a plurality of openings 1330 are provided, each opening 1330 configured to be sealed by a respective opening cover 1332, with a respective hinge 1334 and metallic element 1336.

According to some embodiments, the shape and dimensions of opening cover 1332 are configured to interact with the at least one opening 1330 and thereby seal the at least one opening 1330. According to some embodiments, the opening cover 1332 extends from a first end to a second end, the length between the first end and the second end being equal to, or greater than the diameter of the at least one opening 1330. Although the opening cover 1332 and the hinge 1334 are described as separate elements, this is not meant to be limiting in any way, and the opening cover 1332 can be an extension of the hinge 1334.

According to some embodiments, hinge 1334 comprise a plurality of hinges 1334. According to some embodiments, hinge 1334 is selected from a live hinge, butt hinge, piano hinge, butterfly hinge, flush hinge, spring hinge, gate hinge, or any other element known in the art suitable to connect opening 1330 with opening cover 1332. According to some embodiments, hinge 1334 is a live hinge.

The terms “live hinge” or “living hinge” as used herein are interchangeable, and refer to a hinge which is formed at a joint between two portions of the same piece of material or device, and is optionally made from the same material as the two portions of material it joins. The live hinge can allow hinged movement between the portions of the piece of material on opposite sides of the hinge by flexure or bending of the material along a hinge line.

According to some embodiments, opening cover 1332 is made from the same material in which capsule 1301 is made of.

According to some embodiments, capsule 1301 further comprises an external housing 1312 configured to encompass the capsule 1301.

According to some embodiments, hinge 1334 is a live hinge configured to allow hinged movement between opening cover 1332 and external housing 1312, on opposite sides of hinge 1334.

Opening cover 1332 can be in one of two position: a closed position in which opening cover 1332 covers opening 1330 and prevents pharmaceutical composition 1310 from flowing out of the capsule; and an open position in which opening cover 1332 is bended towards the interior of capsule 1301, in the direction of direction arrow 1338, as illustrated at FIG. 21A, thereby allowing pharmaceutical composition 1310 to be released into the interior of the GI tract 1353 and/or allowing fluids from the GI tract to enter capsule 1301 and mix with pharmaceutical composition 1310 which in turns may flow out of capsule 1301 towards the GI tract.

According to some embodiments, metallic element 1336 is attached to opening cover 1332 at a surface edge of opening cover 1332, as illustrated at FIG. 21A. It is noted that the attachment of metallic element 1336 to opening cover 1332 can be in any location, including on a portion of hinge 1334 that extends from opening cover 1332. According to some embodiments, metallic element 1336 is in a form of an elongated strip (not shown), attached to an inner surface edge of opening cover 1332. It should be understood that metallic element 1336 does not interrupt from opening cover 1332 to maintain a closed position.

According to some embodiments, metallic element 1336 comprises a ferromagnetic compound, such as but not limited to, iron, iron oxide, neodymium iron boron, nickel, aluminum nickel cobalt, cobalt, samarium cobalt, and any combination thereof. Each possibility represents a separate embodiment of the present invention. In further embodiments, metallic element 1336 comprise a ferromagnetic compound comprising soft iron.

According to some embodiments, hinge 1334 is configured to maintain opening 1330 sealed (by opening cover 1332), thereby preventing pharmaceutical composition 1310 from leaking from capsule 1301 in an undesired location. According to some embodiments, hinge 1334 comprise at least one spring (not shown) configured to press opening cover 1332 onto opening 1330, thereby sealing it and preventing leakage of pharmaceutical composition 1310 from the capsule. According to some further embodiments, the force generated by the magnetic field between the metallic element 1336 and the at least one external magnet can be greater than the force applied by the at least one spring. Thus, when capsule 1301 is secured against the inner GI wall 1352 at the desired location of the GI tract, so that at least one opening 1330 is facing the interior of the GI tract 1353, the metallic element 1336 can be configured to be magnetized and to allow opening cover 1332 to open inward towards the interior of capsule 1301.Therefore, the at least one opening cover 1332 is configured to seal the at least one opening 1330 responsive to the at least one metallic element 1336 being magnetized.

According to some embodiments, the cover 1332 is opened and closed responsive to the magnetic field of the at least one external magnet. Particularly, when the capsule 1301 is oriented such that the at least one opening 1330 faces the interior of GI tract 1353, i.e. faces away from the at least one external magnet, the metallic element 1336 is pulled towards the at least one external magnet, thereby opening the cover 1332. Similarly, when the capsule 1301 is oriented such that the at least one opening 1330 faces the inner GI wall 1352, i.e. faces the at least one external magnet, the metallic element 1336 is pulled towards the at least one external magnet, thereby closing the cover 1332 and sealing the opening 1330.

According to some embodiments, the at least one stationary magnet is configured to secure capsule 1301 against the inner GI wall 1352 at the desired location of the GI tract. According to some embodiments, capsule 1301 is configured to controllably release pharmaceutical composition 1310 through at least one opening 1330, into the interior of the GI tract 1353, in response to a change in the direction of an external magnetic field, induced by at least one external magnet. Once capsule 1301 is secured against the inner GI wall 1352 at the desired location of the GI tract, so that at least one opening 1330 is facing the interior of the GI tract 1353, metallic element 1336 is configured to be magnetized and to allow opening cover 1332 to open inward towards the interior of capsule 1301, in the direction of direction arrow 1338, thereby to release pharmaceutical composition 1310 through at least one opening 1330, into the interior of the GI tract 1353 and/or allowing fluids from the GI tract to enter capsule 1301, mix with pharmaceutical composition 1310 which in turns may flow out of capsule 1301 towards the GI tract.

According to some embodiments, metallic element 1336 is configured to be magnetized and is magnetically attracted to the at least one external magnet, thereby allowing opening cover 1332 to open inward towards the interior of capsule 1301, and release pharmaceutical composition 1310 through at least one opening 1330, into the interior of the GI tract 1353.

FIG. 21B illustrates the closed position of opening cover 1332, wherein capsule 1301 is rotated so that the at least one opening 1330 is facing the inner GI wall 1352. According to some embodiments, when capsule 1301 is rotated so that the at least one opening 1330 is facing the inner GI wall 1352, opening cover 1332 is configured to adequately seal the at least one opening 1330 and to prevent leakage of pharmaceutical composition 1310 from capsule 1301. According to further embodiments, metallic element 1336 is configured to be magnetized and is magnetically attracted to the at least one external magnet, thereby allowing opening cover 1332 to move in the direction of direction arrow 1338 and to adequately seal the at least one opening 1330.

Advantageously, the capsule disclosed herein is having an opening cover 1332 comprising metallic element 1336 configured to be magnetized, such that when the capsule is rotated and the at least one opening 1330 is facing inner GI wall 1352, which can be a curvilinear inner GI wall, the at least one opening 1330 is adequately sealed against the opening cover 1332, as illustrated at FIG. 21B. This configuration prevents leakage of the pharmaceutical composition from the capsule and enables controlled release of the pharmaceutical composition.

FIG. 21C illustrates the closed position of opening cover 1332, following the controlled release of an affective amount of pharmaceutical composition 1310, wherein capsule 1301 is about to be removed from the GI tract by changes in the magnetic field induced by the at least one external magnet. In some embodiments, following the controlled release of an affective amount of pharmaceutical composition 1310 over time changes in the magnetic field induced by the at least one external magnet causes capsule 1301 to be eventually released from body, though the GI tract. In further such embodiments, hinge 1334 is configured to place opening cover 1332 over opening 1330, thereby sealing it and thus preventing leakage of any remaining portions of pharmaceutical composition 1310 from the capsule 1301.

According to some embodiments, by weakening the magnitude of the magnetic field (e.g. by removing the at least one external magnet away from the body), the magnetic force residing between metallic element 1336 and the at least one external magnet is smaller than the force required from opening cover 1332 to seal opening 1330, therefore when capsule 1301 is no longer secured against the inner GI wall 1352 following the controlled release of an affective amount of pharmaceutical composition 1310, hinge 1334 is configured to place opening cover 1332 over opening 1330, thereby sealing it and preventing leakage of any remaining portions of pharmaceutical composition 1310 from the capsule 1301.

Although not treated in full detail, it should be readily understood that capsule 1301 may combine various configurations and/or aspects of the capsules described herein with accordance to the principles of the present invention.

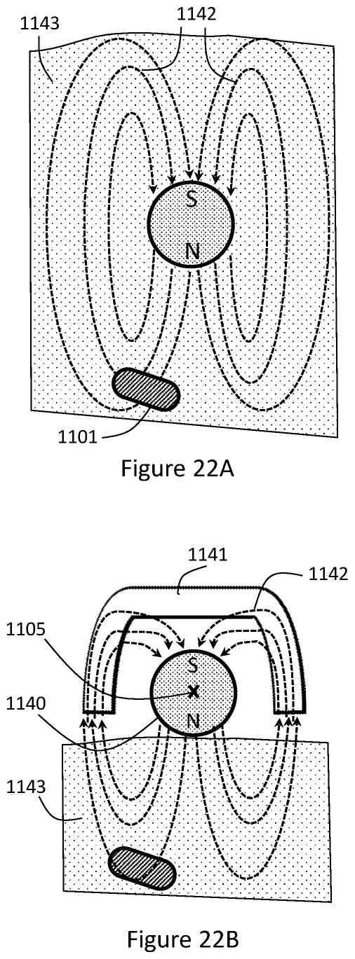

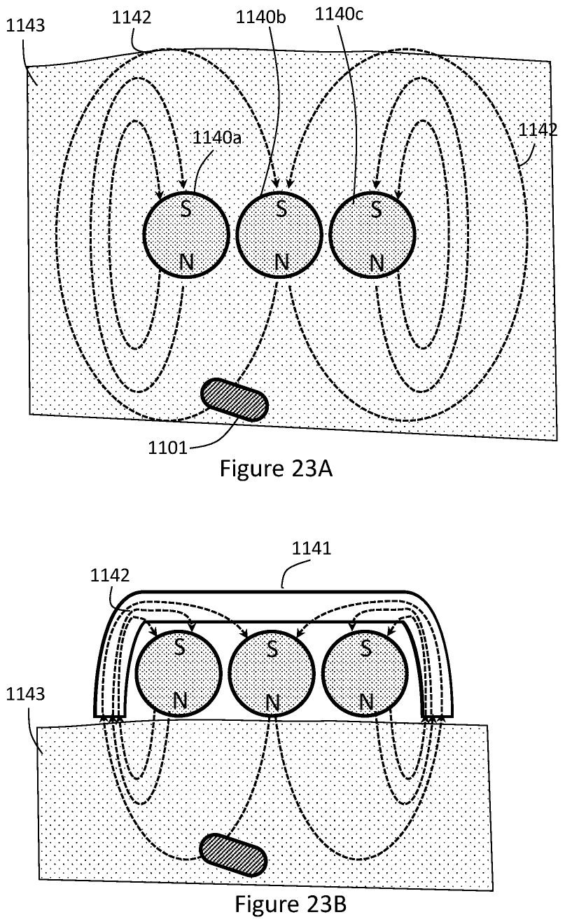

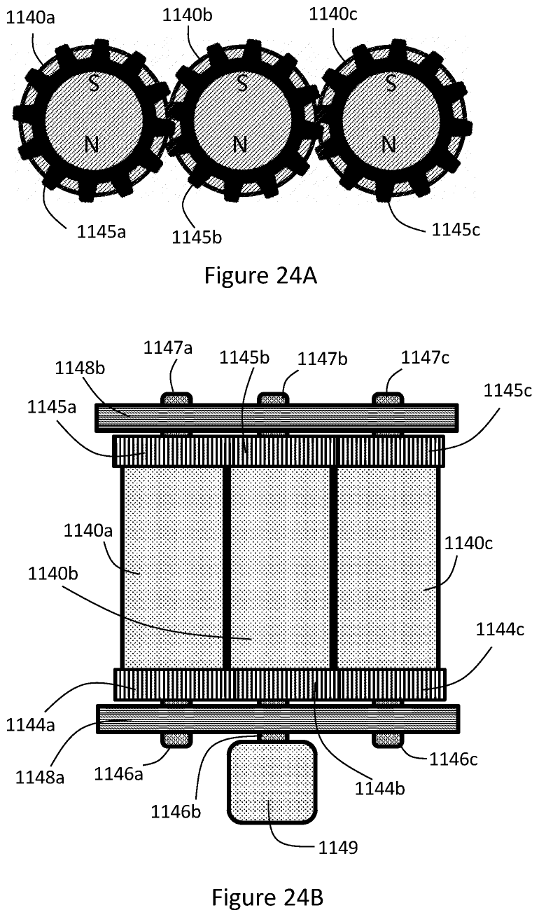

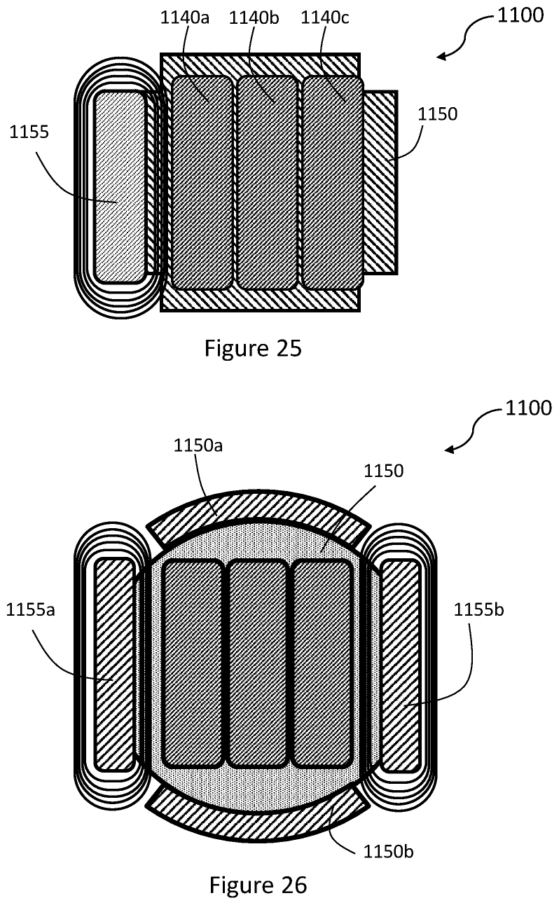

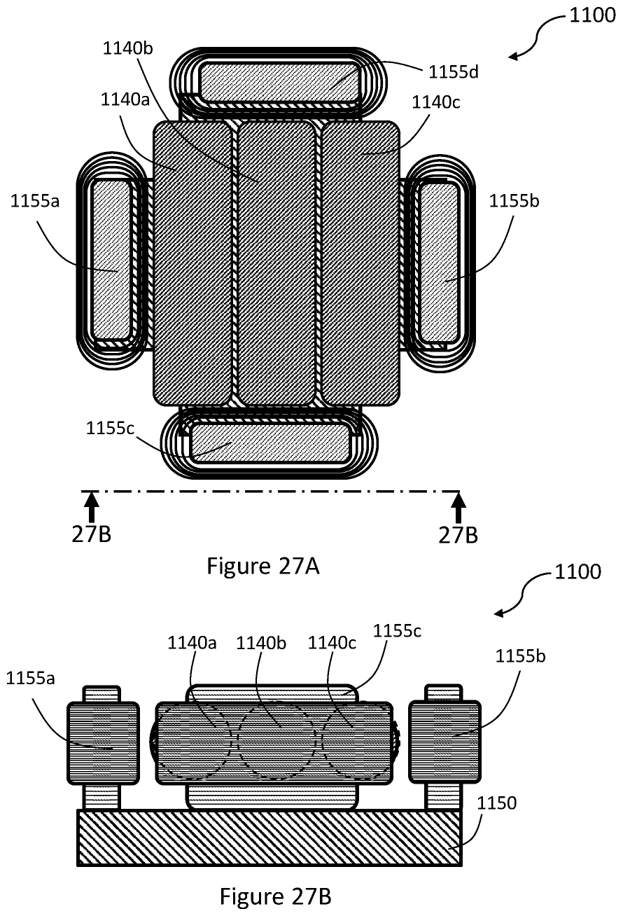

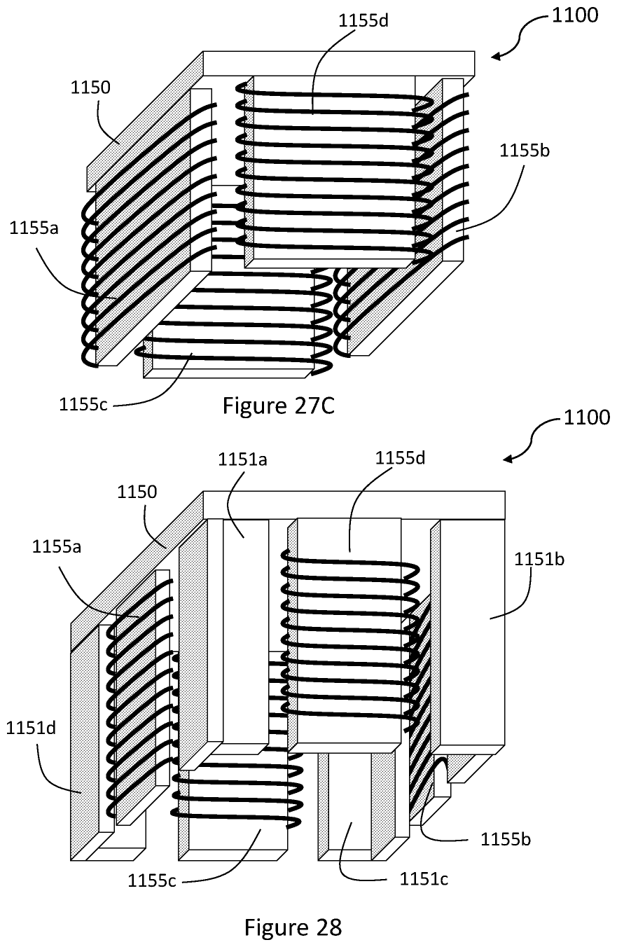

According to yet another aspect, there is provided an external magnet kit 1100. Reference is now made to FIGS. 22A-28. FIG. 22A constitutes a schematic illustration of magnetic flux in the presence of a single magnet. FIG. 22B constitutes a schematic illustration of magnetic flux in the presence of a single magnet in the context of external magnet kit 1100, according to some embodiments of the present invention. FIG. 23A constitutes a schematic illustration of magnetic flux in the presence of three magnets. FIG. 23B constitutes a schematic illustration of magnetic flux in the presence of three magnets in the context of external magnet kit 1100, according to some embodiments of the present invention. FIG. 24A constitutes a side-view of magnets 1140a-c through different embodiments of the present invention. FIG. 24B constitutes a top-view of magnets 1140a-c held by rigid members 1148a-b, through different embodiments of the present invention. FIGS. 25-27A constitutes a top-view of external magnet kit 1100 through different embodiments of the present invention. FIG. 27B constitutes a side-view of external magnet kit 1100 taken on line 27B-27B of FIG. 27A, through different embodiments of the present invention. FIGS. 27C-28 constitutes a view in perspective of external magnet kit 1100, from a front-left angle, through different embodiments of the present invention.

According to some embodiments, the external magnet kit 1100 comprises at least one external magnet 1140 configured to generate an external magnetic field 1143; at least one flux limiting component; and fastening means, wherein the fastening means are configured to attach the external magnet kit 1100 to the skin of a user, and wherein the flux limiting component is configured to attenuate or nullify the intensity of the magnetic field induced by the at least one external magnet 1140, which is directed to the outside environment (i.e., in a direction facing away from the capsule).

According to some embodiments, the at least one flux limiting component is configured to limit the fraction of the external magnetic field 1143 extending outward from the at least one external magnet 1140 towards the outer environment. In further embodiments, the fraction of the external magnetic field 1143 extending inward towards the body of the user is unaffected by the at least one flux limiting component.

According to some embodiments, the at least one flux limiting component comprise a flux limiting cover 1141, configured to form a physical shield around at least one surface of the at least one external magnet facing the outer environment.

Flux lines 1142 of the at least one external magnet 1140 flow from the north pole of the at least one external magnet 1140 to the body of a user, passing through the skin and the GI tract (not shown), on towards the outer environment through the flux limiting cover 1141, and finally to the south pole of the at least one external magnet 1140. In some embodiments, the fraction of the flux lines 1142 passing through the flux limiting cover 1141 extend outwards from the at least one external magnet 1140 towards the outer environment. It should be understood that the schematic depiction of magnetic flux lines in FIGS. 22A-23B are for purposes of explanation only and that varying and/or additional flux lines may be present.

According to some embodiments, the external magnetic field 1143 induced by the at least one external magnet 1140 is configured to control the movement of a capsule 1101 along the GI tract. In further embodiments, said capsule 1101 comprises at least one stationary magnet disposed within and at least one drug chamber, wherein the at least one drug chamber comprises at least one opening located on, or embedded within, the surface of the drug chamber, and at least one pharmaceutical composition comprising at least one pharmaceutical active agent. Although not treated in full detail, it should be readily understood that capsule 1101 may combine various configurations and/or aspects of the capsules described herein above with accordance to the principles of the present invention.

FIG. 22A illustrates the movement of capsule 1101 inside the GI tract (not shown) along flux lines 1142 within the external magnetic field 1143, without the flux limiting cover 1141. It should be noted that without the flux limiting cover 1141 the external magnetic field 1143 extends in all directions, including towards the outer environment.

The present inventors have discovered that when a fraction of the external magnetic field 1143 extends towards the outer environment, it can attract foreign external objects towards the external magnet kit 1100. Such an attraction may endanger the user or harm the external magnet kit 1100. Additionally, the fraction of the external magnetic field 1143 extending towards the outer environment may disturb external objects which interact with and/or are affected by magnetic fields.

Advantageously, in order to overcome this issue and to limit the fraction of the external magnetic field 1143 extending towards the outer environment, the external magnet kit 1100 comprises the flux limiting cover 1141, which is configured to divert part of the magnetic flux lines 1142 to pass therethrough, and therefore to decrease the fraction of magnetic flux lines extending outward from the at least one external magnet 1140 towards the outer environment, as illustrated at FIG. 22B.

According to some embodiments, the flux limiting cover 1141 forms a physical shield around at least one surface of the at least one external magnet facing the outer environment. According to some embodiments, the flux limiting cover 1141 is configured to cover the external surfaces of the at least one external magnet 1140, wherein said surfaces are extending towards the outer environment. In further embodiments, the flux limiting cover 1141 is configured to accommodate the at least one external magnet 1140 therein. In still further embodiments, the flux limiting cover 1141 does not cover the surface of the at least one external magnet 1140 oriented towards the skin of a user.

According to some embodiments, the flux limiting cover 1141 comprises a ferromagnetic compound, such as but not limited to, iron, iron oxide, neodymium iron boron, nickel, aluminum nickel cobalt, cobalt, samarium cobalt, and any combination thereof. Each possibility represents a separate embodiment of the present invention.

According to some embodiments, the at least one external magnet 1140 is configured to rotate around its central longitudinal axis 1105 (going through the center of the magnet 1140, see FIG. 22B). In further embodiments, the rotation of the at least one external magnet 1140 is configured to induce a change in the direction of the external magnetic field 1143.

The rotation of the at least one external magnet 1140 can also weaken/nullify/or stop the external magnetic field 1143, thereby cause capsule 1101 to be removed from the body and the GI tract via peristaltic movement of the GI tract and/or by fluid flow along the GI tract. According to some embodiments, the rotation of the at least one external magnet 1140 is further configured to divert at least a portion of the magnetic flux lines 1142 to pass through flux limiting cover 1141, so that the fraction of magnetic flux lines passing through the body of the user will be diminished or nullified, resulting in the removal of capsule 1101 from the GI tract and the body. According to further embodiments, the rotation of the at least one external magnet 1140 by 90° or 270° is configured to divert at least a portion of the magnetic flux lines 1142 to pass through flux limiting cover 1141, so that the fraction of magnetic flux lines passing through the body of the user will be diminished or nullified. In some embodiments, the external magnet kit 1100 further comprise a control system, configured to control the rotation the at least one external magnet 1140. Said control system could be automatic.

In some embodiments, the external magnet kits of the present invention may comprise a control system (e.g., control system 380) as presented herein, configured to control the operation of at least one of the various components of the system, such as but not limited to, the rotation of the at least one external magnet (such as external magnet 340, 740, 940, 1040, 1140, etc.), the movement of the threaded inner magnet 746, the operation of the at least one electromagnet, and any combination thereof.

Advantageously, capsule 1101 can be optionally removed from the GI tract and the body by the rotation of the at least one external magnet 1140, which can be controlled by said control system, and not solely by the manual removal of the at least one external magnet 1140 by the user. This option is beneficial, particularly for safety measures, in case the user fails to manually remove the at least one external magnet 1140 in due time, the control system can be programmed to cause capsule 1101 to be removed from the GI tract automatically utilizing a predetermined schedule or according to live readings from capsule 1101.

In some embodiments, the at least one external magnet 1140 is cylinder shaped.

Although not treated in full detail, it should be readily understood that the at least one external magnet 1140 may combine various configurations and/or aspects of the external magnets described herein above with accordance to the principles of the present invention.

FIGS. 23A-23B illustrates yet another embodiment of the external magnet kit 1100 which differs from the embodiments of FIGS. 22A-22B in that the at least one external magnet 1140 comprise a plurality of external magnets. According to some embodiments, the at least one external magnet 1140 comprise a first external magnet 1140a, a second external magnet 1140b, and a third external magnet 1140c. In some embodiments, the flux limiting cover 1141 is configured to limit the external magnetic field 1143 extending outward from the first external magnet 1140a, the second external magnet 1140b, and the third external magnet 1140c towards the outer environment.

According to some embodiments, the first external magnet 1140a, the second external magnet 1140b, and the third external magnet 1140c (termed herein “external magnets 1140a-c”) are configured to generate an external magnetic field 1143. Flux lines 1142 originating from the external magnets 1140a-c flow from the north pole of the external magnets 1140a-c to the body of a user, passing through the skin and the GI tract (not shown), on towards the outer environment through the flux limiting cover 1141, and finally to the south pole of the external magnets 1140a-c. In some embodiments, the fraction of the flux lines 1142 passing through the flux limiting cover 1141 are extending outward from the external magnets 1140a-c towards the outer environment.

FIG. 23A illustrate the movement of capsule 1101 inside the GI tract (not shown) along flux lines 1142 within the external magnetic field 1143, without the flux limiting cover 1141. It should be noted that without the flux limiting cover 1141 the external magnetic field 1143 extends in all directions, including towards the outer environment. According to some embodiments, the flux limiting cover 1141 is configured to divert part of the magnetic flux lines 1142 to pass through said flux limiting cover 1141, and therefore to decrease the fraction of magnetic flux lines extending outward from the external magnets 1140a-c towards the outer environment, as illustrated at FIG. 23B.

According to some embodiments, the flux limiting cover 1141 is configured to cover the external surfaces of the external magnets 1140a-c, wherein said surfaces are extending towards the outer environment. In further embodiments, the flux limiting cover 1141 does not cover the surface of the external magnets 1140a-c oriented towards the skin of a user.

It is noted that the same functional and structural principles disclosed for the at least one external magnet 1140 similarly apply to the first external magnet 1140a, the second external magnet 1140b, and the third external magnet 1140c.

According to some embodiments, the external magnets 1140a-c are configured to rotate around their respective central longitudinal axes. In further embodiments, the rotation of the external magnets 1140a-c is configured to induce a change in the direction of the external magnetic field 1143.

FIGS. 24A-24B illustrates yet another embodiment of the external magnet kit 1100 which differs from the embodiments of FIGS. 23A-23B in that the external magnet kit 1100 further comprises a rotating member 1149, the rotating member 1149 comprising a plurality of gears and an actuator 1149, configured to rotate each of the first external magnet 1140a, the second external magnet 1140b, and the third external magnet 1140c around their respective central axes.

While FIGS. 24A-24B depict embodiments of a specific external magnet kit 1100 comprising a plurality of gears, it will be understood by those skilled in the art that other transmission systems can be implemented, including any set of interworking gears, cogwheels, racks, bearings, screws, chains, shafts, pulleys and conveyor belts, configured to transform rotational motion of actuator 1149 to rotational motion of the first external magnet 1140a, the second external magnet 1140b, and the third external magnet 1140c in the desired directions.

According to some embodiments, the first external magnet 1140a is connected to a proximal first gear 1144a at one end and to a distal first gear 1145a at the other end. In further embodiments, the second external magnet 1140b is connected to a proximal second gear 1144b at one end and to a distal second gear 1145b at the other end. In still further embodiments, the third external magnet 1140c is connected to a proximal third gear 1144c at one end and to a distal third gear 1145c at the other end.

Within the context of this application, the term “proximal” generally refers to the side or end of any device or a component of a device, which is closer to the actuator 1149.

Within the context of this application, the term “distal” generally refers to the side or end of any device or a component of a device, which is opposite the “proximal” side, and is farther from actuator 1149.

According to some embodiments, the first external magnet 1140a is connected to a proximal first shaft 1146a at one end, and to a distal first shaft 1147a at the other end. In further embodiments, the second external magnet 1140b is connected to a proximal second shaft 1146b at one end, and to a distal second shaft 1147b at the other end. In still further embodiments, the third external magnet 1140c is connected to a proximal third shaft 1146c at one end, and to a distal third shaft 1147c at the other end.

In some embodiments, the proximal first shaft 1146a, proximal second shaft 1146b, and the proximal third shaft 1146c are supported by a first rigid member 1148a so that the proximal first shaft 1146a, proximal second shaft 1146b, and the proximal third shaft 1146c are able to rotate around their respective central longitudinal axis. In some embodiments, the distal first shaft 1147a, distal second shaft 1147b, and distal third shaft 1147c are supported by a second rigid member 1148b so that the distal first shaft 1147a, distal second shaft 1147b, and distal third shaft 1147c are able to rotate around their respective central longitudinal axis.

According to some embodiments, the proximal second shaft 1146b is connected to an actuator 1149, configured to rotate the second external magnet 1140b around its central longitudinal axis, resulting in the rotation of the proximal second gear 1144b and the distal second gear 1145b around themselves, respectively.

The term “actuator”, as used herein, refers to any powered actuator known in the art for providing rotational motion, such as an electric motor, a solenoid, and the like.

The proximal first gear 1144a, proximal second gear 1144b, and the proximal third gear 1144c are interconnected with each other, such that when the proximal second gear 1144b rotates around itself, the proximal first gear 1144a and the proximal third gear 1144c are each rotated in a direction opposing the rotational direction of the proximal second gear 1144b. The distal first gear 1145a, distal second gear 1145b, and distal third gear 1145c are interconnected with each other, such that when the distal second gear 1145b rotates around itself, the distal first gear 1145a and the distal third gear 1145c are rotated in a direction opposing the rotational direction of the distal second gear 1144b.

According to some embodiments, the rotation of the second external magnet 1140b around its central longitudinal axis is configured to rotate the first external magnet 1140a and the third external magnet 1140c in the same direction. In further embodiments, the rotation of the first external magnet 1140a, the second external magnet 1140b, and the third external magnet 1140c is configured to induce a change in the direction of the external magnetic field 1143.

In some embodiments, the external magnet kit 1100 comprises a control system, configured to control the rotation the first external magnet 1140a, the second external magnet 1140b, and the third external magnet 1140c, around their respective central longitudinal axis, wherein said rotation is configured to change the direction of the external magnetic field 1143. Said control system could be automatic.

FIG. 25 illustrates yet another embodiment of the external magnet kit 1100 which differs from the embodiments of FIGS. 24A-24B in that the external magnet kit 1100 further comprises a first base 1150; and wherein the flux limiting component comprises at least one electromagnet 1155, wherein the first base 1150 is attached to the fastening means and is further attached, directly or indirectly, to the first external magnet 1140a, the second external magnet 1140b, the third external magnet 1140c, and the at least one electromagnet 1155, thereby providing stable support to thereto. According to some embodiments, external magnet kit 1100 presented at FIG. 25 further comprises the plurality of gears and the actuator as presented at FIGS. 24A-24B (not shown). According to some embodiments, the first base 1150 faces the body of the user and is secured thereto by the fastening means. Particularly, FIG. 25 illustrates a top view of the external magnet kit 1100, where the bottom of the external magnet kit 1100 faces the patient.

According to some embodiments, the first base 1150 is configured to support the first rigid member 1148a and the second rigid member 1148b.

According to some embodiments, the at least one electromagnet 1155 is configured to limit the fraction of the external magnetic field 1143 extending outward towards the outer environment, induced by the first external magnet 1140a, the second external magnet 1140b, and the third external magnet 1140c. In further embodiments, the fraction of the external magnetic field 1143 extending inward towards the body of the user is unaffected by the at least one electromagnet 1155.

According to some embodiments, the at least one electromagnet 1155 is configured to generate a magnetic field, wherein said magnetic field is adapted to decrease or to nullify the fraction of the external magnetic field 1143 generated by the first external magnet 1140a, the second external magnet 1140b, and the third external magnet 1140c, extending outward from the first external magnet 1140a, the second external magnet 1140b, and the third external magnet 1140c towards the outer environment.

According to some embodiments, the at least one electromagnet 1155 is an electropermanent magnet.

According to some embodiments, external magnet kit 1100 further comprises a second base (not shown), connected to the first base 1150 by a plurality of surface plates (not shown). In further embodiments, the first external magnet 1140a, the second external magnet 1140b, and the third external magnet 1140c are disposed therebetween the first base 1150 and the second base surface. In still further embodiments, the first base 1150, the second base surface, and the plurality of surface plates are configured to accommodate the first external magnet 1140a, the second external magnet 1140b, and the third external magnet 1140c therein.

According to some embodiments, the first base 1150 and the second base are square shaped. However, it is to be understood that the first base 1150 and the second base fulfill the same function when otherwise shaped, as a circle, rectangle, triangle, rhombus, parallelogram, oval, star, pentagon, hexagon, octagon or any other polygon.

According to some embodiments, the rotation of the first external magnet 1140a, the second external magnet 1140b, and the third external magnet 1140c is configured to cause the at least one electromagnet 1155 to limit or to nullify the fraction of the external magnetic field 1143 passing through the body of the user, resulting in the removal of capsule 1101 from the GI tract and the body of the user. According to further embodiments, the rotation of the first external magnet 1140a, the second external magnet 1140b, and the third external magnet 1140c by 90° or 270° around their respective central axes is configured to divert at least a portion of the magnetic flux lines passing through the body of the user, so that the fraction of magnetic flux lines passing through the body of the user will be diminished or nullified. In some embodiments, the external magnet kit 1100 further comprises a control system, configured to control the rotation the at least one external magnet 1140. Said control system could be automatic.

Advantageously, capsule 1101 can be optionally removed from the GI tract and the body of the user by the rotation of the first external magnet 1140a, the second external magnet 1140b, and the third external magnet 1140c, which can be controlled by said control system, and not solely by the manual removal of said external magnets 1140a-c by the user. This option is beneficial, particularly for safety measures, in case the user fails to manually remove external magnets 1140a-c in due time, the control system can be programmed to cause capsule 1101 to be removed from the GI tract automatically utilizing a predetermined schedule or according to live readings from capsule 1101.

FIG. 26 illustrates yet another embodiment of the external magnet kit 1100 which differs from the embodiments of FIG. 25 in that the first base 1150 is circle shaped, and the at least one electromagnet 1155 comprise a first electromagnet 1155a and a second electromagnet 1155b, supported by the first base 1150. According to some embodiments, external magnet kit 1100 presented at FIG. 26 further comprise the plurality of gears and the actuator as presented at FIGS. 24A-24B (not shown).

According to some embodiments, first electromagnet 1155a and the second electromagnet 1155b are configured to generate separate magnetic fields, wherein said magnetic fields are adapted to decrease or to nullify the fraction of the external magnetic field 1143 generated by the first external magnet 1140a, the second external magnet 1140b, and the third external magnet 1140c, extending outward from the first external magnet 1140a, the second external magnet 1140b, and the third external magnet 1140c towards the outer environment.

According to some embodiments, the second base is circle shaped, and is connected to the first base 1150 by a first circular surface plate 1150a and by a second circular surface plate 1150b.

FIGS. 27A-27C illustrates yet another embodiment of the external magnet kit 1100 which differs from the embodiments of FIG. 25 in that the at least one electromagnet 1155 comprise a first electromagnet 1155a, a second electromagnet 1155b, a third electromagnet 1155c, and a fourth electromagnet 1155d, and wherein the first base 1150 is configured to support the first electromagnet 1155a, second electromagnet 1155b, third electromagnet 1155c, and the fourth electromagnet 1155d. According to some embodiments, external magnet kit 1100 presented at FIGS. 27A-27C further comprises the plurality of gears and the actuator as presented at FIGS. 24A-24B (not shown). According to some embodiments, the first base 1150 is facing the body of the user and is secured thereto by the fastening means. Particularly, FIG. 27A illustrates a top view of the external magnet kit 1100, such that the bottom of the external magnet kit 1100 faces the body of the user. Similarly, FIG. 27B illustrates a cut-away side view of the external magnet kit 1100, such that the bottom of the external magnet kit 1100 faces the body of the user. FIG. 27C illustrates a perspective view of the external magnet kit 1100, such that the top of the external magnet kit 1100 faces the body of the user.

According to some embodiments, the first electromagnet 1155a, second electromagnet 1155b, third electromagnet 1155c, and the fourth electromagnet 1155d are configured to generate separate magnetic fields, wherein said magnetic fields are adapted to decrease or to nullify the fraction of the external magnetic field 1143 generated by the first external magnet 1140a, the second external magnet 1140b, and the third external magnet 1140c, extending outward towards the outer environment.

FIG. 27B constitutes a side-view of external magnet kit 1100 taken on line 27B-27B of FIG. 27A, through different embodiments of the present invention. The third electromagnet 1155c hides the fourth electromagnet 1155d (not shown) and the first external magnet 1140a, the second external magnet 1140b, and the third external magnet 1140c (illustrated by dotted circles).

FIG. 27C constitutes a view in perspective of external magnet kit 1100, from a front-left angle, through different embodiments of the present invention. The first external magnet 1140a, the second external magnet 1140b, and the third external magnet 1140c (not shown) are disposed therebetween the first electromagnet 1155a, second electromagnet 1155b, third electromagnet 1155c, and the fourth electromagnet 1155d, and are supported by the first base 1150.

FIG. 28 illustrates yet another embodiment of the external magnet kit 1100 which differs from the embodiments of FIG. 27C in that the external magnet kit 1100 further comprise a first corner surface plate 1151a, a second corner surface plate 1151b, a third corner surface plate 1151c, and a fourth corner surface plate 1151d. The first corner surface plate 1151a is spaced from the second corner surface plate 1151b by the fourth electromagnet 1155d. The second corner surface plate 1151b is spaced from the third corner surface plate 1151c by the second electromagnet 1155b. The third corner surface plate 1151c is spaced from the fourth corner surface plate 1151d by the third electromagnet 1155c. The fourth corner surface plate 1151d is spaced from the first corner surface plate 1151a by the first electromagnet 1155a. According to some embodiments, external magnet kit 1100 presented at FIG. 28 further comprises the plurality of gears and the actuator as presented at FIGS. 24A-24B (not shown).

The first electromagnet 1155a is supported by the first base 1150 and is disposed between the first corner surface plate 1151a and the fourth corner surface plate 1151d. The second electromagnet 1155b is supported by the first base 1150 and is disposed between the second corner surface plate 1151b and the third corner surface plate 1151c. The third electromagnet 1155c is supported by the first base 1150 and is disposed between the third corner surface plate 1151c and the fourth corner surface plate 1151d. The fourth electromagnet 1155d is supported by the first base 1150 and is disposed between the second corner surface plate 1151b and the first corner surface plate 1151a.

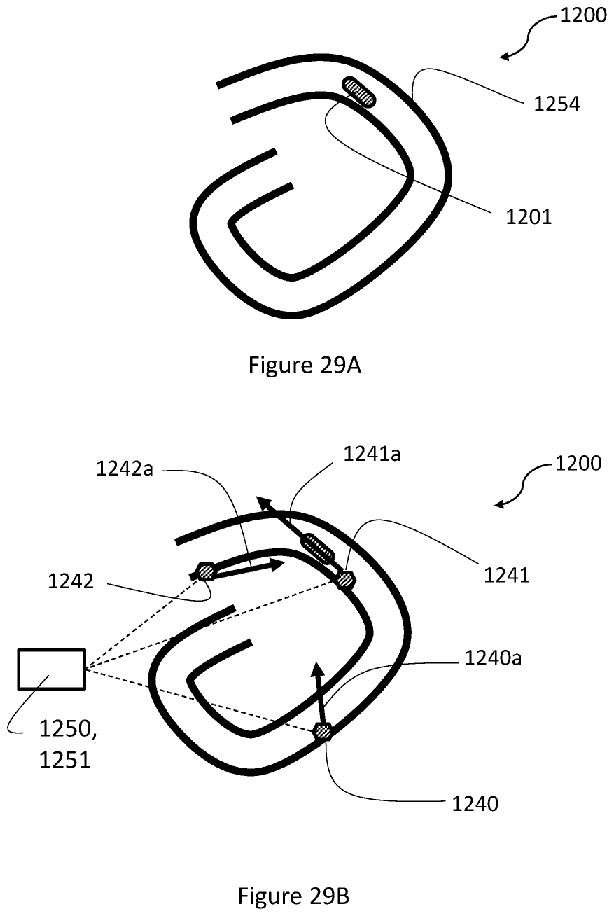

According to yet another aspect, there is provided a system configured to locate the desired location of the GI tract in which the pharmaceutical composition is to be released. Reference is now made to FIGS. 29A-29B. FIGS. 29A-29B constitutes a schematic cross-sectional view of GI tract region 1200, according to some embodiments. According to some embodiments, the system comprises a device configured to measure the position and orientation of a permanent magnet within a three-dimensional region of the GI tract, the device comprising:
fastening means configured to attach the device to the body of a user;
at least three sensors, each configured to detect the presence of the magnetic fields and the direction thereto; and
a controller configured to receive magnetic field data from the at least three sensors and to transmit said data to a processing unit,
   wherein the at least three sensors and the controller are connected to the fastening means, and wherein the processing unit is configured to determine the position and the orientation of the permanent magnet along a three-dimensional region of the GI tract.

According to some embodiments, the magnetic field data comprise the direction, amplitude, or relative change of the magnetic field induced by the permanent magnet within the three-dimensional segment of the GI tract. As used herein, the term “magnetic field vector” refers to the direction and amplitude of the permanent magnet within a three-dimensional segment of the GI tract.

In some embodiments, the processing unit is configured to determine the position and the orientation of the permanent magnet along a plurality of three-dimensional segments along the GI tract. In further embodiments, the processing unit is configured to ensemble magnetic field vectors at corresponding three-dimensional segments along the GI tract, utilizing an algorithm. In still further embodiments, the processing unit is configured to track the route of the permanent magnet through the corresponding three-dimensional segments along the GI tract. In yet still further embodiments, the processing unit maps the corresponding three-dimensional segments and forms a three-dimensional model of the relevant region of the GI tract. In some embodiments, the processing unit is a part of the controller. Alternately, the processing unit may be a separate component than the controller.

According to some embodiments, the processing unit is configured to indicate the desired location along the GI tract in which at least one pharmaceutical active agent is to be released and compare the desired location to the identified location of the permanent magnet to determine whether the capsule has arrived at the desired location.

According to some embodiments, each of the at least three sensors comprises a magnetometer.

In some embodiments, the processing unit is connected to the device. In other embodiments, the controller is configured to receive magnetic field data from the at least three sensors and to transmit said data to an external processing unit. In further embodiments, the controller is configured to wirelessly transmit said data to the external processing unit.

According to some embodiments, the device further comprises a control system. In further embodiments, said control system is configured to receive magnetic field data from the at least three sensors, and to track the route of the permanent magnet through corresponding three-dimensional segments along the GI tract. In yet further embodiments, the control system is configured to compare the location of the tracked permanent magnet to the desired location along the GI tract in which the pharmaceutical composition is to be released. According to some embodiments, the control system is further configured to output the outcome of the comparison, or an indication thereof.

According to some embodiments, there is provided a method for measuring the position and orientation of a permanent magnet within a three-dimensional region of the GI tract, comprising the steps of:
a) providing the device configured to measure the position and orientation of a permanent magnet within a three-dimensional region of the GI tract, as was described herein above;
b) administering, per os, capsule 1201, wherein said capsule 1201 is configured for oral administration and comprise at least one permanent magnet disposed within configured to output a magnetic field external to the capsule 1201; and
c) determining the position and the orientation of the capsule 1201 comprising the permanent magnet along a plurality of corresponding three-dimensional segments along the GI tract.

According to some embodiments, capsule 1201 is configured for oral administration and to travel along the GI tract 1254 by peristaltic movement of the GI tract and/or by fluid flow along the interior of the GI tract, as illustrated at FIG. 29A.

According to some embodiments, the at least three sensors of the device at step (a) comprise a first sensor 1240, a second sensor 1241, and a third sensor 1242, wherein each sensor is located at a different location, as illustrated at FIG. 29B, and wherein the sensors are mounted to the fastening means. The first sensor 1240 is configured to detect magnetic field vector 1240a. The second sensor 1241 is configured to detect magnetic field vector 1241a. The third sensor 1242 is configured to detect magnetic field vector 1242a.

According to some embodiments, the method further comprises assembling magnetic field vectors at corresponding three-dimensional segments along the GI tract, utilizing an algorithm. In further embodiments, the method further comprises tracking the route of the capsule 1201 through the corresponding three-dimensional segments along the GI tract. In still further embodiments, the method further comprises mapping the corresponding three-dimensional segments and forming a three-dimensional model of the relevant region of the GI tract. In yet still further embodiments, the method further comprises comparing the identified location of the permanent magnet (i.e. the location of the capsule) to the desired location along the GI tract in which the at least one pharmaceutical active agent is to be released.

According to some embodiments, a controller 1250 is in operative communication the each one of the sensors as presented herein above, and is configured to receive magnetic field data therefrom and to transmit said data to a processing unit 1251. Alternately, the controller 1250 comprises the processing unit 1251 as an integral part thereof. The processing unit 1251 is configured to execute various calculations as presented herein (e.g., mapping, tracking, detecting, locating, etc.). The controller 1250, the processing unit 1251, or both, can be an integral part of the control system 380 as presented herein above. According to some embodiments, the controller 1250 is identical to the control system 380 as presented herein above.

Alternately, according to some embodiments, the device configured to locate the capsule 1201 comprises at least three sonar sensors configured to detect acoustic signals, wherein the processing unit is configured to determine the position and the orientation of an acoustic element having a predefined resonance frequency. According to some embodiments, the capsule 1201 is configured for oral administration and comprise at least one acoustic element having a predefined resonance frequency. According to some embodiments, said at least three sonar sensors are configured to send audio pulses configured to cause the acoustic element to vibrate at a predefined resonance frequency and generate acoustic signals. In further embodiments, the at least three sonar sensors are further configured to detect the acoustic signals generated by the acoustic element.

Moreover, according to some embodiments, the device configured to locate the capsule 1201 comprises at least two electromagnetic radiation sensors configured to detect reflected electromagnetic radiation, wherein the processing unit is configured to determine the position and the orientation of a reflector element. According to some embodiments, the capsule 1201 is configured for oral administration, is optionally at least partially transparent, and comprises at least one reflector element. According to some embodiments, said at least two electromagnetic radiation sensors are configured to irradiate the body of the user with electromagnetic radiation. In further embodiments, the at least one reflector element is configured to reflect said electromagnetic radiation. In such further embodiments, the at least two electromagnetic radiation sensors are further configured to detect the reflected electromagnetic radiation from the corner reflector element within capsule 1201. In some embodiments, the electromagnetic radiation comprises wavelengths in the range of about 1 mm to about 25 μm (microwave radiation).

Although not treated in full detail, it should be readily understood that various configurations of the capsules and/or drug delivery systems of the present invention may combine at least two different aspects and/or configurations, according to any of the configurations described herein above, with accordance to the principles of the present invention.

Particularly, capsules 301, 401, 501, 601, 701, 801, 901, 1001, 1101, 1201 and 1301 can each exhibit the properties and elements described in relation to other capsules. According to some embodiments, each capsule can comprise: a stationary magnet, such as described in relation to magnets 320, 420, 520, 620, 720 and 920; and/or a non-stationary magnet, such as described in relation to one of magnets 542 and 544. According to some embodiments, each capsule can comprise a mobile member configured to travel within at least one drug chamber, as described in relation to mobile member 373. According to some embodiments, each capsule can comprise a casing, such as described in relation to casing 468. According to some embodiments, each capsule can comprise at least one magnetic shunt, as described in relation to magnetic shunts 575 and 576, and at least one buffer which is configured to erode due to interaction with GI tract fluid, as described in relation to buffer 513. According to some embodiments, each capsule can comprise at least one membrane configured to cover at least one surface of a magnet, as described in relation to membranes 668 and 669 (i.e., buffer 670). According to some embodiments, each capsule can exhibit various shapes, as described in relation to capsule 801. According to some embodiments, each capsule can comprise a circumferential external barrier, as described in relation to circumferential external barrier 835. According to some embodiments, each capsule can comprise at least one opening cover, as described in relation to at least one opening cover 1332.

Similarly, drug delivery systems 300, 700, 900 and 1000 can each exhibit the properties and elements described in relation to other drug delivery systems. According to some embodiments, each drug delivery system can comprise at least one external magnet, as described in relation to external magnets 340, 740, 940, 1040 and 1140. According to some embodiments, each drug delivery system can comprise at least one intermediate magnetic housing comprising at least one inner magnet, as described in relation to intermediate magnetic housing 745. According to some embodiments, each drug delivery system can comprise a rail configured to support movement of at least one external magnet, as described in relation to rail 941. According to some embodiments, each drug delivery system can comprise a flux limiting cover configured to form a physical shield around at least one surface of at least one external magnet facing the outer environment, as described in relation to flux limiting cover 1141. According to some embodiments, each drug delivery system can comprise a plurality of external magnets, as described in relation to external magnet kit 1100. According to some embodiments, each drug delivery system can comprise a suitable mechanism for rotating at least one external magnet, as described in relation to external magnet kit 1100. According to some embodiments, each drug delivery system can comprise one or more electromagnets, such as described in relation to electromagnets 1155. According to some embodiments, each drug delivery system can comprise a plurality of sensors, as described in relation to sensors 1240, 1241 and 1242.

The term “plurality”, as used herein, means more than one.

The term “about”, as used herein, when referring to a measurable value such as an amount, a temporal duration, and the like, is meant to encompass variations of +/−10%, more preferably +/−5%, even more preferably +/−1%, and still more preferably +/−0.1% from the specified value, as such variations are appropriate to the disclosed devices, systems and/or methods.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. No feature described in the context of an embodiment is to be considered an essential feature of that embodiment, unless explicitly specified as such.

Although the invention is described in conjunction with specific embodiments thereof, it is evident that numerous alternatives, modifications and variations that are apparent to those skilled in the art may exist. It is to be understood that the invention is not necessarily limited in its application to the details of construction and the arrangement of the components and/or methods set forth herein. Other embodiments may be practiced, and an embodiment may be carried out in various ways. Accordingly, the invention embraces all such alternatives, modifications and variations that fall within the scope of the appended claims.

1. A capsule configured for oral administration and drug release at a desired location in the gastrointestinal (GI) tract, comprising:
at least one magnet configured to output a magnetic field external to the capsule;
at least one buffer; and
at least one drug chamber comprising at least one opening located at a surface of the at least one drug chamber and at least one pharmaceutical composition,
wherein responsive to an erosion of the at least one buffer, an intensity of the external output magnetic field is reduced.

2. The capsule according to claim 1, wherein the at least one buffer comprises the at least one pharmaceutical composition, and
wherein responsive to an erosion of the at least one pharmaceutical composition, the at least one pharmaceutical composition is gradually released through the at least one opening towards the desired location within the interior of the GI tract.

3. The capsule according to claim 2, further comprising at least one magnetic shunt,
whereby prior to the erosion of the pharmaceutical composition, the pharmaceutical composition is positioned between the at least one magnet and the at least one magnetic shunt such that the at least one magnet does not contact the at least one magnetic shunt.

4. The capsule according to claim 3, wherein responsive to the erosion of the pharmaceutical composition, relative movement is initiated between the at least one magnetic shunt and the at least one magnet such that the at least one magnet approaches the at least one magnetic shunt.

5. The capsule according to claim 2, wherein the at least one magnet comprises a pair of horse-shoe shaped magnets, the at least one pharmaceutical composition separating the pair of horse-shoe shaped magnets.

6. The capsule according to claim 1, wherein the at least one buffer comprises at least one biodegradable membrane.

7. The capsule according to claim 6, wherein the at least one biodegradable membrane separates the at least one magnet from an environment surrounding the capsule.

8. The capsule according to claim 7, wherein responsive to an erosion of the at least one biodegradable membrane, the at least one magnet is exposed to the environment surrounding the capsule.

9. The capsule according to claim 6, wherein the at least one magnet is configured to erode responsive to contact with fluid of the GI tract.

10. The capsule according to claim 6, further comprising a corrosive powder, the at least one biodegradable membrane separating the corrosive powder from an environment surrounding the capsule.

11. The capsule according to claim 10, wherein responsive to an erosion of the at least one biodegradable membrane, the corrosive powder is exposed to the environment surrounding the capsule.

12. The capsule according to claim 10, wherein the at least one magnet is configured to erode responsive to contact with a solution of the corrosive powder and fluid of the GI tract.

13. The capsule according to claim 1, further comprising:
at least one partitioning element positioned so as to partition the drug chamber into two fluidly connected first and second compartments; and
at least one mobile member configured to be magnetized, the at least one mobile member residing within the second compartment.

14. The capsule according to claim 13, wherein the at least one opening comprises a first opening facing the first compartment and a second opening facing the second compartment, and
wherein, responsive to changes in direction of an externally applied magnetic field, the at least one mobile member is configured to alternately move towards and away from the second opening.

15. The capsule according to claim 14, wherein a predetermined volume of the pharmaceutical composition is released through the first opening responsive to the mobile member moving towards the second opening.

16. The capsule according to claim 13, further comprising at least one magnetic shield adapted to magnetically shield the at least one mobile member from the at least one magnet.

17. The capsule according to claim 13, wherein the at least one partitioning element comprises a first partitioning element and a second partitioning element, the second compartment is formed between the first partitioning element and the second partitioning element, and a third compartment is formed between the second partitioning element and an edge of the capsule,
wherein the third compartment is in fluid connection with the second compartment,
wherein the at least one opening comprises a first opening facing the first compartment and a second opening facing the third compartment, and
wherein, responsive to changes in direction of an externally applied magnetic field, the at least one mobile member is configured to alternately move through the second compartment towards the fluid connection to the third compartment and away from the fluid connection to the third compartment.

18. The capsule according to claim 1, further comprising:
a casing encompassing at least the drug chamber; and
at least one spacing element positioned between the drug chamber and the casing,
wherein a predetermined space is defined between the casing and the drug chamber, the predetermined space defined by the at least one spacing element,
wherein the casing is configured to be compressed against the drug chamber, thereby sealing the at least one opening.

19-25. (canceled)

26. A drug delivery system comprising:
the capsule according to claim 1
an external magnet kit comprising at least one external magnet configured to be juxtaposed with the skin of a user at the vicinity of the desired location of the GI tract;
at least one intermediate housing; and
at least one inner magnet, disposed within the at least one intermediate housing,
wherein the at least one intermediate housing is configured to be implanted below the skin of the user, between the at least one external magnet and the desired location of the GI tract,
wherein a flow of the pharmaceutical composition through the at least one opening is controlled responsive to a direction of the magnetic field of the at least one external magnet.

27-29. (canceled)

30. The system according to claim 26, wherein the at least one intermediate housing comprises:
a magnetic shield member; and
a translation member, the translation member configured to translate the at least one inner magnet between a first position and a second position in relation to the magnetic shield member,
wherein, responsive to the magnetic shield member, the intensity of a magnetic field of the at least one inner magnet is greater in the first position than in the second position.

31-41. (canceled)
